(19) United States
(12) Patent Application Publication
(10) Pub. No.: US 2026/0139222 A1
(43) Pub. Date: May 21, 2026

(54) SYSTEMS WITH BIO-ENGINEERED ADHESIVE SILOXANE SUBSTRATE OF TUNABLE STIFFNESS AND METHODS OF USE THEREOF

(71) Applicant: NUTECH VENTURES, Lincoln, NE (US)

(72) Inventor: Srivatsan KIDAMBI, Lincoln, NE (US)

(21) Appl. No.: 19/118,523

(22) PCT Filed: Oct. 6, 2023
(86) PCT No.: PCT/US2023/076292

§ 371 Date: Apr. 4, 2025

Related U.S. Application Data
(60) Provisional application No. 63/378,672, filed on Oct. 6, 2022.

(51) Int. Cl. C12N 5/079; C12M 1/12; C12N 5/071; C12N 5/073

(52) U.S. Cl. CPC C12N 5/0622; C12M 25/04; C12N 5/0603; C12N 5/0671; C12N 2513/00; C12N 2533/30

(57) ABSTRACT

Provided here are cell culture system with tunable stiffness comprising a biocompatible polydimethyl siloxane substrate and a polyelectrolyte multilayer. Also provided here are methods of making and using these systems as research and/or product development models for different tissue and organ systems.

### TECHNICAL FIELD

The disclosure relates to two- or three dimensional cell culture systems with tunable stiffness containing a biocompatible polydimethyl siloxane substrate and a polyelectrolyte multilayer.

### BACKGROUND

The development of animal models allowed for significant progress to be made in biomedical research and drug development. Animal models can be used to study the pathogenesis of disease at different stages; however, they do not capture the changes in disease states including spectrum of the metabolic, inflammatory, and fibrotic responses found in human patients and their use in high-throughput assays are limited. Primary cell models are required for clinical therapy, as well as studies in pharmacology and toxicology. A key challenge in the use of primary cells is in conventional cell culture practice, where the cells tend to lose their extracellular signaling cues and responses. The majority of stiffness studies use collagen gels and matrigel as the matrix. The animal-based source and lack of information on the composition is a major limitation with the use of these gels as they might cause non-physiological responses from the cells. Polyacrylamide gels, commonly used synthetic biomaterials for stiffness studies, are limited because covalent crosslinking of proteins using harsh chemicals is necessary for cell adhesion and the elastic creasing instability of the softer polyacrylamide gel results in non-specific cell behavior.

Biomimetic in vitro models are urgently needed to allow both investigation of the mechanisms of diseases and higher-throughput screening of drugs and drug combinations. In vitro models, such as sandwich cell culture and tissue spheroids, may recapitulate several of the features of diseases, however, they are missing the dynamics of the tissue microenvironment and cell-cell interactions and proper functional reproduction. Organoids derived using pluripotent stem cell (PSC)-derived cells have enormous potential as a replacement for primary cells in drug screening, toxicology and cell replacement therapy, but their genome-wide expression patterns differ strongly from primary cells. Furthermore, current in vitro models have been unable to recapitulate systemic effects induced by external agents/environmental factors in humans, such as chronic liver diseases, fibrosis, aging, preeclampsia, cardiac fibrosis to name a few.

### SUMMARY

Provided here are systems and methods to address these shortcomings of the art and provide other additional or alternative advantages. The disclosure herein provides embodiments of cell culture systems with tunable stiffness containing a biocompatible polydimethyl siloxane substrate and a polyelectrolyte multilayer In certain embodiments, the polyelectrolyte multilayer is present as a plurality of films providing a three dimensional cellular microenvironment within the substrate The tunable stiffness of the cell culture systems as characterized by a Young's modulus can range from about 2 kilopascal (kPa) to about 60 kPa. In certain embodiments, the substrate and the polyelectrolyte multilayer are combined without the addition of any adhesive cell component. Embodiments of cell culture systems with tunable stiffness include systems that mimic a liver research model. These cell culture systems can include one or more of primary human hepatocytes, hepatic stellate cells, cholangiocytes, and/or sinusoidal endothelial cells. These cell culture systems can mimic healthy and disease states of tissues or organs. Embodiments of cell culture systems with tunable stiffness can contain a biocompatible polydimethyl siloxane substrate and a polyelectrolyte multilayer configured to support cells defining a portion of a liver, a heart. an ovary, a uterus, a brain, or a muscle. Embodiments of cell culture systems with tunable stiffness can contain a biocompatible polydimethyl siloxane substrate and a polyelectrolyte multilayer configured to support hepatocytes defining a liver disease research model. The liver disease research model can mimic an alcoholic fatty liver (simple steatosis), alcoholic hepatitis, alcoholic cirrhosis, or hepatocellular carcinoma. The liver disease research model can mimic non-alcoholic fatty liver disease, chronic viral hepatitis, cirrhosis, primary biliary cirrhosis, and primary sclerosing cholangitis. The liver disease research model can be a chronic liver disease (CLD) model.

Embodiments of cell culture systems with tunable stiffness can contain a biocompatible polydimethyl siloxane substrate and a polyelectrolyte multilayer configured to support a plurality of cells defining a human polycystic ovary syndrome research model. Embodiments of cell culture systems with tunable stiffness can contain a biocompatible polydimethyl siloxane substrate and a polyelectrolyte multilayer configured to support a plurality of cells defining a cardiac disease research model. Embodiments of cell culture systems with tunable stiffness can contain a biocompatible polydimethyl siloxane substrate and a polyelectrolyte multilayer configured to support a plurality of cells defining an aging brain research model.

Embodiments of cell culture systems with tunable stiffness can contain a biocompatible polydimethyl siloxane substrate and a polyelectrolyte multilayer configured to support one or more of primary human hepatocytes, hepatic stellate cells, cholangiocytes, and liver sinusoidal endothelial cells. In certain examples, the tunable stiffness of the cell culture system is characterized by a Young's modulus ranging from about 2 kilopascal (kPa) to about 60 kPa. In certain examples, the substrate and the polyelectrolyte multilayer are combined without the addition of any adhesive cell component.

Embodiments of cell culture systems with tunable stiffness can contain a biocompatible polydimethyl siloxane substrate and a polyelectrolyte multilayer configured to support one or more of trophoblasts, endothelial cells, epithelial cells, fibroblasts, and mesenchymal stromal cells. Examples include a cell culture system with tunable stiffness comprising a biocompatible polydimethyl siloxane substrate with a polyelectrolyte multilayer configured to support trophoblasts and defining a human placenta research model mimicking a pregnancy associated disorder. The pregnancy associated disorder can be preeclampsia, intra-uterine growth restriction, gestational diabetes mellitus, or polycystic ovary syndrome.

Other examples of cell culture system with tunable stiffness can include a biocompatible polydimethyl siloxane substrate with a polyelectrolyte multilayer configured to support glial cells and defining a human brain model. The human brain model can mimic aging or a neurological disorder, such as Alzheimer's disease, Parkinson's disease, or traumatic brain injury. Certain examples of cell culture systems can support a plurality of glial cells, such as astrocytes, oligodendrocytes, or microglial cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are included to provide a further understanding of the embodiments of the present disclosure, are incorporated in and constitute a part of this specification. Embodiments are illustrated by way of example and not by way of limitation in the accompanying drawings. According to common practice, the various features of the drawings discussed below are not necessarily drawn to scale. Dimensions of various features and elements in the drawings may be expanded or reduced to more clearly illustrate embodiments of the disclosure.

FIG. 1 is an illustration of the preclinical models used in chronic liver disease (CLD) research providing certain features of the models, their limitations, and their advantages.

FIG. 2 is an illustration of the in vitro bio-engineered adhesive siloxane substrate with tunable stiffness (BEASTS) systems to mimic the stiffness observed in different liver fibrotic stages.

FIGS. 3A-3C present an analysis of the primary human hepatocytes (PHHs) grown in BEASTS (2 kPa, healthy) or collagen matrix for different periods. The data are from three different pooled batches and triplicates for each donor. FIG. 3A is a graphical representation of the levels of urea and albumin production in the BEASTS platform and collagen matrix. FIG. 3B is a set of images showing a higher degree of CLF uptake in PHHs grown on the BEASTS platform compared to collagen substrates, indicating the ability of BEASTS to retain the structural polarity and biliary permeability of PHHs. FIG. 3C is a set of images demonstrating that in the BEASTS platform, PHHs retain ADH and CYP2E1 protein for up to 10 days, comparable to fresh rat liver.

FIG. 4 is a graphical representation of the elastic modulus of PDMs substrates measured using TMS-Pro texture analyzer (n=3).

FIG. 5A is a schema for substrates for patterned co-cultures. FIG. 5B is a graphical representation of the hepatocytes derived EVs (Hep-EVs) concentration for all stiffnesses (2 kPa, 25 kPa, and 55 kPa) using nano-tracking analysis (NTA). FIG. 5C is a graphical representation of the PCR analysis of Hep-EVs which were incubated with hepatic stellate cells (LX-2) on various stiffness. LX-2 cells cultured on tissue culture plate (TCPS) were used as the control. Data represents in mean±SEM, p<0.05 represents statistical significance, * represents p-value <0.05, ** represents p-value <0.001, *** represents p-value<0.0001 using one-way ANOVA, Tukey's multiple comparation test. N=3 for each condition.

FIGS. 6A-6D are directed to RNA seq data analysis for DEG identification and enrichment, and clinical sample analysis of GPR18. FIG. 6A is a volcano plot showing DEGs between two experimental conditions, with genes colored according to their adjusted p-value and log fold change. Red dots represent significantly upregulated genes, while blue dots represent significantly downregulated genes. FIG. 6B is a principal component analysis (PCA) plot of DEGs, showing clustering of samples based on their gene expression profiles. FIG. 6C is a bar plot showing the top enriched Gene Ontology (GO) terms for molecular function, cellular component, and biological process, based on the DEGs identified in the analysis. FIG. 6D is a disease enrichment analysis of DEGs, showing the top enriched disease pathways associated with the identified genes.

FIGS. 7A-7H are directed to the study design and molecular analysis of a PE model with BEASTS platform. FIG. 7A is a schematic diagram of the experimental design, depicting the use of trophoblast cell line (HTR8/SVneo) derived from human first trimester placenta tissue. The BEASTS platform was created using Polydimethylsiloxane gels coated with 10 bilayers of PDAC and SPS polymers via layer-by-layer assembly. Cells were cultured on the BEASTS surfaces with physiologically relevant stiffnesses of 8 kPa mimicking healthy condition and 25 kPa and 55 kPa mimicking preeclampsia and severe preeclampsia conditions, respectively. FIG. 7B is a graphical representation of the stiffness of the BEASTS platform that was assessed by measuring its Young's modulus with a TMSPro texture analyzer. FIG. 7C is a comparative heatmap illustrating gene expression associated with relevant pathways/functions between healthy and PE conditions in clinical samples, as well as 8 kPa, 25 kPa, and 55 kPa conditions mimicking healthy and PE states in the BEASTS platform. FIG. 7D is a graphical representation of the GPR18 gene expression in BEASTS models using PCR analysis. FIG. 7E is a set of four Western Blot analysis revealing GPR18 protein expression in BEASTS. The expression of GPR18 on the trophoblast cells were analyzed in the BEASTS stiffness that recreates healthy placenta stiffness (8 kPa) while the 25 kPa and 55 kPa represents the early stage and late stage preeclamptic environment. FIG. 7F is a graphical representation of the GPR18 protein expression in the 8 kPa, 25 kPa, and 55 kPa BEASTS platforms. FIG. 7G is a set of photographs showing the immunostaining analysis and quantification of GPR18 localization in in the 8 kPa, 25 kPa, and 55 kPa BEASTS platforms. FIG. 7H is a graphical representation of the relative fluorescence intensities in the 8 kPa, 25 kPa, and 55 kPa BEASTS platforms, n=16-22. * indicates p<0.05, ** indicates p<0.01, *** indicates p<0.001, **** indicates p<0.0001.

FIGS. 8A-8G are directed to the impact of substrate stiffness on HTR8 cellular behavior, including morphology, proliferation, and migration. FIG. 8A is a set of representative phase contrast cell images taken over four consecutive days showing the effects of substrate stiffness on HTR8 cell morphology, Scale bar 250 μm. FIG. 8B is a set of photographs showing the immunostaining of actin filaments (green) and cell nuclei (blue) of HTR8 cells after four days of culture on BEASTS surfaces, revealing the influence of substrate stiffness on cell morphology, Scale bar 100 μm. FIG. 8C is a graphical representation of the relative cell area and FIG. 8D is a graphical representation of the cell circularity quantified from actin staining images using NIH Image J, n=15. FIG. 8E is a graphical representation of the cells seeded at 25% confluence, detached at indicated times, and counted to determine the doubling time for each stiffness condition, n=4. FIG. 8F is a set of phase contrast images taken at 24-hour and 36-hour time points post creating a scratch in confluent monolayer of HTR8 cells cultured on 8 kPa, 25 kPa, and 55 kPa BEASTS surfaces, showing the effect of stiffness on cell migration, Scale bar 500 μm. FIG. 8G is a graphical representation of the area migrated by HTR8 cells at 24-hour and 36-hour time points for each experimental condition, n-6-10. * indicates p<0.05, ** indicates p<0.01. *** indicates p<0.001.

FIGS. 9A-9D are directed to the influence of substrate stiffness on cellular oxygen consumption rate and glycolytic activity. FIG. 9A is a graphical representation of the oxygen consumption rate (OCR) via Seahorse mitochondrial stress test and quantification of OCR, where data is normalized to non-mitochondrial oxygen consumption. FIG. 9B is a graphical representation of the OCR parameters—Basal Respiration, ATP Production, Proton Leak, and Maximal Respiratory Capacity—as computed from FIG. 9A, data normalized with respect to 8 kPa, n=4-8. FIG. 9C is a graphical representation of the Extracellular Acidification Rate (ECAR) via Seahorse glycolytic stress test and quantification of ECAR, where data is normalized to non-glycolytic acidification. FIG. 9D is a graphical representation of the ECAR parameters—Glycolysis, Glycolytic Capacity, and Glycolytic Reserve—as computed from FIG. 9C, data normalized with respect to 8 kPa, n=4-9. * indicates p<0.05, ** indicates p<0.01.

FIGS. 10A-10G are directed to the effect of substrate stiffness on oxidative stress, glutathione, Nrf2 activation and sulforaphane treatment in HTR8 cells cultured on BEASTS systems. FIG. 10A is a set of phase contrast and fluorescent microscope images depicting ROS intensities after staining cells with CM-H2DCFDA dye (green). Scale bar 200 μm. FIG. 10B is a graphical representation of the fluorescent intensities using NIH Image J, n=4. FIG. 10C is a graphical representation of reduced glutathione (GSH) and oxidized glutathione (GSSG) concentrations using Promega GSH/GSSG-glo assay, n-3-4. FIG. 10D is a set of immunofluorescence images displaying Nrf2 (red) and cell nuclei (blue) to assess Nrf2 nuclear translocation. Scale bar 100 μm. FIG. 10E is a graphical representation of the nucleus/cytoplasm Nrf2 intensities, n=15-18. FIG. 10F is a graphical representation of the relative gene expression levels of Nrf2-related genes in HTR8 cells cultured on 8 kPa, 25 kPa, and 55 kPa BEASTS surfaces for four days, measured via RT-PCR and normalized with respect to the 8 kPa condition, n=4-6.

FIG. 10G is a graphical representation of the effect of sulforaphane treatment on gene expression levels of Nrf2-related genes in HTR8 cells cultured on 8 kPa, 25 kPa, and 55 kPa BEASTS surfaces, as determined by RT-PCR. * indicates p<0.05, ** indicates p<0.01, *** indicates p<0.001, * indicates p<0.0001.

FIG. 11 is a schematic representation of HTR8 cell behavior in the BEASTS model and its relevance to preeclampsia. The BEASTS model closely mimics cellular behavior and functions observed in preeclampsia (PE) using HTR8 cells. On stiffer substrates that replicate the conditions in PE, the oxidative stress levels are heightened, and the glutathione concentration is decreased. Metabolic analyses reveal a substantial upregulation of mitochondrial respiration, while glycolysis function is reduced as substrate stiffness increases. The translocation of Nrf2 is diminished under higher stiffness conditions, indicating an impaired antioxidant response. Nrf2 activation is enhanced upon sulforaphane treatment, as demonstrated by the upregulation of relevant gene expression. This figure highlights the potential therapeutic effects of sulforaphane in mitigating PE-associated cellular dysfunctions and improving antioxidant response in the context of the BEASTS model.

FIGS. 12A-12F are directed to analysis of the aging patterns in the brain transcriptome. FIG. 12A is a PCA plot of prefrontal cortex brain tissues in ≥40 and <40 years. The total number of genes: 1023.≥40, n-290.<40, n-24. Data was standardized and PCs were selected based on parallel analysis. FIG. 12B is a set of selected gene ontology analysis of differentially expressed genes (DEGs) of molecular function (10,819 input genes, 4,181 annotated genes before applying any cutoffs, and 19,869 genes in the enriched category) (left panel), cellular component (11,003 input genes, 1,877 annotated genes before applying any cutoffs, and 20,659 genes in the enriched category) (middle panel), and biological process (10,929 input genes, 15,199 annotated genes before applying any cutoffs, and 20,669 genes in the enriched category) (right panel). Significance was calculated based on Welch's t-test followed by the Bonferroni multiple correction method.

FIG. 12C is a bubble plot showing the disease enrichment analysis of ≥40 and <40 years tissue samples (9,826 input genes, 33,367 annotated genes before applying any cutoffs, and 19,381 genes in the enriched category). Significance was calculated based on the Bonferroni multiple correction method. FIG. 12D is a Volcano plot of the differentially expressed genes (DEGs) normalized to log fold change in >40 and <40 prefrontal cortex samples. Highlighted genes related to priming and activation, MI inflammatory profile, mechanosensors & mechanosensing, and antioxidant & response elements were grouped by color. Total number of DEG genes: 34,277. Total number of genes: 39,282. Significance was calculated by Welch's t-test.≥40, n=290.<40, n=24. FIG. 12E is a set of two representative heatmaps of DEGs. Left panel provides≥40 and <40 prefrontal cortex samples grouped by function (Significance was calculated by Welch's t-test). Right panel provides HMC3 microglia grown on 2, 8, and 25 kPa. Significance was calculated by Student's t-test. Significant=p<0.05; Not significant=p>0.05. n=6 in triplicate. FIG. 12F is a set of three representative heatmaps of fibrous ECM genes (left panel), laminins (middle panel), and brain ECM scaffolding proteins (right panel). Significance was calculated by Welch's t-test. Data represents mean±SEM.≥40, n=290.<40, n=24.

FIGS. 13A-13F are directed to the creation of a biomimetic platform to better represent physiological and pathological conditions. FIG. 13A is a graphical representation of the shear modulus of aged brain tissue in Homo sapiens studies. Neurodegenerative diseases, psychotic conditions, and traumatic brain injury values were excluded. FIG. 13B is an illustration of the BEASTS platform to study microglia on stiffness. Created in Biorender.com. FIG. 13C shows that the combination of Slygard 527 forms the soft matrix with increasing Slygard 184 crosslinker creating increasing stiffness. Data represents mean±SEM of three replicates. FIG. 13D is a set of phase contrast images of microglial cell line HMC3 after 11 days in culture on BEASTS surfaces. Left panels: 2 kPa, Middle panels: 8 kPa, and Right panels: 25 kPa show the systems at Day 1 (top left, middle, and right panels) and Day 11 (bottom left, middle, and right panels), respectively (Scale Bar 200 μm). FIG. 13E is a set of Z-stacked actin-labeled images counterstained with DAPI in HMC3 cells grown on varying stiffness. Scale Bar=50 μm. FIG. 13F is a graphical representation of the Actin+DAPI-fluorescence intensity. Significance was calculated using an ordinary one-way ANOVA with Tukey's multiple comparisons using statistical hypothesis testing. n-4. * indicates p<0.05, *** indicates p<0.001. Data represents mean #SEM.

FIGS. 14A-14G demonstrate that stiffness regulates microglial activation and priming. FIG. 14A is a graphical representation of the proliferation and growth of HMC3 (right) microglia as assessed by MTT. Significance is measured in comparison to 2 kPa at the indicated time point and tested with multiple t-tests using the Bonferroni-Dunn method. Symbols $=25>2 kPa, *=8 kPa>2 kPa. FIG. 14B is a graphical representation of the microglial growth and doubling rate of HMC3 cells. Significance is measured in comparison to 2 kPa at the indicated time point and tested with multiple t-tests using the Bonferroni-Dunn method. Symbols $=25>2 kPa, *=8 kPa>2 kPa. The cells were seeded at the same density (75,000). Scratch wound closure is monitored over time in cells grown on stiffness. FIG. 14C is a set of representative brightfield images demonstrating that increasing stiffness increases migration speed. Edges were detected using a macro plugin in ImageJ. Scale bar=400 μm. FIG. 14D is a graphical representation of the wound closure. Wound closure was calculated as the difference between the final and initial wound width over time. FIG. 14E is a graphical representation of the LDH release in HMC3 microglia. The percentage was normalized from 100% release by 0.1% Triton X-100 with luminescence values. Significance was calculated using an ordinary one-way ANOVA. Data presented * indicates p<0.05, **** indicates p<0.0001. n=6 in triplicate. FIG. 14F is a graphical representation of the urea secretion (mg/dL) in HMC3 microglia. Significance was calculated using an ordinary one-way ANOVA with Tukey's multiple comparisons using statistical hypothesis testing. * indicates p<0.05, *** indicates p<0.001. n=4. FIG. 14G is a graphical representation of the IL6 secretion analyte (pg/mL) as detected by ELISA in HMC3 microglia. Significance was calculated using an ordinary one-way ANOVA with Tukey's multiple comparisons using statistical hypothesis testing. **** indicates p<0.0001. n=6.

FIGS. 15A-15G demonstrate that stiffness regulates microglial metabolic reprogramming. FIGS. 15A and 15B are graphical representations of representative OCR profile of HMC3 microglia. During aging, microglia is under inflammatory activation state (M1) and microglia shifts their energy metabolism from oxidative phosphorylation to glycolysis. To measure metabolic flux in real-time, the Seahorse XFe24 analyzers were utilized to measure the oxygen consumption rate (OCR) in microglia grown on soft (young environment) and stiff (aged environment) tissue mimics. The microglia grown on 2 kPa utilize the electron transport chain (ETC), whereas microglia grown on 8 and 25 kPa have a lower overall OCR profile (FIG. 15B). The rates were quantified and significant decreases in 8 and 25 kPa in basal respiration, ATP-linked respiration, proton leak, maximal respiration, reserve capacity, and non-mitochondrial respiration (FIG. 15B). In contrast, microglia grown on 2 kPa exhibited a lower ECAR profile than 8 or 25 kPa (FIG. 15B) and significant changes between microglial polarization. Strikingly, when the changes were quantified, a grated increase was found in the measurements of glycolysis, glycolytic capacity, glycolytic reserve, and an overall increase in non-glycolytic acidification (FIG. 15B). The changes in microglial metabolism indicate that the BEAST platform recreates age-related metabolic defects observed in inflammatory microglia, with specific disease-related phenotypes. Measures of mitochondrial oxidative phosphorylation (OXPHOS) capacity (using Seahorse) in HMC3 microglial cells after eleven days grown on stiffness including: basal respiration, ATP-linked respiration, proton leak, maximal respiration, reserve capacity, and non-mitochondrial respiration. Significance is measured in comparison to 2 kPa and tested with multiple t-tests using the Bonferroni-Dunn method. * indicates p<0.05, ** indicates p<0.01, *** indicates p<0.001, **** indicates p<0.0001. n=4 in duplicate. FIGS. 15C and 15D are graphical representations of representative ECAR profile of HMC3 microglia. Measures of glycolysis capacity (using Seahorse) in HMC3 microglial cells after eleven days grown on stiffness including glycolysis, glycolytic capacity, glycolytic reserve, and non-glycolytic acidification. Significance is measured in comparison to 2 kPa and tested with multiple t-tests using the Bonferroni-Dunn method. * indicates p<0.05, ** indicates p<0.01, *** indicates p<0.001, **** indicates p<0.0001. n=4 in duplicate. The Seahorse XFe24 analyzers were further used to measure the extracellular acidification rate (ECAR) in microglia grown on soft and stiff tissue mimics. Microglia grown on 2 kPa exhibited a lower ECAR profile than 8 or 25 kPa (FIG. 15D) and significant changes between microglial polarization. Strikingly, when the changes were quantified, a grated increase was found in the measurements of glycolysis, glycolytic capacity, glycolytic reserve, and an overall increase in non-glycolytic acidification (FIG. 15D). The changes in the microglial metabolism recreate the glycolytic changes in microglia during aging and neurological disease-related phenotypes. FIG. 15E is a set of images showing the neutral lipid accumulation in HMC3 microglia grown on 2, 8, and 25 kPa through fluorescent staining of BODIPY 493/503 and counterstained with DAPI. Scale Bar=200 μm. FIG. 15F is a graphical representation of the neutral lipid accumulation in HMC3 microglia using the BODIPY+DAPI+signal. Significance is measured in comparison to 2 kPa and tested using an ordinary one-way ANOVA with Tukey's multiple comparisons using statistical hypothesis testing. * indicates p<0.05, ** indicates p<0.01, *** indicates p<0.001, * indicates p<0.0001. n=9. FIG. 15G is a graphical representation of the results from the cholesterol profile quantification assay in HMC3 on differing stiffness including intracellular cholesterol esters, intracellular free cholesterol, and total cholesterol esters. Significance is measured in comparison to 2 kPa and tested with multiple t-tests using the Bonferroni-Dunn method. * indicates p<0.05, ** indicates p<0.01, *** indicates p<0.001, **** indicates p<0.0001. n=5.

FIGS. 16A-16F demonstrate that stiffness regulates oxidative stress and drives inflammation. FIG. 16A is a set of images to show the total cellular ROS levels when imaged after eleven days on stiffness by the H2DCFDA dye. (Scale Bar 200 μm). FIG. 16B is a graphical representation of the total intracellular ROS by relative fluorescence intensity. Significance is measured in comparison to 2 kPa and tested using an ordinary one-way ANOVA with Tukey's multiple comparisons using statistical hypothesis testing. * indicates p<0.05, ** indicates p<0.01, *** indicates p<0.001, **** indicates p<0.0001.2 kPa, n=10.8 kPa, n=12.25 kPa, n=13. FIGS. 16C and 16D are graphical representations of the redox status and GSSG quantification (mM/microglia) of HMC3 microglia grown on stiffness. Significance is measured in comparison to 2 kPa and tested with multiple t-tests using the Bonferroni-Dunn method. * indicates p<0.05, ** indicates p<0.01, *** indicates p<0.001, **** indicates p<0.0001. n=6. FIG. 16E is a graphical representation of the impact of stiffness on microglial caspase-1 activation and non-canonical caspase activity. Intracellular caspase-1 activity was measured by a bioluminescent assay of microglia. Detection of the specificity of caspase-1 activity was confirmed by a selected caspase-1 inhibitor (Ac-YVAD-CHO, 1 μM). Significance is measured in comparison to 2 kPa and tested using an ordinary one-way ANOVA with Tukey's multiple comparisons using statistical hypothesis testing. * indicates p<0.05, ** indicates p<0.01, *** indicates p<0.001, indicates p<0.0001. n=9. FIG. 16F is a graphical representation of the increased concentrations of excreted 8-oxo-2′-deoxyguanosine (8-OHdG) with increased stiffness. Significance is measured in comparison to 2 kPa and tested using an ordinary one-way ANOVA with Tukey's multiple comparisons using statistical hypothesis testing. * indicates p<0.05, ** indicates p<0.01, *** indicates p<0.001, **** indicates p<0.0001. n=6.

FIGS. 17A-17G demonstrate that N-acetyl-cysteine ameliorates stiffness-induced redox dysfunction. FIG. 17A is a set of phase contrast images of microglial cell line HMC3 pre-treated with 0.25 mM NAC after 1 day of seeding and 11 days in culture on BEASTS surfaces. Scale bar 200 μm. FIG. 17B is a set of images showing the total cellular ROS levels that were imaged after eleven days of pretreatment of 0.25 mM NAC on stiffness by the H2DCFDA dye. FIG. 17C is a graphical representation of the total intracellular ROS upon quantification of the NAC pre-treatment groups only by relative fluorescence intensity. Significance is measured in comparison to 2 kPa, no NAC pretreatment, and tested using an ordinary one-way ANOVA with Tukey's multiple comparisons using statistical hypothesis testing. * indicates p<0.05, ** indicates p<0.01, *** indicates p<0.001, **** indicates p<0.0001. n=5. FIG. 17D is a graphical representation of the total intracellular ROS by relative fluorescence intensity in microglia grown with and without 0.25 mM NAC pretreatment and on varying stiffnesses. Significance is measured in comparison to 2 kPa, no NAC pretreatment, and tested using an ordinary one-way ANOVA with Tukey's multiple comparisons using statistical hypothesis testing. * indicates p<0.05, ** indicates p<0.01, *** indicates p<0.001, indicates p<0.0001.2 kPa, n=10.8 kPa, n=12.25 kPa, n=13. FIG. 17E is representative heatmap of microglia without NAC pretreatment (left panel) and with 0.25 mM NAC pretreatment (right panel). Data was grouped by the normalized fold change in groups of priming and activation, MI inflammatory profile, mechanosensors and mechanosensing receptors, and antioxidant defense and antioxidant response genes based on RT-qPCR. Significance was calculated by Student's t-test. Significant=p<0.05; Not significant=p>0.05. n=6 in triplicate. The impact of stiffness and pretreatment of 0.25 mM NAC on microglial caspase-1 activation and non-canonical caspase activity. FIG. 17F is a graphical representation of the intracellular caspase-1 activity as measured by a bioluminescent assay of microglia. FIG. 17F is a graphical representation of the specificity of caspase-1 activity as confirmed by a selected caspase-1 inhibitor (Ac-YVAD-CHO, 1 M). Significance is measured in comparison to 2 kPa with no NAC pretreatment and tested using an ordinary one-way ANOVA with Tukey's multiple comparisons using statistical hypothesis testing. * indicates p<0.05, ** indicates p<0.01, *** indicates p<0.001, **** indicates p<0.0001. n-9.

FIG. 18 is an illustration of the stiffness-regulated response in microglia, created using Biorender.com.

### DETAILED DESCRIPTION

The disclosure herein provides embodiments of cell culture systems with tunable stiffness containing a biocompatible polydimethyl siloxane substrate and a polyelectrolyte multilayer. Also provided here are methods of making and using these systems as research and product development models for different tissue and organ systems.

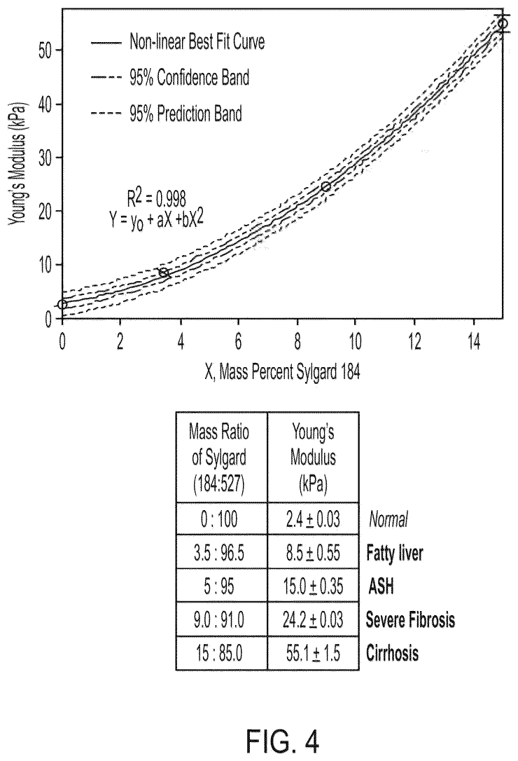

Embodiments of the cell culture systems with tunable stiffness can include a variety of polydimethyl siloxane (PDMS) formulations to form the substrate. In certain embodiments, the substrate is a PDMS blend. For example, the substrate can include different formulations of blends of Sylgard™ 184 and Sylgard™ 527. Embodiments can include substrates formed by combination of Sylgard™ 184 and Sylgard™ 527 at weight percent ratios ranging from about 0:100 to about 20:80. In certain embodiments, the weight percent ratio of Sylgard™ 184 and Sylgard™ 527 can be 2:98, 3.5:96.5, 5:95, 7.5:92.5, 9:91, 15:85, or 17.5:82.5. Embodiments include PDMS blends of specific ratios to achieve a stiffness characterized by a Young's modulus ranging from about 2 kilopascal (kPa) to about 60 kPa. In certain embodiments, a substrate with Sylgard™ 184 has a stiffness of about 2.4+0.03 kPa. In certain embodiments, a substrate from a blend of Sylgard™ 184 and Sylgard™ 527 at a weight percent ratio of about 3.5:96.5 has a stiffness of about 8.5+0.55 kPa. In certain embodiments, a substrate from a blend of Sylgard™ 184 and Sylgard™ 527 at a weight percent ratio of 5:95 has a stiffness of about 15+0.35 kPa. In certain embodiments, a substrate from a blend of Sylgard™ 184 and Sylgard™ 527 at a weight percent ratio of 9:91 has a stiffness of about 24.2+0.03 kPa. In certain embodiments, a substrate from a blend of Sylgard™ 184 and Sylgard™ 527 at a weight percent ratio of 15:85 has a stiffness of about 55.1=1.5 kPa. Depending on the stiffness needs, other PDMS compounds and blends can be used to form the substrate. For example, the ratio of the Sylgard™ 184 and Sylgard™ 527 is used to modulate the stiffness to develop certain liver disease models, as shown in FIG. 4. By varying the weight ratio, one can obtain a representative stiffness of any tissue/organ in its healthy state or representing the disease environment.

As used herein, the term “substrate” refers to a two or three dimensional environment that can support a cell, a cell culture, a cell culture material, or on which a cellular process can occur. In certain embodiments, a “substrate” is substantially a solid substance providing support to a cell, a cell culture, a cell culture material, or a cellular process. Another material can be deposited in or combined with the substrate to form the cell culture system. In certain embodiments, the polyelectrolyte multilayer is present as a plurality of films providing a three dimensional cellular microenvironment within the substrate. The substrates can be patterned, e.g., are patterned in 3D with PEM.

In certain embodiments, the PDMS substrate and the polyelectrolyte multilayer are combined without the addition of any adhesive cell component. One of the challenges with existing platforms is the dependence on protein coating for cell adhesion. Majority of stiffness studies use collagen and matrigels as the matrix. The animal-based source and lack of information on the composition is a major limitation with the use of these gels as they might cause non-physiological responses from the cells. Polyacrylamide gels, commonly used synthetic biomaterials for stiffness studies, are limited due to need for covalent crosslinking of proteins using harsh chemicals for cell adhesion and elastic creasing instability of the softer polyacrylamide gel resulting in non-specific cell behavior. Also, protein coating in different stiffness remodels affect the protein conformation and results in different binding domains presented to the cells. This causes non-biological response from the cells and will not mimic the disease response of the cells. The BEASTS platform utilizes synthetic polymer films to coat the PDMS substrate, which helps cells attach and perform similar or better than protein coatings or other adhesive components.

These cell culture systems can mimic healthy and disease states of tissues or organs. Embodiments of cell culture systems with tunable stiffness can contain a biocompatible polydimethyl siloxane substrate and a polyelectrolyte multilayer configured to support a plurality of cell types defining a portion of a liver, a heart, an ovary, a uterus, a brain, or a muscle.

Embodiments of cell culture systems with tunable stiffness include systems that mimic a liver research model. These cell culture systems can include one or more of primary human hepatocytes, hepatic stellate cells, cholangiocytes, and/or sinusoidal endothelial cells. In certain embodiments, these cell culture systems can include one or more of primary human hepatocytes; primary stellate cells, macrophages, cholangiocytes, liver sinusoidal endothelial cells, kuppfer cells, and fibroblasts. Embodiments of cell culture systems with tunable stiffness can contain a biocompatible polydimethyl siloxane substrate and a polyelectrolyte multilayer configured to support a plurality of cells defining a liver disease research model. The liver disease research model can mimic a fatty liver (simple steatosis), hepatitis, cirrhosis, hepatocellular carcinoma, non-alcoholic fatty liver disease, chronic viral hepatitis, cirrhosis, primary biliary cirrhosis, and primary sclerosing cholangitis. The liver disease research model can be a chronic liver disease model.

Embodiments of cell culture systems with tunable stiffness can contain a biocompatible polydimethyl siloxane substrate and a polyelectrolyte multilayer configured to support a plurality of cells defining a human polycystic ovary syndrome or preeclampsia research model. Embodiments of cell culture systems with tunable stiffness can contain a biocompatible polydimethyl siloxane substrate and a polyelectrolyte multilayer configured to support a plurality of cells defining a cardiac disease research model. Embodiments of cell culture systems with tunable stiffness can contain a biocompatible polydimethyl siloxane substrate and a polyelectrolyte multilayer configured to support a plurality of cells involved in aging, cancer, or other neurodegenerative diseases.

Embodiments of cell culture systems with tunable stiffness can contain a biocompatible polydimethyl siloxane substrate and a polyelectrolyte multilayer configured to support one or more of primary human hepatocytes, hepatic stellate cells, cholangiocytes, and liver sinusoidal endothelial cells. In certain examples, the tunable stiffness of the cell culture system is characterized by a Young's modulus ranging from about 2 kilopascal (kPa) to about 60 kPa. In certain examples, the substrate and the polyelectrolyte multilayer are combined without the addition of any adhesive cell component.

Embodiments of cell culture systems with tunable stiffness can contain a biocompatible polydimethyl siloxane substrate and a polyelectrolyte multilayer configured to support one or more of trophoblasts, endothelial cells, epithelial cells, fibroblasts, and mesenchymal stromal cells. Examples include a cell culture system with tunable stiffness comprising a biocompatible polydimethyl siloxane substrate with a polyelectrolyte multilayer configured to support trophoblasts and defining a human placenta research model mimicking a pregnancy associated disorder. The pregnancy associated disorder can be preeclampsia, intra-uterine growth restriction, gestational diabetes mellitus, or polycystic ovary syndrome. In certain examples, the tunable stiffness of the cell culture system is characterized by a Young's modulus ranging from about 2 kilopascal (kPa) to about 60 kPa. In certain examples, the substrate and the polyelectrolyte multilayer are combined without the addition of any adhesive cell component.

Other examples of cell culture system with tunable stiffness can include a biocompatible polydimethyl siloxane substrate with a polyelectrolyte multilayer configured to support glial cells and defining a human brain model. The human brain model can mimic aging or a neurological disorder, such as Alzheimer's disease, Parkinson's disease, or traumatic brain injury. Certain examples of cell culture systems can support a plurality of glial cells, such as astrocytes, oligodendrocytes, or microglial cells. In certain examples, the tunable stiffness of the cell culture system is characterized by a Young's modulus ranging from about 2 kilopascal (kPa) to about 60 kPa. In certain examples, the substrate and the polyelectrolyte multilayer are combined without the addition of any adhesive cell component.

In some embodiments, the polyelectrolyte multilayer contains one or more independently selected polycationic polymers. In some embodiments, the polyelectrolyte multilayer contains one polycationic polymer. In some embodiments, each of the polycationic polymers is independently selected from the group consisting of poly-D-lysine (PDL), poly-L-lysine (PLL), poly(diallyl dimethylammonium chloride) (PDAC), linear poly(ethylene imine) (LPEI), poly(allyl-amine hydrochloride) (PAH), chitosan (CHI), a poly(β-amino ester), and polyarginine. In some embodiments, the polyelectrolyte multilayer contains one or more independently selected polyanionic polymers. In some embodiments, the polyelectrolyte multilayer contains one polyanionic polymer. In some embodiments, each of the polyanionic polymers is one or more of poly(sodium styrene sulfonate) (SPS), poly(anetholesulfonic acid) (PAS), poly(acrylic acid) (PAA), poly(sodium vinylsulfonate) (PVS), graphene oxide (GO), dextran sulfate, collagen, and hyaluronic acid (HA). In some embodiments, at least one of the polycationic polymers is poly-L-lysine (PLL) and at least one of the polyanionic polymers is poly(sodium styrene sulfonate) (SPS). In some embodiments, the ratio of PLL/SPS is from about 1 to about 2. In some embodiments, the ratio of PLL/SPS is from about 1 to about 1.75. In some embodiments, the ratio of PLL/SPS is from about 1 to about 1.5. In some embodiments, the polyelectrolyte multilayer contains from about 2 to about 10 monolayers of PLL. In some embodiments, the polyelectrolyte multilayer contains from about 2 to about 8 monolayers of PLL. In some embodiments, the polyelectrolyte multilayer contains from about 2 to about 6 monolayers of PLL. In some embodiments, the polyelectrolyte multilayer contains from about 3 to about 5 monolayers of PLL. In some embodiments, the polyelectrolyte multilayer contains from about 2 to about 10 monolayers of SPS In some embodiments, the polyelectrolyte multilayer contains from about 2 to about 8 monolayers of SPS. In some embodiments, the polyelectrolyte multilayer contains from about 2 to about 6 monolayers of SPS. In some embodiments, the polyelectrolyte multilayer contains from about 3 to about S monolayers of SPS. In some embodiments, the polyelectrolyte multilayer contains about 5 monolayers of PLL and about 4 monolayers of SPS.

Embodiments include methods of making the cell culture systems with tunable stiffness can contain a PDMS substrate and a PEM. One such method includes subjecting the PDMS surfaces to a plasma cleaner in the presence of oxygen in a plasma chamber. Following the plasma treatment, the PDMS surfaces were coated with PEMs to create PEM-coated PDMS. In certain embodiments, a slide stainer is used to coat the substrate with PEMs. In certain embodiments, PDAC and SPS are strong polyelectrolytes, resulting in smooth, homogeneous and stable PEM films suitable for cellular studies and used for several of the tissue/organ models to coat the PDMS surfaces. To form the first bilayer, the PDMS substrates were immersed in a polycation solution (PDAC). Following two sets of rinses with water, the PDMS substrates subsequently placed in a polyanion (SPS) solution and this PEM was allowed to deposit. Afterwards, PDMS substrates were rinsed again. This process was repeated to build multiple layers, as required for the application. Before seeding cells, PDMS substrates were placed into tissue culture plates and exposed to UV overnight to sterilize the surfaces.

In the following description, numerous details are set forth in order to provide a thorough understanding of the various embodiments. In other instances, well-known processes, devices, and systems may not been described in particular detail in order not to unnecessarily obscure the various embodiments. Additionally, illustrations of the various embodiments may omit certain features or details in order to not obscure the various embodiments.

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “about” or “approximately” are defined as being close to as understood by one of ordinary skill in the art. In one non-limiting embodiment, the terms are defined to be within 10%, preferably within 5%, more preferably within 1%, and most preferably within 0.5%. The term “plurality” as used herein refers to two or more items or components. The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

### BEASTS Platform in Chronic Liver Disease

Chronic liver diseases (CLD) affect over 35 million Americans with estimated health care costs of $10 billion per year with no FDA-approved interventions. CLD encompasses a broad spectrum of conditions, including alcoholic fatty liver (simple steatosis), alcoholic hepatitis, alcoholic cirrhosis, non-alcoholic fatty liver disease, chronic viral hepatitis, cirrhosis, primary biliary cirrhosis, primary sclerosing cholangitis, and hepatocellular carcinoma. No effective treatments for CLD currently exist but for reducing alcohol consumption or liver transplantation. The current paucity of clinically relevant experimental models impedes any effort to identify CLD prognostic indicators and potential effective treatment options.

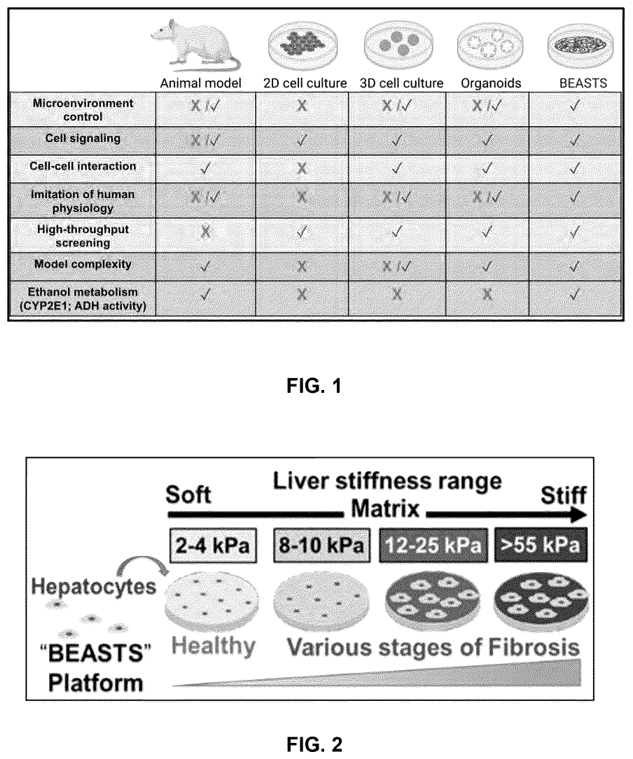

Currently, CLD diagnosis relies on a combination of clinical and laboratory findings. To improve diagnostics and develop effective treatments, it is important to develop faithful models of the human liver responses to disease-relevant challenges (FIG. 1). FIG. 1 is an illustration of the preclinical models used in CLD research providing certain features of the models, their limitations, and their advantages.

In certain embodiments, the BEASTS platform is based on a polydimethyl siloxane (PDMS) substrate in combination with a polyelectrolyte multilayer (PEM) film-coating technology to engineer mechanically tunable substrates mimicking physiologic and pathologic stiffness. Embodiments of the systems have the ability to tune the PDMS stiffness and facilitate cell adhesion without the aid of adhesive proteins (FIG. 2).

Disclosed here are novel biomimetic liver model systems closely mimicking the hepatic environment in physiologic and pathological conditions enabled by microtechnology and the incorporation of primary human hepatocytes (PHHs). Ultimately, this disruptive technology will enable the rapid screening of pharmacological compounds for beneficial or detrimental effects on CLD and for the detection of pharmacogenetic interactions. Embodiments include novel biomimetic BEASTS platform, which enables preserving primary human hepatocytes (PHHs) including liver-specific synthetic functions (urea and albumin production and bile acid uptake), maintaining alcohol dehydrogenase (ADH) and CYP2E1 activity for about 10 days. BEASTS platform uniquely recreates the physiologic (2 kPa) and pathologic liver stiffness at various stages of CLD (8, 15, 25, 55 kPa), which mimics fibrosis in CLD patients that is lacking in current animal models. The BEASTS platform has been demonstrated to provide an ideal environment to maintain the expression of key ethanol metabolizing enzymes (CYP2E1 and ADH) in PHHs for extended periods of time (up to 10 days). Such versatility allows for the development of a chronic liver injury model using PHHs. The BEASTS platform demonstrated that: (1) stiffness induces decreased hepatic urea, albumin production, and expression of drug transporter gene and epithelial cell phenotype marker, hepatocyte nuclear factor 4 alpha (HNF4a) in PHHs; (2) PHHs on fibrotic stiffness inhibits ATP production and maximal respiration and increases glycolysis and glycolytic capacity akin to metabolic changes observed in CLD patients; (3) PHHs cultured on fibrotic stiffness shows increased ROS; and (4) decreased reduced glutathione (GSH) levels culminating in apoptosis akin to CLD patients.

Embodiments of the BEAST systems have significant impact in the treatment of CLD, including development of novel therapies that maintain various liver cell function in CLD patients with fibrotic liver disease targeting stiffness, high-throughput screening of new therapeutic targets, and novel biochemical markers that regulates liver function during CLD progression. Ultimately this disruptive technology enables the rapid screening of pharmacological compounds for beneficial or detrimental effects on CLD and for the detection of pharmacogenetic interactions.

Alcoholic liver disease (ALD) is the most prevalent chronic liver disease. No effective treatments for ALD currently exist except for reducing alcohol consumption or liver transplantation. The current paucity of clinically relevant experimental models impedes any effort to identify ALD prognostic indicators and potential effective treatment options. For example, any clinical testing on ALD patients is not feasible due to ethical and regulatory constraints. Interrogation of the complex molecular and dynamic changes in ALD have thus far only been possible using animal models. However, animal models do not lend themselves to disease staging or high-throughput approaches and frequently deviate from humans in key metabolic features, thus greatly impeding efforts to discover treatments for ALD. Embodiments of the BEASTS platform have been demonstrated to provide an ideal environment to maintain the expression of key ethanol metabolizing enzymes (CYP2E1 and ADH) in PHHs for extended periods of time. All the preliminary data were generated from PHHs from at least three different pooled batches (triplicates for each donor). The BEAST systems allow preserving the normal functions of non-parenchymal cells, such as hepatic stellate cells (HSCs) and sinusoidal endothelial cells for a substantially longer period in culture conditions. The BEAST systems allow use of these cells for chronic ethanol treatment. Furthermore, manipulation of the gel rigidity, which allows mimicking liver stiffness induced by fibrosis, further aids in fibrosis-targeted investigations. The BEAST systems can enable predictive modeling of human responses to perturbations more accurately than current preclinical models used by ALD researchers. Thus, methods that utilize the BEAST systems address a significant gap in the need for biomimetic cellular models integrating PHHs and maintaining key ethanol-metabolizing features.

### BEASTS Platform Retains PHH Functions Critical for Recreating the ALD Phenotype

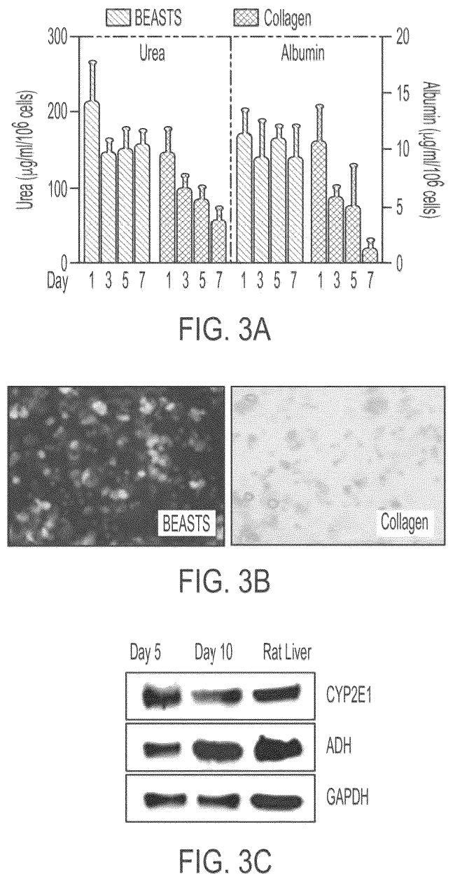

PHHs are an excellent model system for ALD studies. Yet, when in culture (within a few days post-seeding), the cells begin to rapidly lose their functional ability, including ADH and CYP2E1 expression, which are critical markers for hepatocyte alcohol metabolism and ROS processing. In comparison to the gold standard (collagen) where PHHs loses its phenotype in 3 days, the BEASTS platform (2 kPa) retains levels of urea and albumin production up to 2 weeks (FIG. 3A), indicating enduring liver-specific synthetic functions. The uptake of bile acids tagged with the fluorophore cholyl-lysyl-fluorescein (CLF) has been used as a surrogate to assess hepatic bile acid transport. A higher degree of CLF uptake was noted in PHHs grown on the BEASTS platform up to 7 days compared to collagen substrates, indicating the ability of BEASTS to retain the structural polarity and biliary permeability of PHHs (FIG. 3B). None of the current commercial liver models have demonstrated the ability of the platform to retain and maintain two key regulators of alcohol metabolism in hepatocytes, namely, CYP2E1 and ADH. In the BEASTS platform, PHHs retain ADH and CYP2E1 protein for up to 10 days, comparable to fresh rat liver (FIG. 3C).

Maintaining the quiescence of stellate cells is a challenging task as they begin differentiating as soon as they are plated on petri dishes. This is a rate-limiting factor to investigating the mechanism(s) of HSC activation. As the BEASTS platform provides the freedom to control the stiffness, therefore one can maintain the quiescence/fibrogenic phenotype of HSCs.

### BEASTS Platform Retains Primary Human Hepatocytes Functions Critical for Recreating the CLD Phenotype:

PHHs are an excellent model system for ALD studies. Yet, when in culture (within a few days post-seeding), they begin to rapidly lose their functional ability, including ADH and CYP2E1 expression, which are critical markers for hepatocyte metabolism and oxidative stress processing. In comparison to the gold standard (collagen) where PHHs loses its phenotype in 3 days, the BEASTS platform (2 kPa) retains levels of urea and albumin production up to 2 weeks (FIG. 3A), indicating enduring liver-specific synthetic functions. The uptake of bile acids tagged with the fluorophore cholyl-lysyl-fluorescein (CLF) has been used as a surrogate to assess hepatic bile acid transport. A higher degree of CLF uptake was noted in PHHs grown on the BEASTS platform up to 7 days compared to collagen substrates, indicating the ability of BEASTS to retain the structural polarity and biliary permeability of PHHs (FIG. 3B). None of the current commercial liver models have demonstrated the ability of the platform to retain and maintain two key regulators of alcohol metabolism in hepatocytes, namely, CYP2E1 and ADH. In the BEASTS platform, PHHs retain ADH and CYP2E1 protein for up to 10 days, comparable to fresh rat liver (FIG. 3C).

Engineering Substrates with Physiologic and Pathologic Stiffness.

To determine the impact of physiologic and pathologic stiffness on cell function, an in vitro platform was engineered to model specific snapshots of increasing pathologic stiffness (FIG. 4). Specifically, five platforms of different stiffnesses were created to model distinct stages of fibrosis based on Fibroscan (transient elastography) measurements of ALD patients: healthy (2 kPa-soft), fatty liver (8 kPa), AH (15 kPa), fibrosis (25 kPa), and cirrhosis (55 kPa-stiff). These stiffness values mimic the stress/pressure the hepatocytes are under during various stages of ALD/fibrosis in the human liver. Using the BEASTS platform, the stiffness of the matrix was tuned without changing the overall property of the material and without adhesive ligands.

A Multicellular Organized Platform with PHH and Stellate Cells to Create a Fibrosis/CLD-On-a-Dish Model.

Cell-cell interactions play a fundamental role in liver function and have been implicated in adult liver physiology and pathophysiology (i.e., cirrhosis, and response to injury). Liver diseases are perpetuated by the orchestration of hepatocytes and other hepatic non-parenchymal cells (NPC). Growing evidence shows that under both physiological and pathological conditions, several hepatocyte functions are regulated by neighboring NPCs. Despite extensive work in addressing the role of hepatocytes interaction with NPCs in regulating hepatic functions, the impact of increasing liver stiffness during liver diseases in modulating cell-cell interactions and hepatocyte phenotype in vitro remains unelucidated. Direct and indirect co-culture systems have been developed to further recreate the interaction of liver cells during CLD. These platforms/technologies primarily focused on improving hepatic cellular functions and do not recreate the interaction of hepatocytes and NPCs in a healthy and disease like environments. The BEASTS platform provides a controlled microenvironment that can be used to examine the interaction of hepatocytes and NPCs in vitro at healthy (2.36±0.04 kPa), early disease stage (24.20±0.03 kPa) and severe case of liver disease (54.98±2.15 kPa). This technology is ideally suited for rapid testing medical devices in the future for drug testing purposes specially to identify toxicity for liver and potentially provide a cure to CLDs.

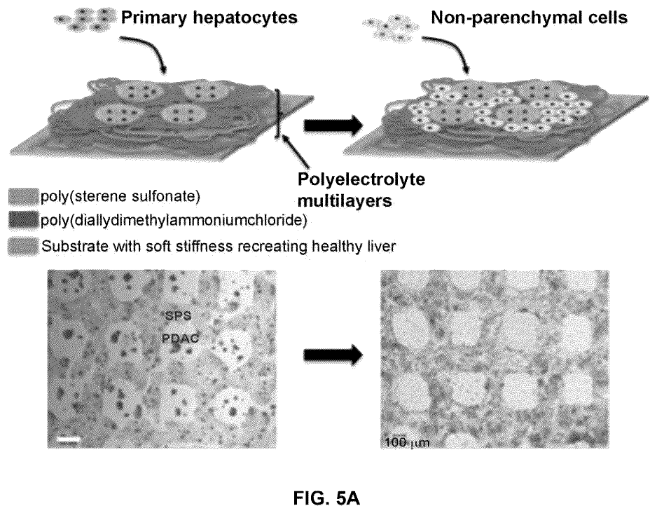

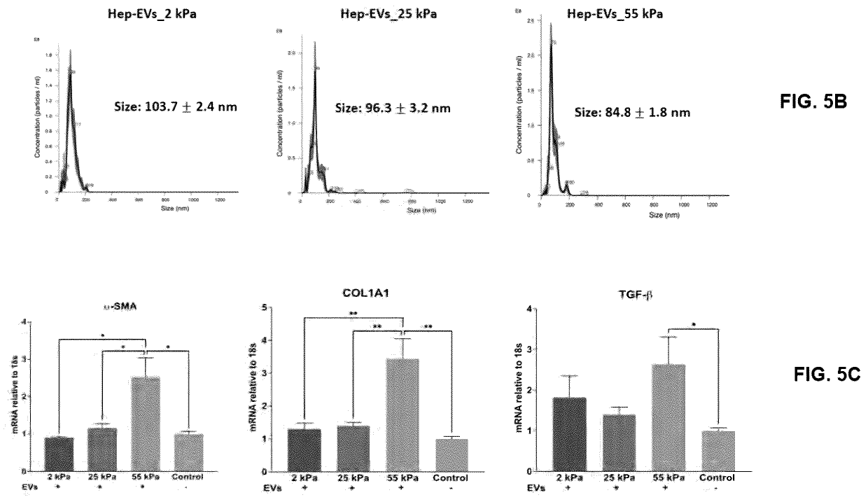

PEM films are used as templates for patterned co-cultures of primary PHHs/HSCs on the BEASTS platform, as illustrated in FIG. 5A. Unlike a PHH overlay with Matrigel, this novel approach allows for co-culturing various cells in a patterned manner, resembling the co-localization of these cells in the liver. To create patterned co-cultures on PEMs, the preferential attachment and spreading of PHHs on one surface over the other will be capitalized to obtain patterned co-cultures of nonparenchymal liver cells and hepatocytes on synthetic PEM surfaces. Such patterned co-cultures preserve hepato-specific function much longer than a patterned mono-culture of PHHs, thereby maintaining the viability and function of both the non-parenchymal liver cells and PHHs. To separate co-cultured cells for Western blot (WB), PCR, or other analysis, cells will be stained with two dyes (CFDSE and PKH26) before seeding and then sorted by FACS.

For example, a method for making a cell culture system includes subjecting the PDMS surfaces to a Harrick plasma cleaner (Harrick Scientific Corporation, Broading Ossining, NY) for 3 min at 0.15 torr and 50 sccm flow of O₂ in a plasma chamber. Following the plasma treatment, the PDMS surfaces were coated with PEMs using a Carl Zeiss slide stainer to create PEM-coated PDMS. In certain embodiments, PDAC and SPS are strong polyelectrolytes, resulting in smooth, homogeneous and stable PEM films suitable for cellular studies and used for several of the tissue/organ models to coat the PDMS surfaces. Both polymers were prepared with deionized (DI) water at concentrations of 0.02M and 0.01M respectively with the addition of 0.1M NaCl salt. To form the first bilayer, the PDMS substrates were immersed for 20 min in a polycation solution (PDAC). Following two sets of 5 min rinses with agitation in DI water, the PDMS substrates subsequently placed in a polyanion (SPS) solution and allowed to deposit for 20 min. Afterwards, PDMS substrates were rinsed twice for 5 min each in DI water. This process was repeated to build multiple layers. These experiments were performed using 5 bilayers (i.e., 10 monolayers). The average thickness of a PDAC/SPS film on various substrates was characterized to be 3.7-4 nm. About 5 bilayers of PDAC/SPS was sufficient to produce smooth and homogeneous PEM films. Before seeding cells, PDMS substrates were placed into tissue culture plates and exposed to UV overnight to sterilize the surfaces.

### Hepatocytes Derived EVs on Stiffness Impacts Hepatic Cells Functions

The cell-cell communication was examined by observing the indirect cell communication. That is to focus on small information, but significant cargoes packed inside micro-vesicles such as extracellular vesicles. Extracellular vesicles (EVs) are lipid bilayers-bound vesicles consist of various lipids, RNA, DNA, and proteins that parents' cells deliver to their recipient cells as a method of communication during healthy and diseased stages of liver diseases. Here in this study, EVs were isolated from hepatic cells (Hep-EVs) line, HepG2, cultured on 2 kPa, 25 kPa, and 55 kPa. The hepatocytes derived EVs were then incubated with LX-2 for 48 hours prior to observing the LX-2 activation. First, higher concentration of Hep-EVs was found on higher stiffnesses (25 kPa and 55 kPa) compared to the 2 kPa as seen in FIG. 5B. Interestingly, when the Hep-EVs were incubated with LX-2, the hepatic stellate cells biomarkers such as a-SMA, COL1A1, and TGF-β were significantly impacted on all stiffnesses. Hep-EVs isolated from the 55 kPa treated with LX-2 cells were increased the profibrotic biomarkers, such as a-SMA, COL1A1 compared to the 2 kPa as seen in FIG. 5C. This further confirmed that stiffness plays a plausible role not only with a direct cell contact such as those on co-culture but also with an indirect hepatocytes cell contact such as those incubated with the hepatic stellate cells.

PHHs with quiescent HSCs (LX2) are cultured on the BEASTS platform at various stiffnesses (to prevent nonspecific activation of HSCs). Cells are treated with varying concentrations of ethanol (0 or 5, 10, 25, 50, and 100 mM) for up to 30 days. Normal and ethanol-containing media are replenished daily.

To inhibit ethanol metabolism, the cells are co-incubated with 5 mM 4-methyl pyrazole (4-MP) for 24 hours, which inhibits ADH and CYP2E1 activity. To determine the possible contribution of HSCs to ethanol metabolism and to determine if ethanol alone can activate these cells, quiescent HSCs alone are subject to the above-described conditions in the presence and absence of 4-MP. After the hepatocytes-HSCs co-culture and/or HSC monoculture experiments, the incubation media is collected for measurement of ethanol metabolism (metabolites). ALT and LDH are measured as an index of cytotoxicity (necrosis). To separate hepatocytes from HSCs following the co-culture experiment, the cells are trypsinized and subjected to flow cytometry. By WB and RT-PCR analyses, the following parameters are determined. In hepatocytes, inflammatory mediators are measured as previously described. In HSCs, pro-fibrotic signaling via TGFβ (TGF-BRI and TGF-βRII) and SMAD 2, 3, the activated phenotype via a-SMA, and the ECM via COL1A1, COL1A2 and CTGF are measured. Also, pro-inflammatory cytokine/chemokine mRNAs (M-CSF, MCP-1, MIP-2, IL-8, ICAM1, TNFα, and the chemokine CXCL10) are measured by RT-PCR and ELISA. Profibrotic HSC activation is assessed by Col1A1, TGFB, prostaglandin D2 receptor, and tissue inhibitor of metalloproteinases 1 (TIMP1) mRNAs by RT-PCR; a-smooth muscle actin (SMA) and Col1A1 protein expression are measured by WB analysis. LDH is measured as an index of cytotoxicity (necrosis).

### BEASTS Platform for Preeclampsia

Preeclampsia (PE) is a pregnancy-associated complication marked by hypertension and proteinuria, typically arising after 20 weeks of gestation. As a significant factor in maternal mortality and morbidity, PE affects 5-7% of all pregnant women, resulting in over 70,000 maternal and 500,000 fetal deaths each year worldwide. At present, no effective therapeutic methods are available for treating PE, and severe cases require preterm labor induction to impede disease progression, subsequently heightening the risk of chronic illness in neonates.

Clinical studies employing shear-wave elastography have reported a substantial increase in placental tissue stiffness under preeclamptic conditions. An RNA-seq analysis revealed alterations in extracellular matrix (ECM) functions, oxidative stress, and mitochondrial activity in preeclamptic pregnancies. However, the impact of stiffness-driven placental dysfunction and its underlying molecular mechanisms remain underexplored. To address this gap, the BEASTS system was developed with elastic moduli of 8, 25, and 55 kPa, to simulate healthy, preeclamptic, and severe preeclamptic conditions, respectively. Human placental trophoblast cells were cultured on these substrates for in vitro studies. Multimodal gene expression analyses were conducted in clinical samples and BEASTS systems. Significant alterations were observed in stiffness-related markers governing critical cellular functions such as proliferation, apoptosis, adhesion, angiogenesis, and contractility. Changes in cell morphology, proliferation, and migration in response to varying stiffness were evaluated. Cell cultures exhibited increased reactive oxygen species (ROS) production and reduced glutathione synthesis with increasing stiffness. Metabolic assessments using Seahorse technology revealed an upregulation of mitochondrial respiration and a decrease in glycolysis function as stiffness increased. The effect of substrate stiffness on nuclear factor erythroid 2-related factor 2 (Nrf2) activation in HTR8 cells and its influence on relevant gene expressions were also evaluated. The therapeutic potential of sulforaphane in modulating Nrf2 gene behavior to mitigate preeclampsia was also assessed. Stiffness changes significantly impact trophoblast function in preeclampsia. The BEASTS model can be employed for further exploration of signaling pathways involved in disease progression and the development of potential therapeutics.

Trophoblast cells, which constitute the majority of the placenta, are essential for nutrient transport, gas exchange, and waste removal, thereby playing a critical role in both fetal development and maternal health. Although the precise etiology of PE remains elusive, it is associated with placental dysfunction. Shallow trophoblast invasion during early pregnancy and constriction of maternal blood vessels are considered to be linked to PE, leading to increased blood pressure and diminished blood supply to the fetus, placenta, and various maternal organs. Consequently, these factors give rise to elevated ROS levels and metabolic imbalances, potentially contributing to endothelial dysfunction in women with PE during later stages of pregnancy.

Recent non-invasive clinical studies have utilized Shear-wave elastography (SWE) to measure placental stiffness in vivo for healthy and preeclamptic placentas. The results have consistently shown higher placental stiffness in preeclampsia patients (25 kPa) compared to healthy pregnant women (10 kPa), with severe cases reaching up to 70 kPa. The molecular mechanisms underlying the impact of stiffness on placental dysfunction and metabolic alterations during PE remain underexplored.

Numerous studies have demonstrated that signaling pathways associated with various biological functions, such as energy utilization, oxidative stress, cell proliferation, invasion, migration, angiogenesis, and differentiation, are significantly altered in preeclampsia. The placenta predominantly depends on oxygen to generate energy via oxidative phosphorylation, an aspect of mitochondrial respiration. Preeclampsia is associated with an overproduction of ROS and mitochondrial dysfunction. Adaptive mitochondrial responses to ROS contribute to changes in mitochondrial function, which in turn play a critical role in pregnancy complications related to PE.

Mechanical forces are known to influence cell fate during differentiation, especially in early embryo development, where self-organization and germ layer maturation depend on both intrinsic and external mechanical cues. As development advances, mechanical forces from the extracellular matrix guide cell fate during organogenesis and specialization of fetal organs. Yes-associated protein (YAP), a transcription coactivator in the Hippo pathway, is an essential mechano-signaling pathway influenced by mechanical cues like stiffness. Recent clinical studies have revealed that reduced YAP levels contribute to trophoblast dysfunction, impacting trophoblast proliferation, apoptosis, and invasion. These findings are consistent with observations of altered Hippo pathway genes in the placentas of patients with severe preeclampsia.

Analogous functional changes were observed in stiffness-based in vitro models, where stiffness hindered liver function, triggered metabolic dysfunction, and modified mitochondrial respiration in hepatocytes. Intriguingly, these biochemical alterations in the liver model bear a close resemblance to the clinical findings seen in the placentas of PF patients, though the exact pathological causes remain unknown. The available evidence collectively points to a relationship between alterations in stiffness and the progression of preeclampsia. Despite this, there has been limited research on the effects of stiffness on placental dysfunction and the associated molecular mechanisms. Effectively managing preeclampsia necessitates a more comprehensive understanding of the pathogenesis of stiffness-induced placental signaling and the identification of novel therapeutic targets. This knowledge gap may result from difficulties in creating in vivo animal models for PE, engineering surfaces with adjustable elasticity for in vitro culture, culturing and maintaining placenta cells on synthetic protein-free scaffolds, and the scarcity of studies exploring the mechanisms connecting biomechanical signaling in the placenta during PE with placental metabolism.

The impact of substrate stiffness on metabolic dysfunction and alterations in trophoblast function, which may lead to complications during PE, were investigated using the BEASTS platform. To establish a connection between molecular mechanisms in PE and healthy conditions, RNA sequencing data was examined, differentially expressed genes (DEGs) were identified, and enrichment analysis was conducted to pinpoint key cellular processes associated with PE. The BEASTS platform facilitated the examination of the relationship between these clinical findings and their association with substrate stiffness. This platform employs PDMS substrates with polyelectrolyte multilayer film coating technology to create mechanically tunable surfaces that mimic physiological and pathological stiffness conditions during preeclampsia. BEASTS platforms were developed with substrates with stiffness values of 8, 25, and 55 kPa, corresponding to healthy, preeclamptic, and severe preeclamptic conditions, respectively. In the in vitro experiments, the human HTR8 cell line of transfected trophoblasts was utilized, which serves as a well-established model for investigating developmental changes and functions in the placenta. The cells were cultured on the BEAST platform in multiple replicates. Extensive gene expression analyses were performed in clinical samples and BEASTS systems to observe changes in stiffness-related markers governing critical cellular functions. Furthermore, changes in cell morphology, proliferation, and migration were investigated in response to varying stiffness. Mitochondrial energy metabolism, the effect on cellular oxidative stress, and the Nrf2 pathway were examined following the change in stiffness. Additionally, the therapeutic potential of sulforaphane in modulating Nrf2 gene behavior to mitigate preeclampsia was assessed.

Analysis of healthy and preeclamptic tissue samples revealed altered cellular mechanisms and differentially expressed genes, including GPR18.

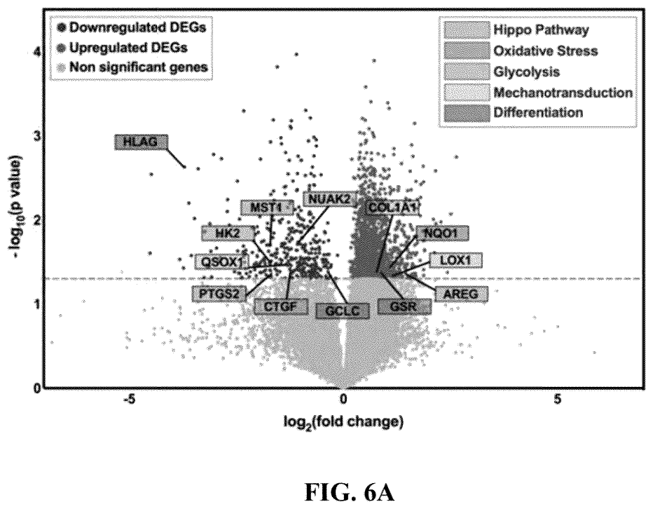

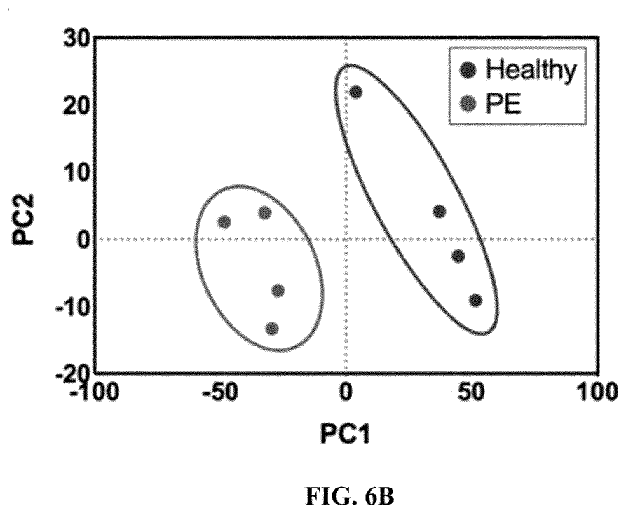

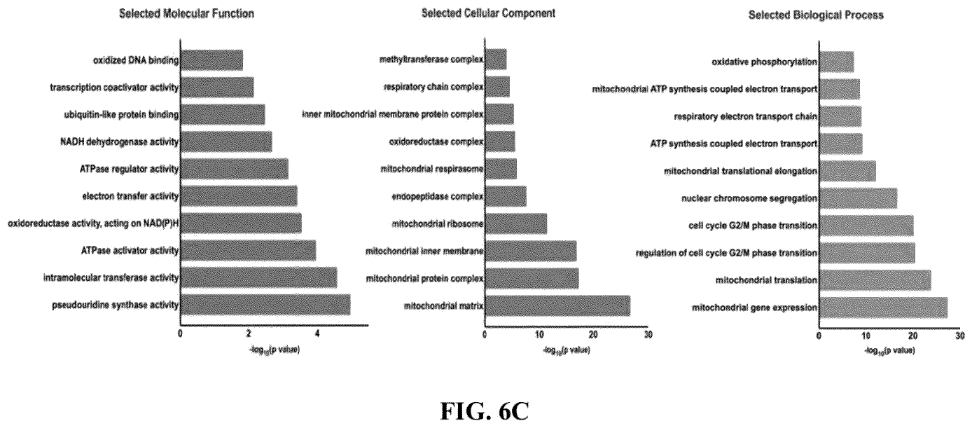

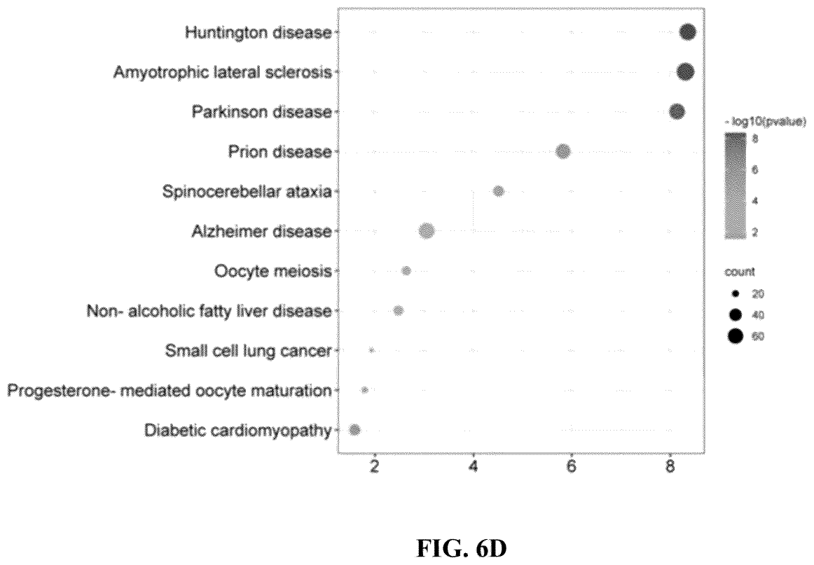

The RNA sequencing data was procured from UNMC (Dr. Berry's lab) using tissue samples from both healthy and preeclamptic women, with four samples in each group. Upon processing the data, 1,843 DEGs were identified including 1,465 significantly upregulated genes and 378 significantly downregulated genes. A volcano plot illustrates the DEGs between the two experimental conditions, with genes color-coded based on their adjusted p-value and log fold change. Red dots signify significantly upregulated genes, while blue dots indicate significantly downregulated genes (FIG. 6A). This volcano plot emphasizes the marked genes that have a critical impact on placental development. Additionally, the PCA plot of DEGs demonstrates the distinct clustering of healthy and preeclamptic samples according to their gene expression profiles (FIG. 6B).

The GO KEGG enrichment approach was utilized to identify the specific molecular functions, cellular components, and biological processes that were significantly enriched in placental tissues from healthy and preeclamptic pregnancies (FIG. 6C). These enriched molecular functions encompassed a range of activities, including oxidoreductase, electron transfer, pseudouridine synthesis, ATPase activation and regulation, and transcription coactivation, which suggest their involvement in energy metabolism, RNA modification, and gene expression regulation. The cellular components identified are all related to mitochondria and play crucial roles in metabolic reactions, protein synthesis, electron transport, and mitochondrial protein function and membrane structure. The enriched biological processes identified here, such as mitochondrial gene expression, regulation of the cell cycle G2/M phase transition, and oxidative phosphorylation, are essential for maintaining proper mitochondrial function and energy production, which is crucial for a healthy pregnancy. The dysregulation of these processes in preeclampsia emphasizes the significance of mitochondrial function and oxidative phosphorylation in managing complications associated with preeclampsia. The enrichment analysis here demonstrates the clear correlation between mitochondrial function, oxidative phosphorylation, ECM components, and their association with altered energy metabolism and oxidative stress in preeclampsia. The disease enrichment analysis identified oocyte meiosis and progesterone-mediated oocyte maturation as important pathways connected to placental health and preeclampsia (FIG. 6D). Both oocyte meiosis and progesterone-mediated maturation play vital roles in egg formation, fertilization, and embryo development. Gaining insights into the underlying mechanisms of these reproductive pathways associated with placental health and preeclampsia is essential for addressing related complications.

GPR18 has been investigated for its involvement in inflammation and oxidative stress in various diseases, including preeclampsia. It is known to regulate trophoblast cells and has been implicated in numerous other physiological processes such as intraocular pressure regulation, neuroimmunomodulation, arterial blood pressure regulation, and metabolic disorders. In clinical samples, a comparison of GPR18 expression between healthy and preeclamptic conditions revealed a significantly higher percentage of preeclamptic patients expressing GPR18 (FIG. 6E). Immunohistochemistry analysis was performed to visualize GPR18 protein expression in clinical samples from healthy and preeclamptic pregnancies (FIG. 6F). Placental tissues were stained with DAB, and the staining intensity was quantified. The results demonstrated a significant increase in DAB intensity in preeclamptic placental tissues compared to healthy tissues (FIG. 6G). The upregulation of GPR18 gene and protein expression, which is linked to inflammation and oxidative stress, may contribute to the pathogenesis of preeclampsia, as these factors are key features of the condition.

RNA sequencing data analysis was used to identify DEGs in placental tissues from healthy and preeclamptic pregnancies. Illustrated by a volcano plot and principal component analysis (PCA) plot, the analysis highlights the differences in gene expression between the two conditions and emphasizes the impact of specific genes on placental development. The GO KEGG enrichment analysis revealed significant enrichment of molecular functions, cellular components, and biological processes in placental tissues, highlighting the importance of mitochondrial function, oxidative phosphorylation, and energy metabolism in maintaining a healthy pregnancy and their dysregulation in preeclampsia. Disease enrichment analysis identified oocyte meiosis and progesterone-mediated oocyte maturation as crucial pathways related to placental health and preeclampsia. Furthermore, there was an upregulation of GPR18, a gene associated with inflammation and oxidative stress, in preeclamptic patients. This upregulation indicates that GPR18 may contribute to the pathogenesis of preeclampsia, as inflammation and oxidative stress are key features of the condition. By utilizing the BEASTS platform, one can identify key parameters for developing effective therapeutic interventions to address preeclampsia-related complications.

Molecular analysis utilizing BEASTS platform revealed altered cellular functions correlating with clinical data

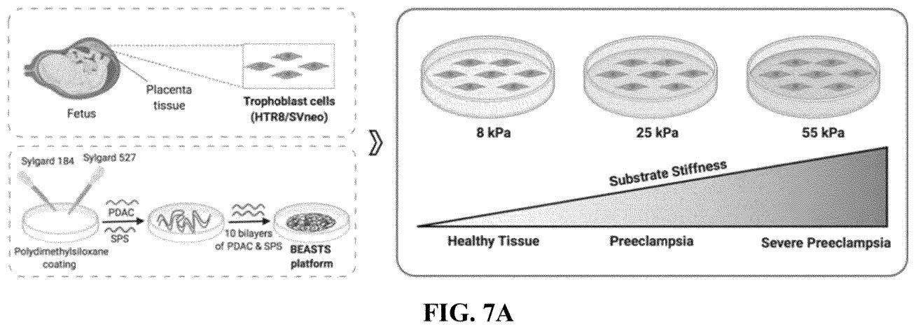

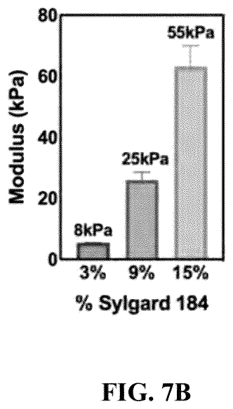

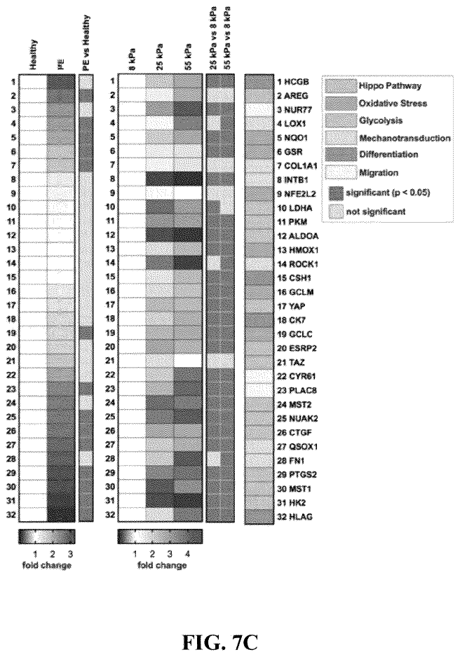

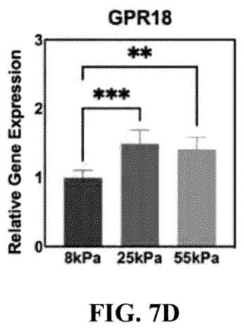

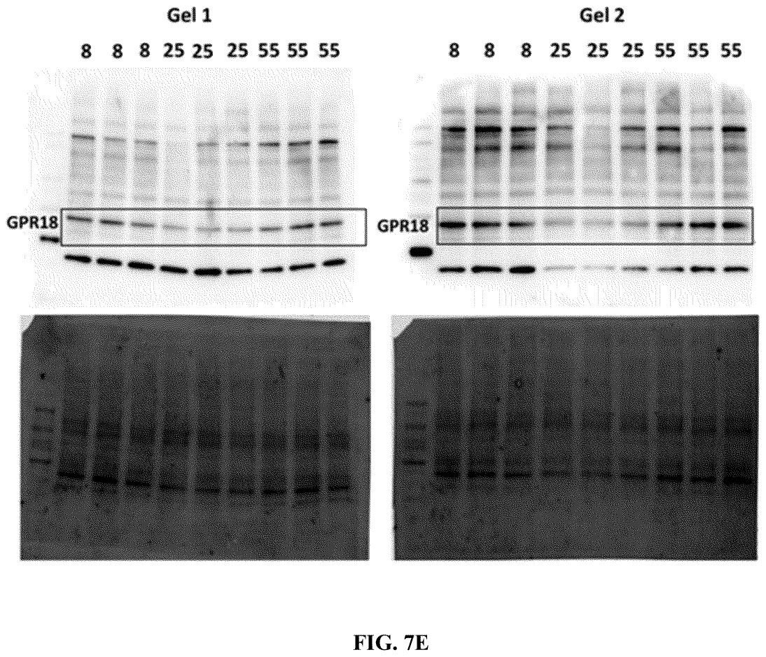

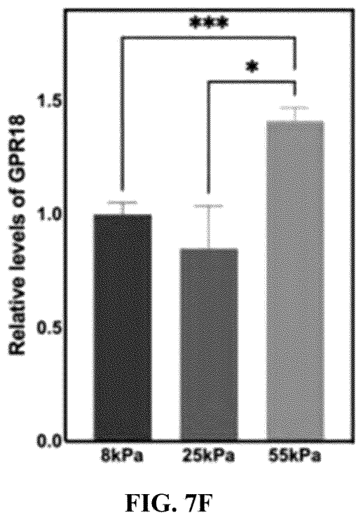

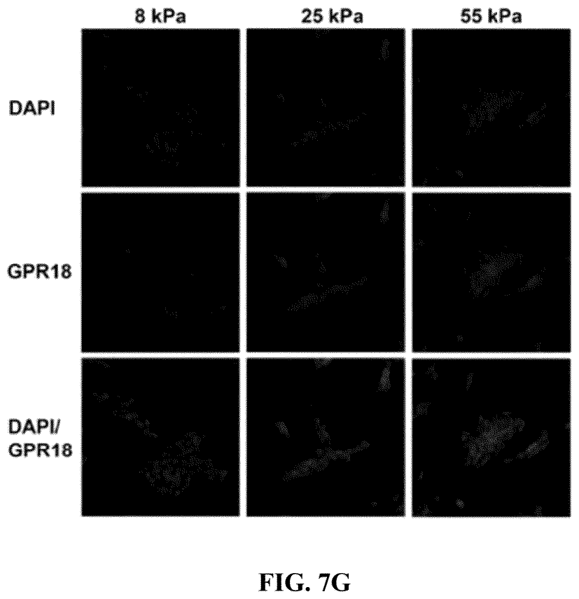

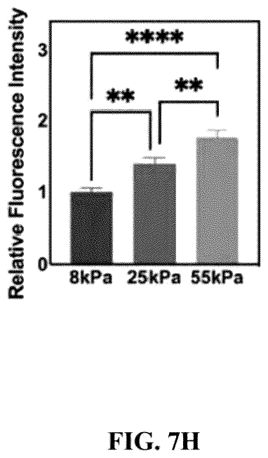

To investigate the molecular mechanisms involved in the mechanobiology of the placental microenvironment, a 2D in vitro BEASTS platform was developed to represent specific stages in the progression of preeclampsia (FIG. 7A). The BEASTS platform was fabricated using commercially available PDMS polymers, Sylgard 184 and Sylgard 527, which were combined in defined mass ratios to achieve physiologically relevant elastic moduli. This allowed for the creation of biomaterials with tunable elastic moduli across three orders of magnitude. Sylgard 184 and Sylgard 527 were mixed in mass ratios of 3:97, 9:91, and 15:85 to model distinct stages of preeclampsia, with stiffnesses of 8 kPa representing a healthy condition, and 25 kPa and 55 kPa representing preeclampsia and severe preeclampsia conditions, respectively. These substrates were coated with 10 bilayers of Polydiallyldimethylammonium chloride (PDAC) and Sulphonated polystyrene (SPS) polymers using layer-by-layer assembly to form a smooth and homogeneous film. PDAC was selected as the polycation based on its high positive charge density and biocompatibility, and cell adhesion properties, while SPS was chosen for its high negative charge density and widespread use in PEM assembly. The HTR8/SVneo trophoblast cell line, derived from human first-trimester placental tissue, was used to study trophoblast cell function in healthy and PE conditions using the BEASTS platform. Cells were seeded at 25% confluency and cultured for four days. The stiffness of the BEASTS platforms was evaluated by measuring their Young's modulus with a TMSPro texture analyzer (FIG. 7B). The values obtained were consistent with clinical findings: 8.42±0.18 kPa (healthy), 25.96±4.5 kPa (PE), and 56.6±5.1 kPa (severe PE).

Gene expressions related to cellular functions identified from RNA sequencing in FIGS. 6A-6G, including migration, Hippo pathway, oxidative stress, glycolysis, mechanotransduction, and differentiation (FIG. 7C), were compared between clinical data and the BEASTS model to establish a stiffness-based preeclampsia model. Cells were cultured on the BEASTS platform at 8 kPa, 25 kPa, and 55 kPa to mimic healthy and preeclampsia conditions, and RNA was extracted for PCR analysis of gene expression changes. The heatmap shows differences in gene expression between healthy and preeclampsia conditions in clinical samples and the BEASTS platform at 8 kPa, 25 kPa, and 55 kPa.

Several genes associated with the Hippo signaling pathway were evaluated, which regulate vital cellular processes such as cell proliferation, apoptosis, differentiation, and tissue growth. AREG stimulates cell proliferation and migration, YAP and TA7. function as transcriptional coactivators involved in cell growth and tissue repair, ESRP2 regulates alternative splicing and plays a role in epithelial-mesenchymal transition, MST1 and MST2 inhibit cell proliferation and promote apoptosis, NUAK2 is involved in cell adhesion, migration, and energy metabolism, CTGF is involved in cell proliferation, migration, adhesion, and extracellular matrix remodeling, and PTGS2 synthesizes prostaglandins involved in inflammation, pain, and fever. Collectively, these Hippo pathway genes are critical for maintaining cellular and tissue homeostasis, and their dysregulation has been linked to various pathological conditions. Most of the gene expressions were downregulated in both clinical data and the BEASTS platform, with statistical significance noted between healthy and PE conditions in clinical data and across different stiffnesses mimicking healthy and PE conditions in the BEASTS platform. This data indicates that crucial functions such as cell proliferation, adhesion, differentiation, and migration are altered in placental cells, leading to preeclamptic conditions.

Several key genes associated with oxidative stress in preeclampsia were examined, including NQO1, GSR, HMOX1, GCLM, and GCLC. NQO1 and GSR reduce reactive oxygen species and detoxify harmful substances, while HMOX1 generates antioxidant molecules and has cytoprotective and anti-inflammatory effects. GCLM and GCLC synthesize glutathione, a vital cellular antioxidant. In the BEASTS platform, these genes were significantly downregulated in 25 kPa and 55 kPa preeclampsia conditions compared to 8 kPa healthy conditions. The clinical studies and BEASTS platform showed similar changes in GCLC, GCLM, and NFE2L2 gene expression, indicating a correlation between clinical and BEASTS platform in relation to oxidative stress conditions during preeclampsia.

Key glycolysis-related genes were analyzed in the BEASTS model to investigate the critical metabolic pathway of glycolysis, which breaks down glucose to produce ATP and regulate cellular metabolism. HK2 catalyzes the first step, LDHA converts pyruvate to lactate during anaerobic conditions, PKV converts phosphoenolpyruvate to pyruvate, and ALDOA facilitates the reversible conversion of fructose-1,6-bisphosphate. The genes related to glycolysis were significantly downregulated in preeclampsia conditions (25 kPa and 55 kPa) compared to healthy conditions (8 kPa). HK2 was also downregulated in clinical data in preeclampsia compared to healthy conditions. Altered glycolysis genes are associated with various pathologies. Studying these genes in the context of placental stiffening can reveal metabolic changes in preeclampsia and inform potential treatments.

Key genes were evaluated, such as those involved in mechanotransduction, crucial for cellular processes like proliferation, differentiation, migration, and ECM remodeling, and necessary for proper cellular function and response to mechanical stimuli. LOX1 cross-links collagen and elastin fibers to stabilize ECM, while COL1A1 contributes to tissue strength. INTB1 is a cell receptor involved in cell-ECM adhesion and signaling. ROCK1 regulates cell contractility and mechanical responses. QSOX1 affects ECM stability by catalyzing disulfide bond formation, and FNI is a glycoprotein involved in cell adhesion, migration, and ECM organization. In the BEASTS platform, LOX1, INTB1, ROCK1, QSOX1, and FN1 genes were significantly changed in preeclamptic conditions at 25 kPa and 55 kPa compared to the healthy condition at 8 kPa. Clinical data also showed similar changes in LOX1, COL1A1, QSOX1, and FN1 genes between healthy and preeclamptic conditions, consistent with the findings in the BEASTS platform. By understanding the role of mechanotransduction in preeclampsia, insights into the underlying molecular mechanisms can be gained and potential therapeutic strategies can be developed for addressing altered cellular function and response to mechanical forces.

Key differentiation-associated genes were analyzed, such as those genes that regulate placental development, trophoblast differentiation, and immune regulation during pregnancy. HCGB and CSH1 are placental hormones essential for maintaining progesterone production and modulating maternal metabolism for fetal growth. CK7 is an intermediate filament protein expressed in trophoblasts, indicating trophoblast differentiation. HLAG, expressed by extravillous trophoblasts, has immunomodulatory functions that protect the fetus from maternal immune attack. All these genes were significantly upregulated in 25 kPa and 55 kPa preeclamptic conditions in the BEASTS model compared to the healthy 8 kPa condition. Additionally, HCGB and HLAG were also altered in clinical data between healthy and preeclamptic conditions, with HCGB showing similar changes in both the clinical and BEASTS model. Together, these differentiation-associated genes contribute to placental development, trophoblast differentiation, and immune regulation during pregnancy. Dysregulation of these genes may lead to pregnancy complications, such as preeclampsia; studying their role in healthy and preeclamptic conditions provides valuable insights into underlying molecular mechanisms.

Cell migration is crucial in physiological and pathological events, including placental development. Genes involved in trophoblast migration and overall placental health were evaluated. NUR77 is a nuclear receptor that regulates target genes involved in cell proliferation, differentiation, and migration, playing a role in trophoblast migration and invasion during placental development, while CYR61 promotes trophoblast migration and invasion and PLAC8 regulates trophoblast migration and invasion, all of which are essential for proper placental development and successful pregnancy. The expression levels of all these genes were significantly higher in the 25 kPa and 55 kPa preeclamptic conditions in the BEASTS platform compared to the healthy 8 kPa condition. Additionally, the clinical data showed altered expression of PLAC8 between the healthy and preeclamptic conditions. These genes contribute to placental development by regulating trophoblast migration and invasion, and their dysregulation may cause pregnancy complications. Studying their role in both healthy and preeclamptic conditions can provide insights into placental development mechanisms.

The GPR18 gene, known for its involvement in inflammation and oxidative stress in preeclampsia, displayed upregulation in the clinical data (FIGS. 6E, 6F, and 6G). To further investigate this connection, PCR analysis was performed to assess GPR18 gene expression in the BEASTS platform (FIG. 7D). The results revealed a significant increase in GPR18 expression with escalating stiffness, particularly in preeclampsia conditions. Western Blot analysis confirmed GPR18 protein expression in BEASTS, with markedly higher levels at 55 kPa compared to 8 kPa and 25 kPa (FIGS. 7E and 7F). Additionally, immunostaining analysis and quantification of GPR18 localization in BEASTS demonstrated that GPR18 levels notably rise in tandem with increasing stiffness (FIGS. 7G and 7H). These findings from the BEASTS platform align with the clinical data on gene expression and immunohistochemistry (IHC) analysis in preeclamptic women, as illustrated in FIGS. 6E, 6F, and 6G.

The molecular analysis conducted using the BEASTS platform closely resembles the clinical data obtained from preeclamptic women, establishing a clear connection between mechanical properties, specifically substrate stiffness, and alterations in molecular mechanisms during pregnancy complications such as preeclampsia. This alignment of the BEASTS platform emphasizes the potential of the BEASTS platform as a valuable tool for investigating trophoblast behavior, energy metabolism, and responses to oxidative stress under both healthy and preeclamptic conditions. By utilizing this model, one can gain a deeper understanding of the complex interplay between mechanical properties and molecular processes in the placenta, which could potentially contribute to the development of novel therapeutic strategies for managing preeclampsia and related complications.

Substrate stiffness alters HTR8/SVneo morphology and enhances cell proliferation and

migration in a stiffness-dependent manner in the BEASTS platform

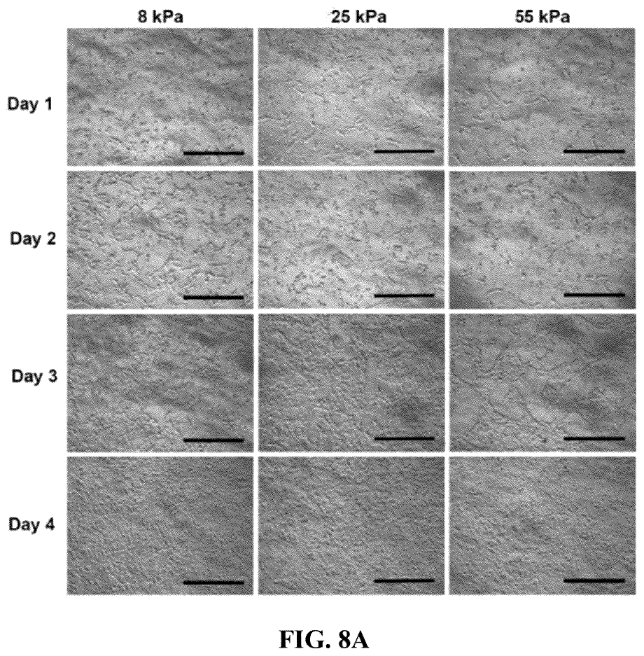

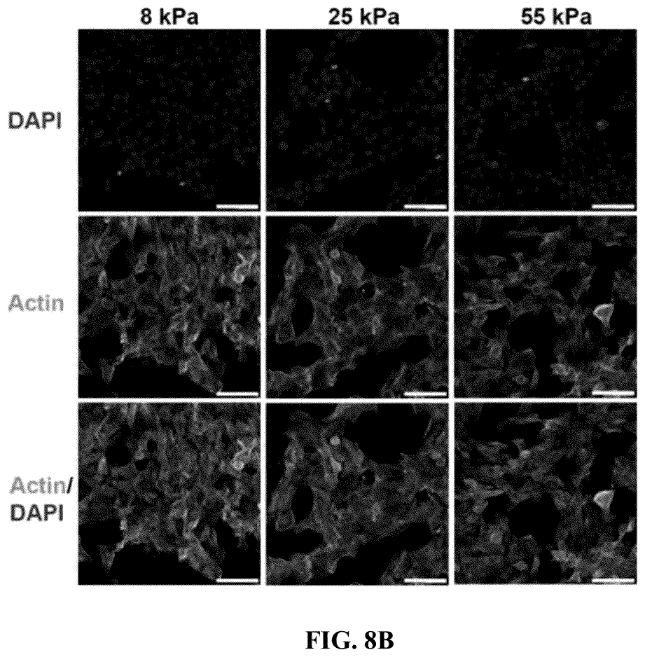

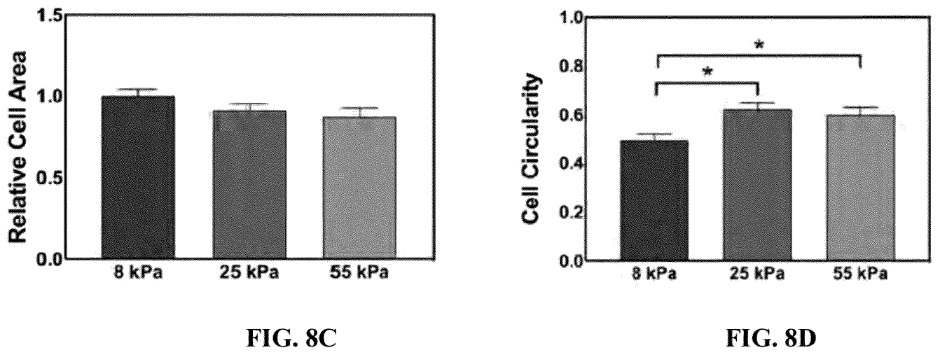

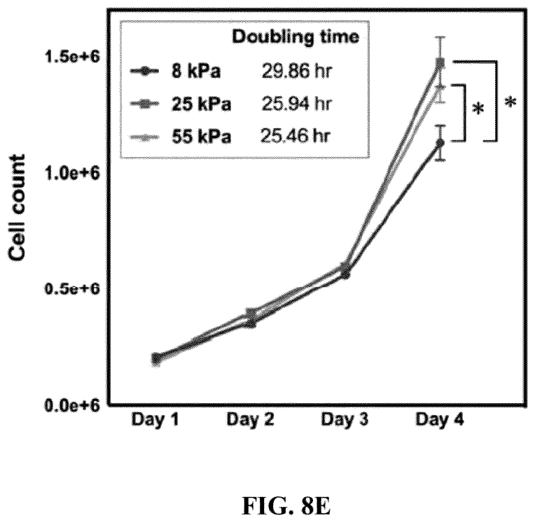

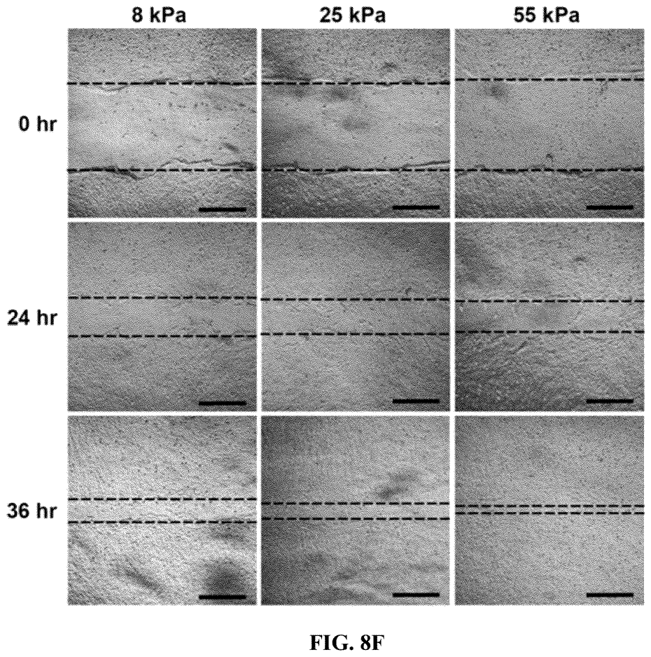

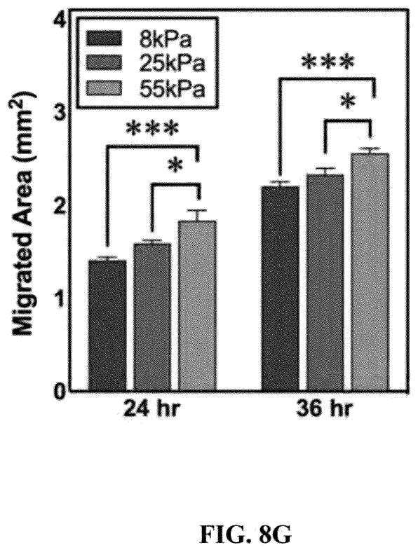

Representative phase contrast images were taken over four consecutive days to observe the effects of substrate stiffness on HTR8 cell morphology (FIG. 8A). Cells were cultured on three different stiffness levels for four days, allowing for the examination of their morphology and attachment properties. On standard tissue culture polystyrene (TCPS) plates, cells adopted an elongated shape after attachment, whereas on the stiffness gels, cells exhibited a more rounded morphology. Within approximately 48 hours, these cells on the gels transitioned into a more elongated shape. Cell attachment was reduced on higher stiffness substrates compared to lower stiffness ones. However, no other significant morphological changes were visible in response to varying stiffness levels.

The impact of substrate stiffness on cell morphology was evaluated by immunostaining the actin filament network within the cell cytoskeleton, which is responsible for providing mechanical support and determining cell shape (FIG. 8B). Cells were cultured on stiffness plates for four days and then fixed using paraformaldehyde. They were subsequently incubated with an actin dye (green) and DAPI (nucleus, blue) for 20 minutes before images were captured using a confocal microscope. The cell area and circularity were quantified using ImageJ software. The relative cell area was slightly smaller for cells on higher stiffness substrates compared to 8 kPa (healthy), but the difference was not statistically significant (FIG. 8C).

Cell circularity is a unitless parameter that measures the roundness of cells, with values ranging from 0 to 1, where 1 represents a perfect circle. Cells on the 8 kPa surface had a circularity value of 0.49, while cells on stiffer surfaces had values of 0.62 for 25 kPa and 0.59 for 55 kPa, respectively (FIG. 8D). HTR8 cells on stiffer surfaces had a more rounded morphology, with a significant increase in circularity as stiffness increased. This observation is consistent with the cells' typical elongated shape on TCPS under healthy conditions and their transition to a more rounded appearance under unhealthy or strained conditions. These measurements further corroborate the general observations made regarding cell morphology and stiffness.

Proliferation studies were conducted by culturing cells on substrates with stiffnesses representative of healthy and diseased states for four days (FIG. 8E). Cells were seeded at 25% confluence and detached at specified time points. Cell counts were obtained using a hemocytometer to determine the doubling time for each stiffness condition. For the first three days, there were no significant differences in cell numbers across the varying stiffnesses. However, on day four, the cell counts were significantly higher for the stiffer substrates (25 kPa and 55 kPa) representing preeclampsia, compared to the 8 kPa healthy condition. The doubling rate of cells for 8 kPa was 29.86 hours, while it was lower for higher stiffnesses with 25.94 hours for 25 kPa and 25.46 hours for 55 kPa. Cell proliferation increases with higher substrate stiffness, consistent with the observed increase in proliferation during preeclampsia.

Preeclampsia is known to be associated with impaired trophoblast invasion, which led us to conduct a cell migration assay (FIG. 8F). HTR8 cells were cultured on BEASTS surfaces with stiffnesses of 8 kPa, 25 kPa, and 55 kPa for four days until they reached confluence. A scratch was then made in the cell layer using a pipette tip, and phase contrast images were taken at 24-hour and 36-hour time points to monitor the area migrated by the cells until the wound was nearly completely covered. At both time points, cells cultured on stiffer substrates (25 kPa and 55 kPa) migrated significantly more than those on the 8 kPa substrate, representing healthy conditions. Quantification of the migrated area using ImageJ software showed a significant increase in migration with increasing stiffness from 8 kPa to 25 kPa and from 25 kPa to 55 kPa (FIG. 8G). By the 36-hour time point, the wound in the 55 kPa condition was completely closed. These findings demonstrate that cell migration is significantly affected by substrate stiffness, with increased migration observed under PE-like conditions. In line with these results, a significant upregulation of migration-associated genes PLAC8, CYR61, and NUR77 was also observed (FIG. 7C) with increasing stiffness in the BEASTS platform. The findings from the migration assay are consistent with the gene analysis data, further strengthening the relationship between substrate stiffness and cell migration in the context of preeclampsia.

The effects of substrate stiffness on HTR8 cell morphology and behavior were evaluated using different stiffness levels representative of healthy and preeclamptic conditions. Cell attachment was observed to be reduced on higher stiffness substrates, and cells displayed a more rounded morphology initially, transitioning to a more elongated shape over time. Actin filament network immunostaining revealed that cells on higher stiffness surfaces exhibited a more rounded morphology with increased circularity compared to those on lower stiffness surfaces. Proliferation studies showed that cell counts were significantly higher for stiffer substrates, representing preeclampsia, and that the cell doubling rate decreased with increasing stiffness. The cell migration assay demonstrated that cells cultured on stiffer substrates migrated significantly more than those on lower stiffness substrates, which is consistent with the upregulation of migration-associated genes observed in the study. Substrate stiffness has a considerable impact on cell morphology, proliferation, and migration, providing valuable insights into the role of mechanical factors in the context of preeclampsia.

Elevated stiffness intensifies oxygen consumption rate and downregulates glycolytic rate in HTR8/SVneo cells

During a healthy pregnancy, mitochondrial function and oxidative phosphorylation are vital for energy production. The dysregulation of these processes in preeclampsia emphasizes their importance in managing complications associated with this condition. Potential changes were evaluated in mitochondrial bioenergetic properties between healthy and preeclamptic pregnancies due to varying placental stiffness, using the BEASTS platform. A Seahorse XFe96 Flux Analyzer was utilized to measure mitochondrial OCR, which records O₂ concentrations in real-time through a fluorescence microplate assay format. Cellular respiration is connected to the electron transport chain, where complexes I-IV harness energy from electron transport to pump protons across the inner mitochondrial membrane. The resulting proton gradient is utilized by complex V to generate ATP. As depicted in the graph, OCR is measured under various conditions. The first three points represent baseline or basal respiration without the addition of inhibitors or drugs. Oligomycin is then introduced, inhibiting ATP synthase at complex V. As oxygen consumption is linked to ATP synthesis, a decrease in OCR is observed, indicating O₂ consumption for ATP generation. Additionally, the OCR due to proton leak can be quantified. Oxygen consumption also takes place at complex IV. When carbonyl cyanide-p-trifluoromethoxyphenylhydrazone (FCCP) is added, it uncouples ATP synthesis from oxygen consumption, leading to the collapse of the proton gradient and disruption of the mitochondrial membrane potential. Consequently, electron flow through the electron transport chain (ETC) becomes uninhibited, and oxygen consumption by complex IV reaches its maximum. Finally, mitochondrial respiration is halted using rotenone, a complex I inhibitor, with some residual oxygen consumption being independent of electron transport chain activity. Mitochondria expel protons to create a proton motive force, which drives protons back through the ATP synthase to produce ATP. However, some protons leak back across the membrane, reducing coupling efficiency. Variations in the kinetics of oxidative phosphorylation components can arise due to factors such as genetic makeup, signaling pathways, oxidative stress, disease, or exposure to pharmacological or toxic compounds. These variations can influence steady-state rates, coupling efficiencies, and energy demand responses.

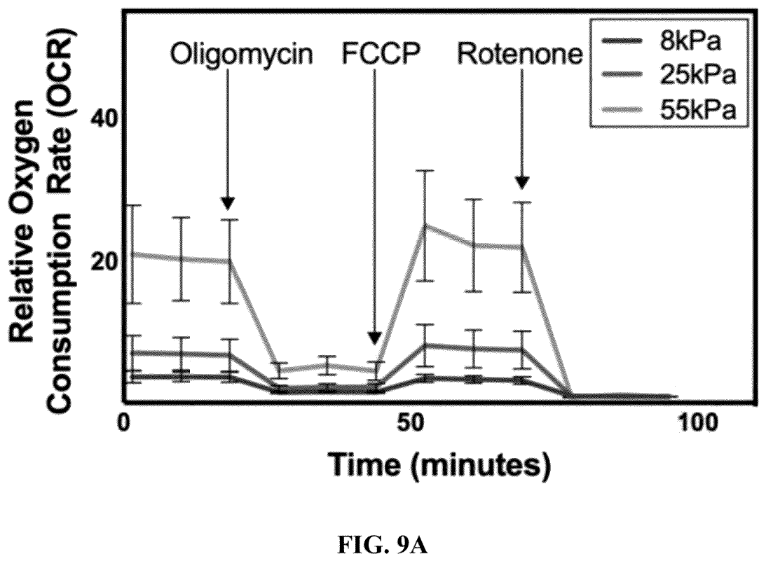

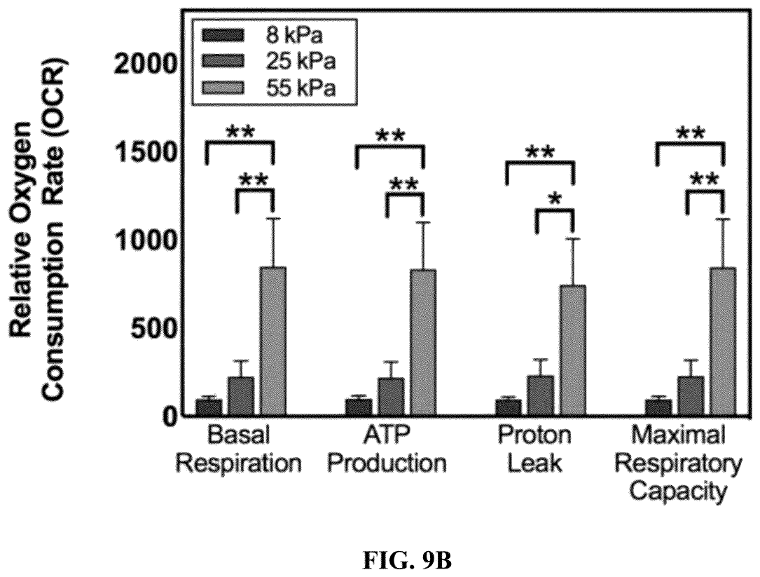

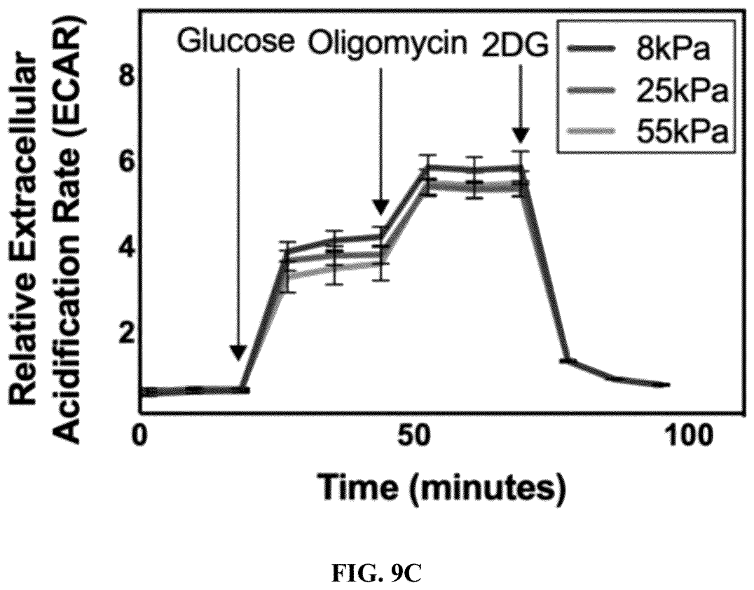

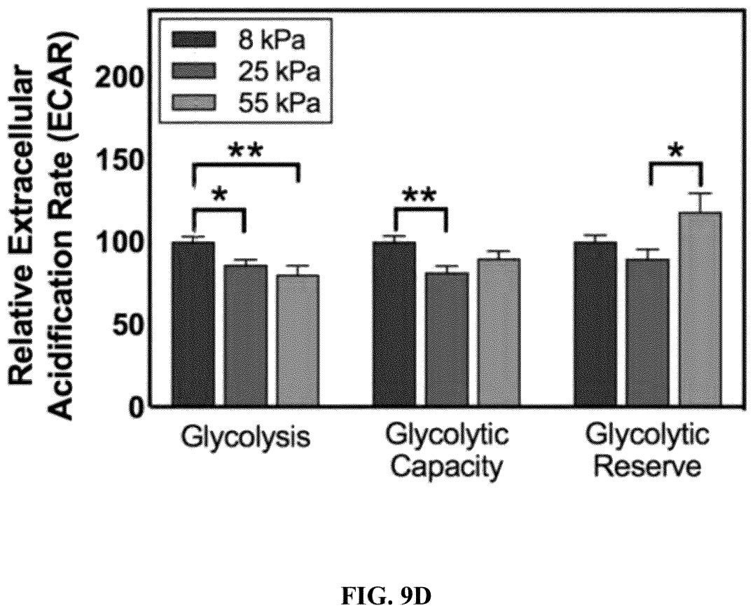

In the experiments using HTR8 cells cultured on three different stiffness levels in the BEASTS platform, a significant increase was observed in mitochondrial respiration as substrate stiffness increased (FIG. 9A). Graphs representing various OCR parameters, such as basal respiration, ATP production, proton leak, and maximal respiratory capacity, demonstrated a significant upregulation in response to increased substrate stiffness (FIG. 9B). These findings underscore the impact of mechanical properties, such as stiffness, on cellular bioenergetics. The differences in the kinetics of oxidative phosphorylation components may be attributed to changes in gene expression and alterations in signaling pathways related to mitochondrial function. For instance, the upregulation of OCR parameters with increasing stiffness could be associated with higher metabolic demand in cells cultured on stiffer substrates, necessitating increased mitochondrial activity to meet these energy requirements. Furthermore, the altered kinetics might suggest a shift in cellular metabolism or a change in the efficiency of mitochondrial complexes in cells grown on stiffer substrates. Cells may adapt their bioenergetic profile to the altered mechanical environment by modulating gene expression, post-translational modifications, or the activity of key enzymes involved in mitochondrial respiration. These changes in mitochondrial function could also be connected to the activation of specific signaling pathways sensitive to mechanical stimuli, such as mechanotransduction pathways. These pathways can convey mechanical signals from the extracellular environment to intracellular signaling cascades, ultimately affecting gene expression, protein function, and cellular metabolism.

In the context of preeclampsia, the observed alterations in mitochondrial bioenergetics might contribute to the development and progression of the disease by exacerbating oxidative stress, impairing trophoblast function, or altering the balance between cell proliferation and apoptosis. Under conditions of maximum energy demand, the mitochondria of preeclamptic women may be less efficient in ATP production compared to those from healthy pregnancies. Moreover, the proportion of oxygen consumption uncoupled from ATP synthesis may be higher in the total oxygen consumed. An elevated proton leak implies reduced coupling efficiency, leading to changes in energy demand response and mitochondrial dysfunction. Mitochondrial respiration regulates apoptosis, cell proliferation, and ROS production, and is continuously generating superoxide as a byproduct. The interconnections between mitochondrial function, oxidative stress, and placental stiffness in preeclampsia can be targeted for further evaluation to uncover potential mechanisms for therapeutic interventions and a comprehensive understanding of the disease.

To investigate glycolysis, a vital component of cellular function, a Seahorse XFe96 Flux Analyzer was employed to measure the ECAR in HTR8 cell cultures within the BEASTS platform. The glycolysis stress test involves measuring the ECAR of previously starved cells. At this basal minimum, ECAR is referred to as non-glycolytic acidification. Subsequently, glucose is introduced to initiate glycolysis, leading to an ECAR increase due to lactate formation. This rise represents the normal glycolysis rate. Next, oligomycin is injected, hindering ATP production, and prompting cells to maximize glycolysis, which results in a secondary ECAR increase. The test concludes by inhibiting glycolysis using the glucose analog 2-DG, returning ECAR to its non-glycolytic level.

The glycolysis data revealed a significant decrease in glycolysis and glycolytic capacity at 25 and 55 kPa. As ECAR primarily originates from glycolytic lactic acid production and carbonic acid formation due to carbon dioxide generated by the tricarboxylic acid (TCA) cycle, the ECAR decrease indicates a reduced glycolysis rate. Preeclamptic women typically display higher basal respiration and increased TCA cycle flux. An increase in glycolytic reserve at 55 kPa was observed compared to 8 and 25 kPa, suggesting a potential deviation from normal function. Reduction in glycolysis can have several implications. Reduced glycolysis may lead to insufficient energy generation, which can impair various cellular processes and functions that rely on ATP. A decrease in glycolysis and glycolytic capacity could signal a shift in cellular metabolism towards alternative pathways, such as oxidative phosphorylation, to produce ATP. This shift may cause cells to rely more heavily on mitochondrial function, which can result in changes to cellular metabolism. Reduced glycolysis may lead to an increase in reactive oxygen species (ROS) production, as cells may become more dependent on mitochondrial oxidative phosphorylation. Elevated ROS levels can cause oxidative stress, potentially damaging cellular components and contributing to cellular dysfunction. Decreased glycolytic activity can influence various cellular functions, such as cell proliferation, differentiation, and survival. In some cases, this can lead to pathological conditions or exacerbate existing diseases. As preeclampsia is associated with elevated ROS levels, this deviation could be influenced by ROS. Increased glycolytic reserve means cells have more capacity to upregulate glycolysis in response to energy demands or stress. It refers to the difference between maximum glycolytic rate and normal rate. It can be beneficial during stress, injury, or other demanding situations. However, an altered glycolytic reserve can also suggest deviations from normal cellular function and pathological conditions. For instance, in preeclampsia, an increase in glycolytic reserve may indicate metabolic imbalances or dysfunctions, such as mitochondrial impairment or increased oxidative stress. Certain studies propose that ROS can inhibit multiple glycolytic enzymes, potentially contributing to this deviation. Reduced glycolysis may also result in increased oxidative stress, as heightened ROS levels can impair various glycolytic enzymes, consequently lowering glycolysis rates. The gene analysis of LDHA, PKM, ALDOA, and HK2, presented in FIG. 7C, aligns with these findings. These genes, crucial for regulating glycolytic function, are significantly downregulated, echoing the Seahorse experiment results. As mitochondria generate the majority of cellular ATP through oxidative phosphorylation, mitochondrial dysfunction can shift cellular metabolism from glycolysis to alternative energy-producing pathways. Further, Preeclampsia is linked to increased inflammation, which can impact cellular metabolism. Inflammatory cytokines can alter the expression and activity of essential glycolytic enzymes, potentially diminishing glycolytic activity. By examining the relationships between substrate stiffness, glycolysis, and ROS production, one can investigate how these factors influence cellular metabolism in the context of preeclampsia. Identifying the underlying mechanisms responsible for alterations in glycolytic activity and ROS levels may offer valuable insights for the development of targeted therapeutic interventions to manage preeclampsia.

Stiffer Substrates Elevate Oxidative Stress and Lower Glutathione in HTR8 Cells. Nrf2 Activation Impaired by Stiffness is Subsequently Improved by Sulforaphane Treatment

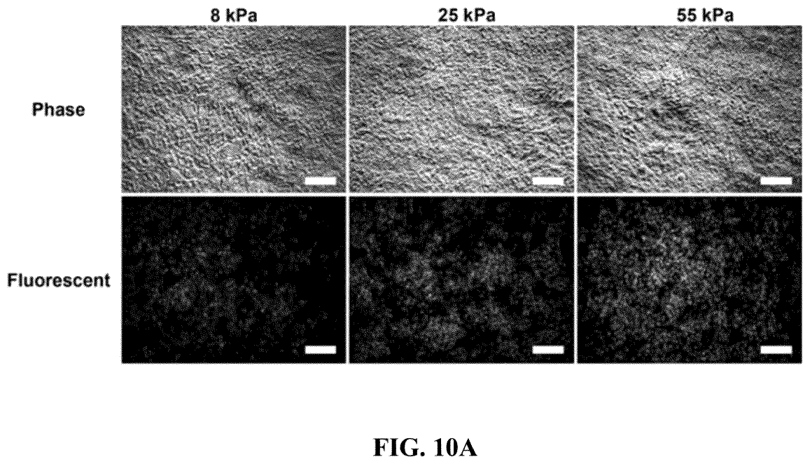

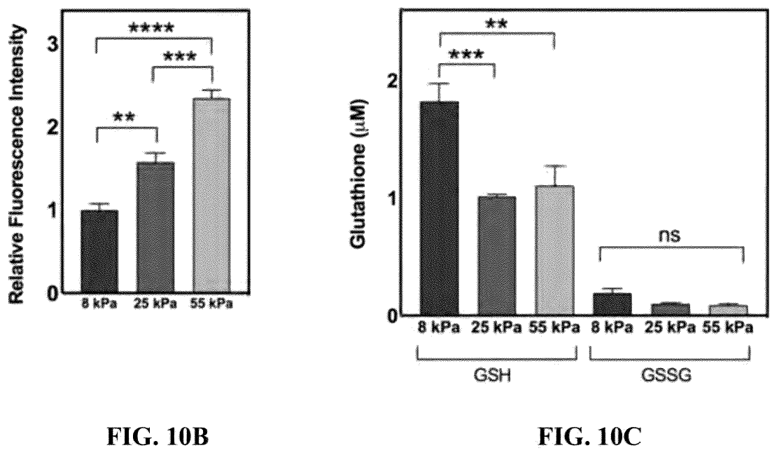

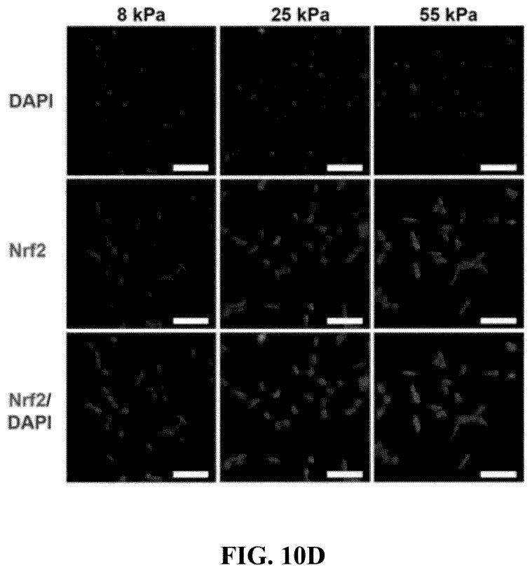

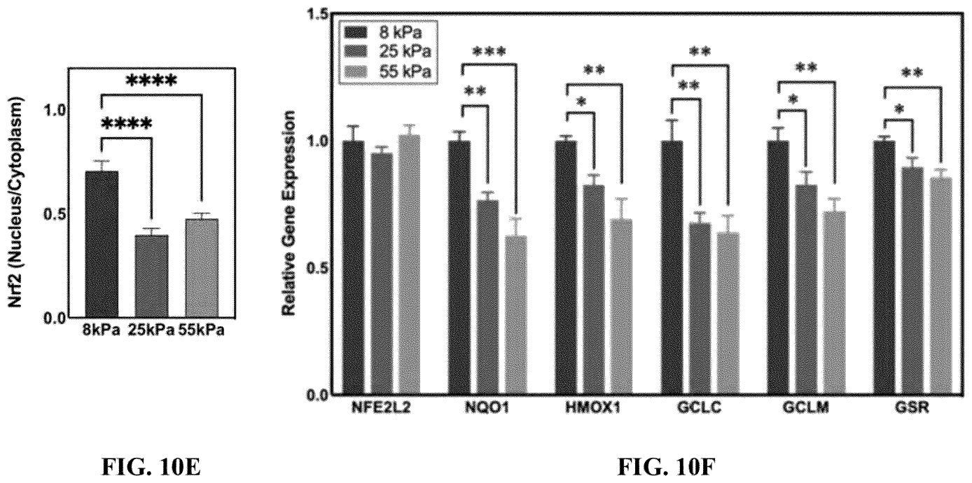

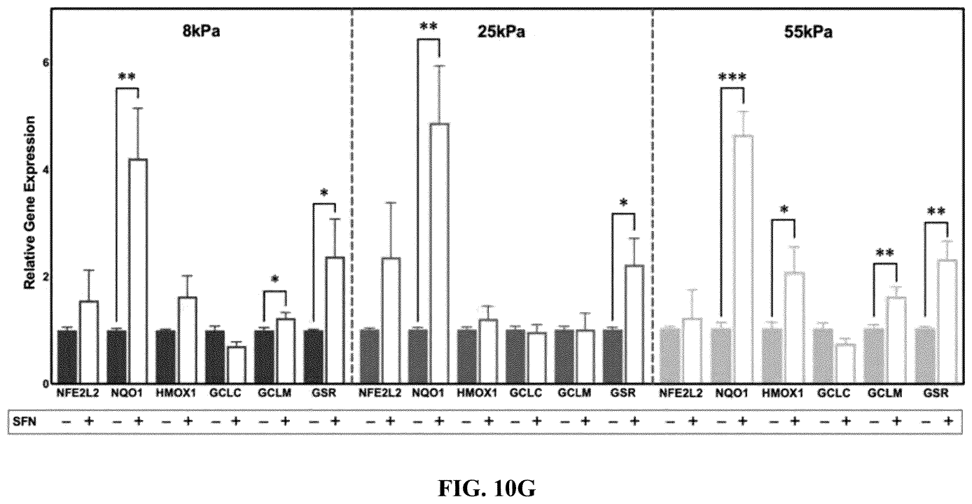

Preeclampsia is well-known for its association with oxidative stress, and data from both mitochondrial respiration (FIG. 9) and gene analysis (FIG. 7C) underscore the significant role of oxidative stress in preeclampsia and its relationship with substrate stiffness. To delve deeper into this association, ROS was analyzed using the H2DCFDA dye. Upon oxidation, the non-fluorescent H2DCFDA is converted into the highly green fluorescent 2′,7′-dichlorofluorescein. Cells were cultured on the BEASTS platform at three varying stiffness levels (8 kPa, 25 kPa, and 55 kPa) and treated them with the H2DCFDA dye after four days of culture (FIG. 10A). Subsequently, the cells were examined under a fluorescence microscope to evaluate ROS intensities, which revealed a higher fluorescent intensity as substrate stiffness increased. Using ImageJ for quantification, ROS intensity was confirmed to significantly escalate in parallel with substrate stiffness (FIG. 10B). This outcome aligns with preceding observations from the mitochondrial respiration analysis, reinforcing the connection between oxidative stress and substrate stiffness in the context of preeclampsia.

Levels of Glutathione were evaluated. Glutathione is a tripeptide antioxidant that plays an essential role in neutralizing the toxic effects of reactive oxygen species, including free radicals, lipid peroxides, peroxides, and heavy metals in eukaryotic cells. The reduced (GSH) and oxidized (GSSG) forms of glutathione cooperate with other redox-active compounds like NADPH to preserve and regulate cellular redox balance. The oxidized glutathione (GSSG) acts as a marker for cell health and oxidative stress. In this experiment, cells were cultured on BEASTS surfaces with varying stiffness levels for four days, followed by a GSH assay (FIG. 10C). There was a significant decrease in total reduced GSH levels at higher substrate stiffness, which is indicative of disease conditions. Interestingly, there were no substantial differences in GSSG levels among the three groups. This result could be explained by the elevated amounts of free radicals caused by increased oxidative stress at higher stiffness levels, as demonstrated in the preceding data. This increase in oxidative stress may have contributed to the observed reduction in glutathione levels.

Nrf2 activation was investigated, as it is a process closely linked to oxidative stress, which plays a crucial role in regulating the expression of antioxidant proteins. Under normal conditions, Nrf2 is bound to KEAP1; however, in the presence of oxidative stress, it dissociates from KEAP1 and translocates to the nucleus to initiate antioxidant responses. Studies suggest that nuclear Nrf2 concentration is increased by antioxidant exposure, but reduced by elevated oxidative stress. To explore Nrf2 levels, an immunostaining experiment was conducted using the BEASTS platform (FIG. 10D). Cells were cultured at stiffness levels of 8 kPa, 25 kPa, and 55 kPa for four days, after which they were stained with Nrf2 antibody (displayed in red) and DAPI for cell nuclei (shown in blue). Fluorescent images were captured using a confocal microscope to observe changes in Nrf2 concentration in the cytoplasm and nucleus, as well as its translocation from the cytoplasm to the nucleus, as substrate stiffness varied. Nuclear Nrf2 concentration was lower in higher stiffness conditions (25 and 55 kPa) compared to the 8 kPa healthy condition. Nrf2 translocation was quantified using ImageJ by evaluating the ratio of its fluorescence levels in the nucleus to those in the cytoplasm (FIG. 10E). A significant decrease in nuclear translocation was observed in cells cultured on stiffer substrates compared to those on the healthy 8 kPa substrate, consistent with the preceding findings that increased substrate stiffness leads to elevated oxidative stress in the BEASTS model. This observation was corroborated by the clinical data analysis (FIG. 7C) and a bar graph (FIG. 10F) that depicts changes in the expression of Nrf2-associated genes (GSR, GCLM, HMOX1, GCLC, NFE2L2, and NQO1) at different substrate stiffness levels, obtained from PCR experiments. The gene expression analysis demonstrated a significant decrease in the expression of these genes with increasing stiffness, supporting the finding of reduced Nrf2 nuclear translocation under these conditions.

Sulforaphane (SFN) is a naturally occurring compound present in cruciferous vegetables like broccoli, watercress, Brussels sprouts, cabbage, and cauliflower. Known for its anti-inflammatory and antioxidant properties, SFN has shown promise in mitigating the pathological mechanisms associated with cancer, lung injury, and preeclampsia. As a potent Nrf2 activator, SFN affects the expression of approximately 200 genes, including those involved in antioxidant and anti-inflammatory processes. To explore the impact of sulforaphane treatment on the expression levels of Nrf2-related genes, HTR8 cells were cultured on 8 kPa, 25 kPa, and 55 kPa BEASTS surfaces and the gene expression was analyzed by RT-PCR. There was an increase in the expression levels of these genes upon sulforaphane treatment, indicating enhanced Nrf2 activation. NQO1 and GSR were significantly upregulated across all three stiffness levels, while GCLM was markedly upregulated in 8 kPa and 55 kPa, and HMOX1 in 55 kPa. These genes have critical roles in the antioxidant response following Nrf2 activation: NQO1 and GSR function to reduce reactive oxygen species and detoxify harmful substances, HMOX1 produces antioxidant molecules and displays cytoprotective and anti-inflammatory effects, and GCLM is responsible for synthesizing glutathione, an essential cellular antioxidant. Sulforaphane can have therapeutic potential for reducing oxidative stress through the mediation of Nrf2 activation in the BEASTS platform. Notably, in cases of increased stiffness, such as 55 kPa, which simulates severe preeclampsia conditions, sulforaphane treatment can activate Nrf2, boost antioxidant responses, and potentially alleviate oxidative stress conditions influenced by substrate stiffness.

ROS intensities increased with substrate stiffness, while total reduced glutathione levels decreased at higher stiffness levels, indicating elevated oxidative stress. Nrf2 activation was found to be reduced under higher stiffness conditions, consistent with the observation of increased oxidative stress. Sulforaphane treatment showed potential in mitigating oxidative stress by upregulating the expression of Nrf2-related genes, suggesting a possible therapeutic approach for addressing preeclampsia-related oxidative stress. Overall, the data highlights the significant impact of substrate stiffness on oxidative stress in preeclampsia and underscores the potential of targeted therapeutic interventions to improve outcomes.

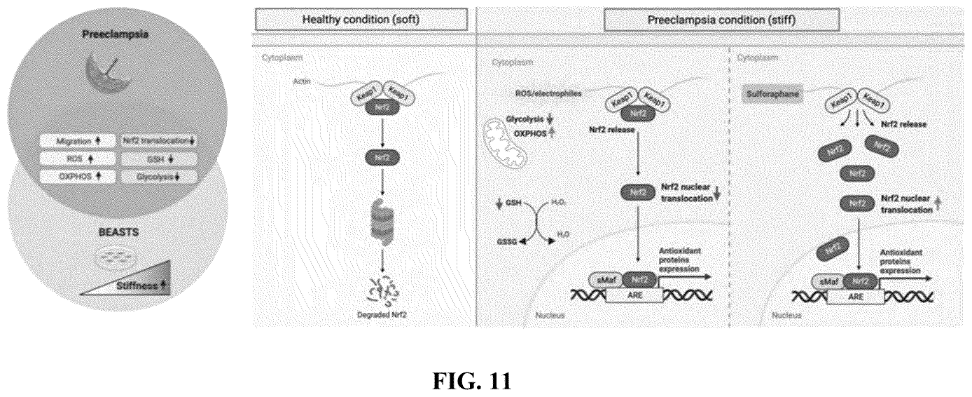

FIG. 11 is an illustration of the BEASTS model that effectively replicates cellular behavior and functions observed in preeclampsia using HTR8 cells. The model highlights increased oxidative stress levels and reduced glutathione concentrations on stiffer substrates that mimic PE conditions. The metabolic analysis uncovers a significant upregulation of mitochondrial respiration alongside a decrease in glycolysis function as substrate stiffness rises. Additionally, the translocation of Nrf2 is hindered under conditions of higher stiffness, which suggests a compromised antioxidant response. However, when treated with sulforaphane, Nrf2 activation is notably enhanced, as evidenced by the upregulation of relevant gene expression. This finding emphasizes the potential therapeutic benefits of sulforaphane in addressing PE-related cellular dysfunctions and improving antioxidant responses within the context of the BEASTS model. Consequently, this model proves to be an excellent tool for closely simulating PE conditions and observing stiffness-driven changes in cellular functions. It can potentially be utilized for further exploration of molecular mechanisms in trophoblasts related to PE and may serve as a foundation for identifying potential therapeutics for this condition.

### BEASTS Platform for Aging

The process of aging is accompanied by impaired tissue regeneration and repair mechanisms, resulting in tissue stiffening over time. These alterations in tissue stiffness are associated with an increase in stiff substrates, which in turn trigger inflammatory responses. In the central nervous system (CNS), microglia play a crucial role in regulating the primary immune response, and with aging, they become progressively activated and appear to undergo dystrophic changes. Data here demonstrated a remarkable accumulation of dysfunctional microglia in response to increased stiffness. These cells exhibit transcriptional signatures indicative of a disease-associated microglial phenotype, secrete higher levels of lactate dehydrogenase (LDH), accumulate lipid droplets, impair mitochondrial health, produce elevated levels of reactive oxygen species, and exhibit heightened inflammatory phenotypes in response to matrix stiffness. Stiffness may contribute to age-related neuroinflammation and an increase in disease-associated microglia. Pretreatment with n-acetyl-cysteine (NAC) mitigates stiffness-induced microglial proliferation and ROS production. Stiffness may contribute to age-related neuroinflammation and an increase in disease-associated microglia.

The brain is a unique organ in the mammalian body due to its softness, which is attributed to the extracellular matrix (ECM) that comprises mainly of glycosaminoglycans, proteoglycans, and chondroitin sulfate proteoglycans (CSPGs) instead of fibrous proteins. The ECM provides physical barriers, structural support, and regulation of various processes in the brain where neurons and glia reside. Cellular sensing and biochemical signaling of the ECM regulate physiological processes, and changes in the brain ECM occur during tissue repair, neurodegeneration, injury, and aging. A malfunction in the ECM's reparative process leads to pathological consequences, preventing normal brain function and resulting in detrimental outcomes found in fibrotic diseases.

Unlike other connective tissues, the brain's ECM is not abundant in collagen but is comprised of hydrated scaffolds such as glycosaminoglycans and glycoproteins. The brain's ECM regulates ion homeostasis, neuroprotection, and neuronal and glial function, which are gradually lost with age. Aging results in progressive tissue regeneration and repair defects, increasing oxidative stress in the brain and central nervous system (CNS), and affecting intracellular transport via regulation of the cytoskeleton. The cytoskeleton of neurons and glia connect to the ECM and transmit forces from the ECM to the cell's interior by mechanosensitive and Ca²⁺-permeable channels such as PIEZO1. Therefore, changes in composition, structure, and stiffness in the aged brain ECM alter cellular communication and may contribute to the progression of neurodegenerative diseases.

Microglia play a pivotal role in maintaining brain homeostasis and regulating immunological and nonimmunological functions. With age, microglia take on a primed phenotype with elevated expression of inflammatory markers and diminished expression of neuroprotective factors. Microglia appear to respond to variations in ECM stiffness as one key regulator of microglial phenotype and function. Brain ECM stiffness is a fundamental definition of fibrosis, which is frequently associated with aged tissue. How aged stiffness regulates phenotype and functional changes in microglia is poorly understood due to the challenge of controlling brain stiffness through animal models. Many in vitro models of aged brain stiffness have been met with limited success due to the nature of dependent non-biochemical factors introduced to stimulate immune cells to grow and divide. To address this challenge, a mechanically tunable protein-free scaffold, the BEASTS platform, was developed to recreate physiological and pathological stiffness without the need for growth factors to induce microglial cells to grow and divide.

Stiffness as one key regulator of microglial phenotype and function was evaluated using the BEASTS culture system. The effects of elevated stiffness on key microglial functions, such as proliferation, phenotype, inflammation, phagocytosis from lipid droplet accumulation, bioenergetics, and oxidative stress markers were examined using the HMC3 and BV2 cells as an alternative model for examining brain inflammation.

### Protein Expression in ≥40 Years and Older and <40 Years in Individuals Exhibits Varying Traits in the Prefrontal Cortex.

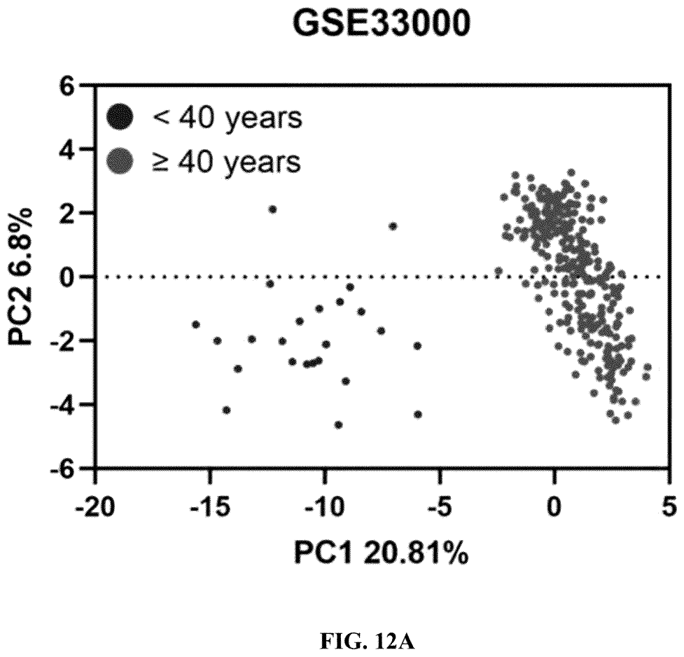

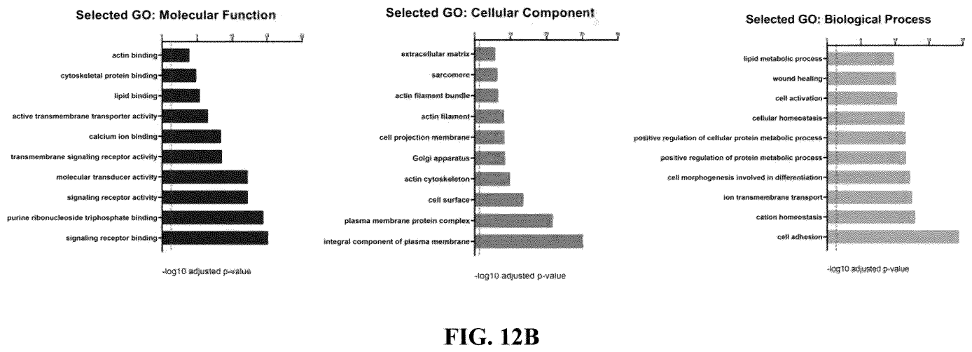

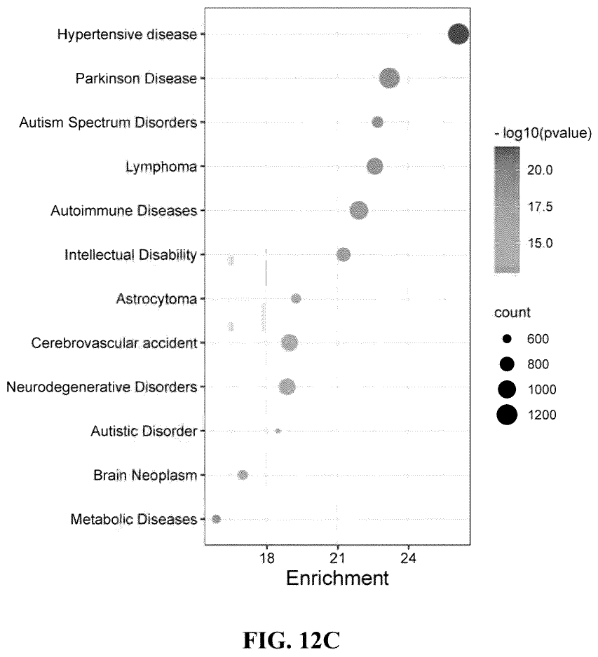

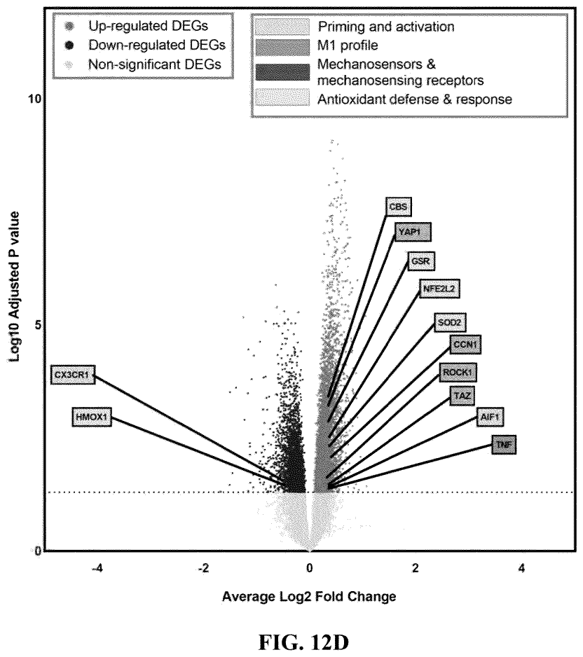

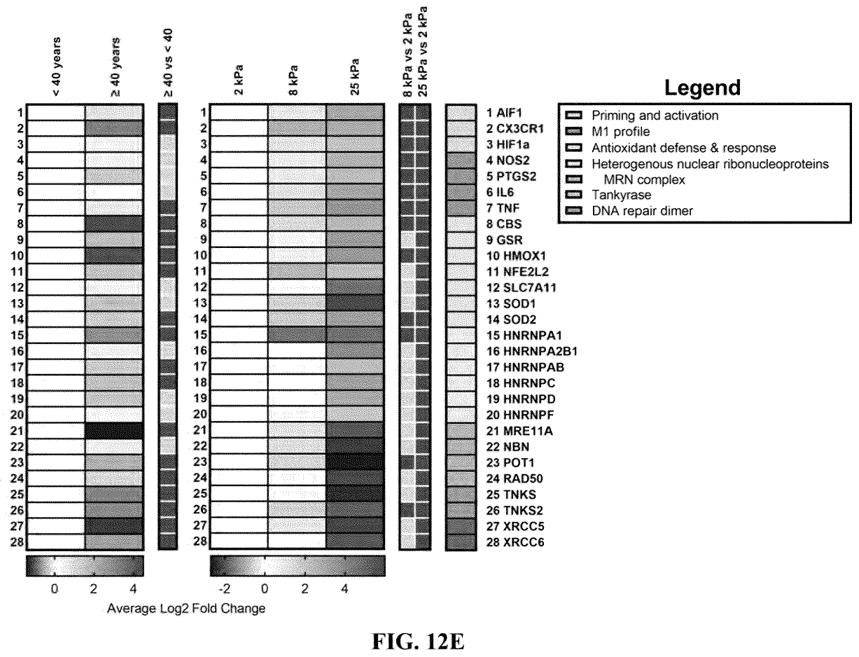

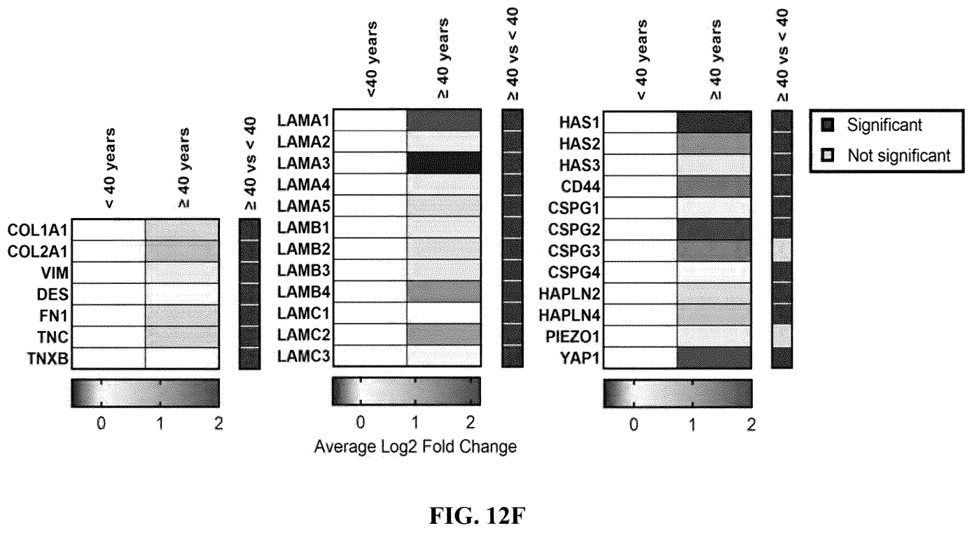

To gain insight into how the brain microenvironment might be impacted by aging, transcriptomic data collected across the prefrontal cortex from 624 individuals were analyzed. Whether aging might heighten the effect of the expression of extracellular matrix (ECM) components that may result in ECM protein deposition was investigated. Individuals were first classified into two groups: less than 40 (<40) and 40 years and older (≥40). Analyses were restricted to non-diseased young and aging tissues. Tissues from patients diagnosed with Alzheimer's Disease were excluded. Each tissue was matched with the reference tissue (see Methods) for each patient. The ≥40 and <40 samples had different populations from each other in Principle Component Analysis (FIG. 12A). These samples were further analyzed through gene ontology (GO) enrichment analyses. Relevant ECM processes in molecular function, cellular component, and biological processes were further analyzed (FIG. 12B). Next, a disease enrichment analysis was performed on ≥40 and <40 samples (FIG. 12C). Major pathways that were found to be involved are related to brain diseases with age abnormalities. The DEGs used in the GO and disease plot analyses are presented in a volcano plot in FIG. 12D. Genes were grouped as they are related to the priming and activation of microglia, MI macrophage profile, mechanosensory and mechanosensing receptors, antioxidant defense & response (ARE), and telomerase-associated genes (FIG. 12E). The ECM scaffolding proteins involved in the remodeling and resolution phases were further analyzed (FIG. 12F). These transcripts have been organized into three categories: fibrous proteins (FIG. 12F, left panel), laminins (FIG. 12F, middle panel), and specific brain ECM scaffolding proteins (FIG. 12F, right panel). These results suggest that the brain ECM is dysregulated with age showing patterns of dystrophic genotypes, and a correlation with age. The connection between ECM and age is unknown.

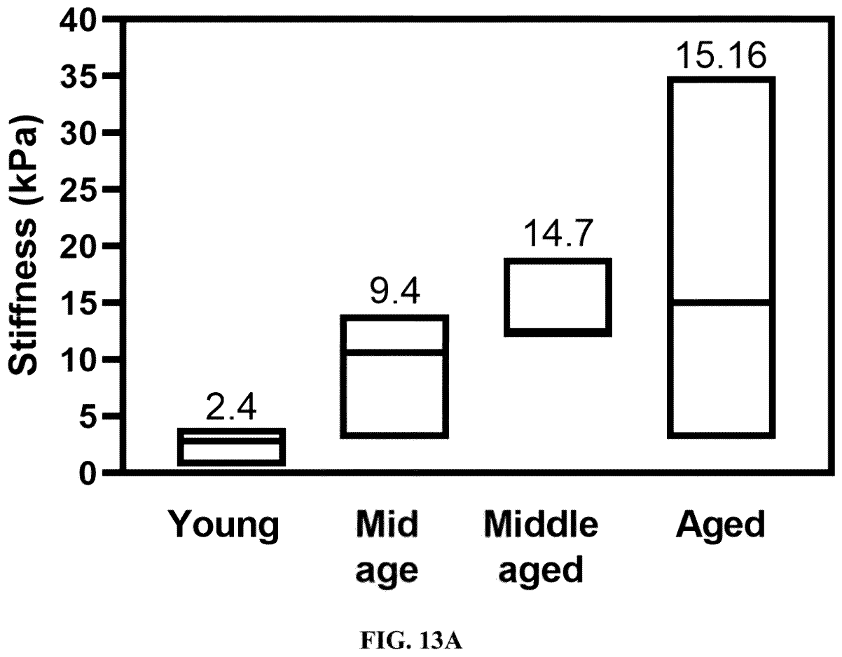

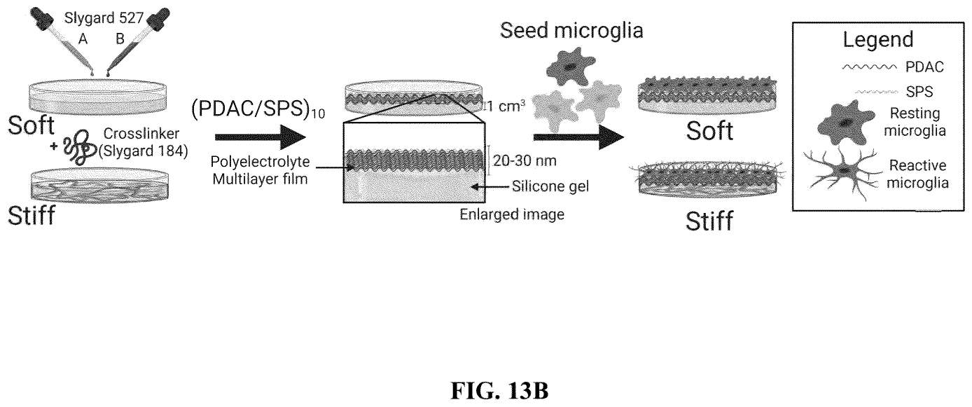

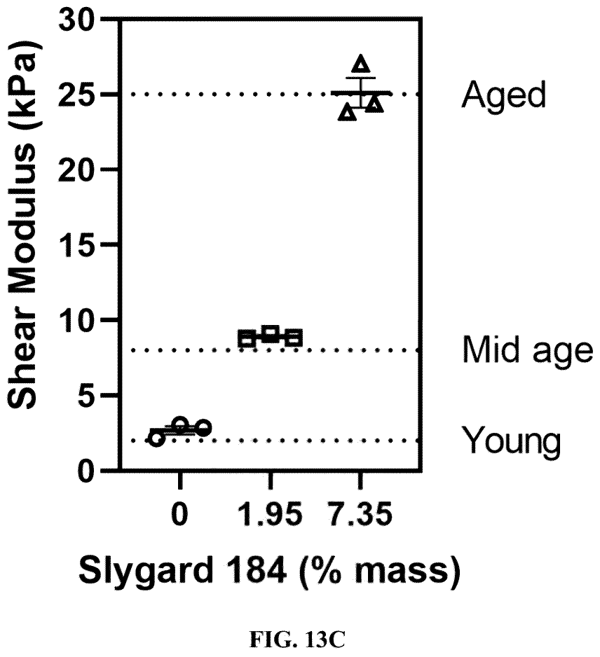

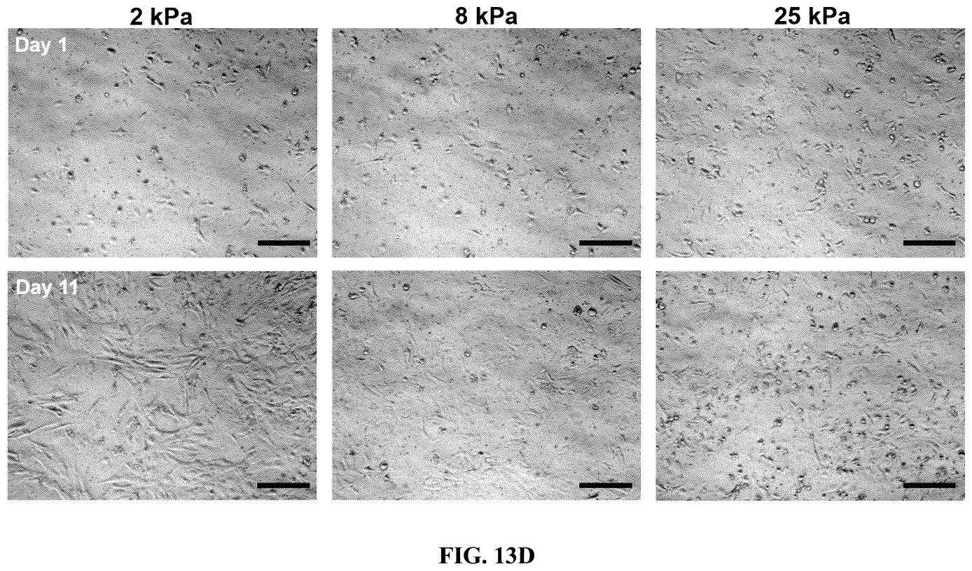

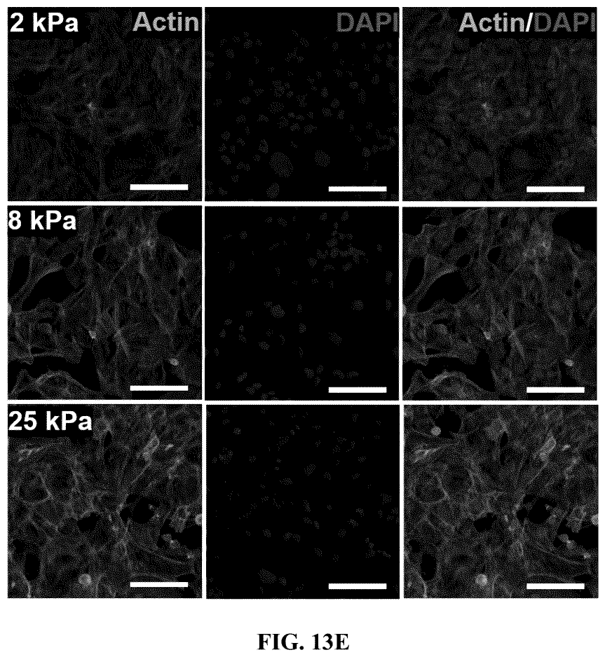

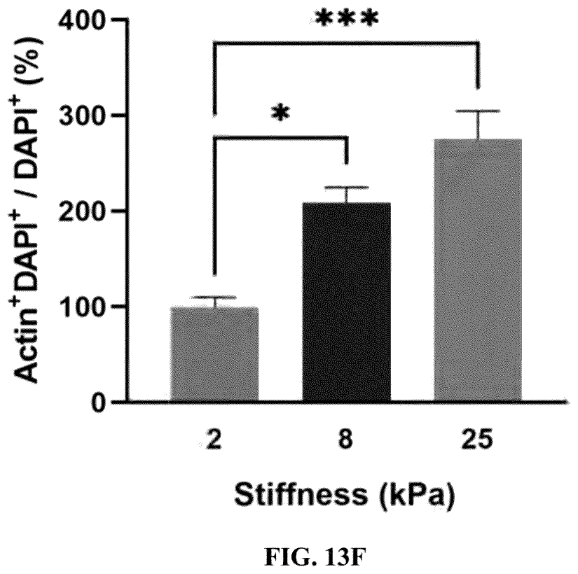

Recent evidence from in vivo imaging studies suggests brain tissue stiffness increases with age (literature values are presented in FIG. 13A. The effect of physical stress at the cellular level that could induce aging-like behavior was evaluated using the BEASTS platform. The BEASTS platform is based on PDMS substrate with Shear Modulus increasing with increased crosslinker to mimic aged brain tissue (FIG. 13B, Supplementary FIG. 2B). In combination with the polyelectrolyte multilayer film (PEM)-coating technology, a mechanically tunable substrate was engineered that allows cells to seed onto tissue mimics of both physiologic and pathologic stiffnesses (FIG. 13C). Three different platforms are created to model distinct stages of aging development based on prior ex vivo clinical specimens: healthy endothelial tissue (2 kPa; middle of 0.5-4 kPa, differences depending on tissue type), 8 kPa (mid-age), and 25 kPa (aged). Microglia were used to test the impact of varying stiffness mimicking physiological (2 kPa) and pathological stiffness (8 and 25 kPa).

The general morphology and the adhesion of microglia in primary cultures on both soft and stiff surfaces were examined. No difference was found between cells on differing surfaces after seeding (Day 0). Changes in morphology were found after 11 days on soft and stiff tissue mimics (FIG. 13D, Supplementary FIG. 2C). The microglia were then stained with Actin and counterstained them with DAPI to investigate the role of stiffness in determining changes in the structure and function of the cytoskeleton in fixed microglial cells. Increased stiffness increases the number of actin fibers present in microglial cells (FIG. 13E). The actin and DAPI channels were quantified as shown in FIG. 13F, finding a significantly increased actin signal in 25 kPa. These data suggest stiffness is a possible cause of effects with aging.

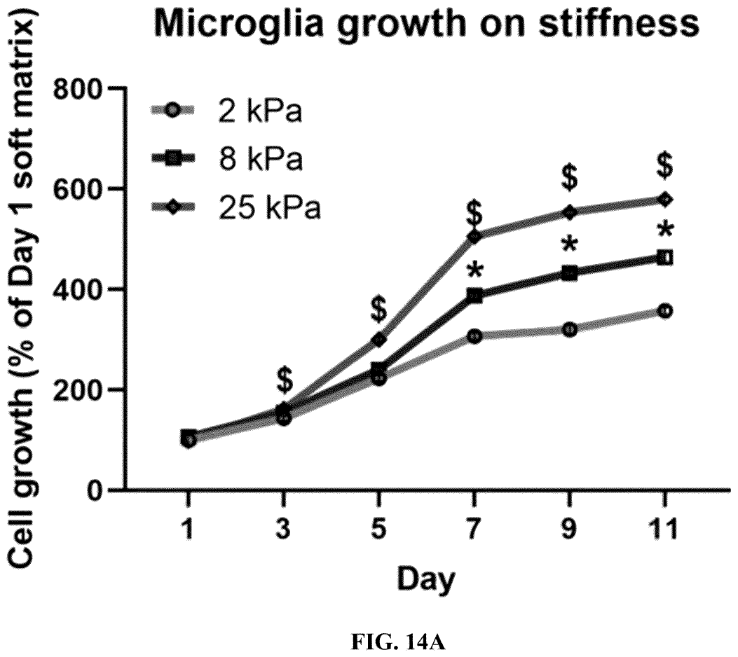

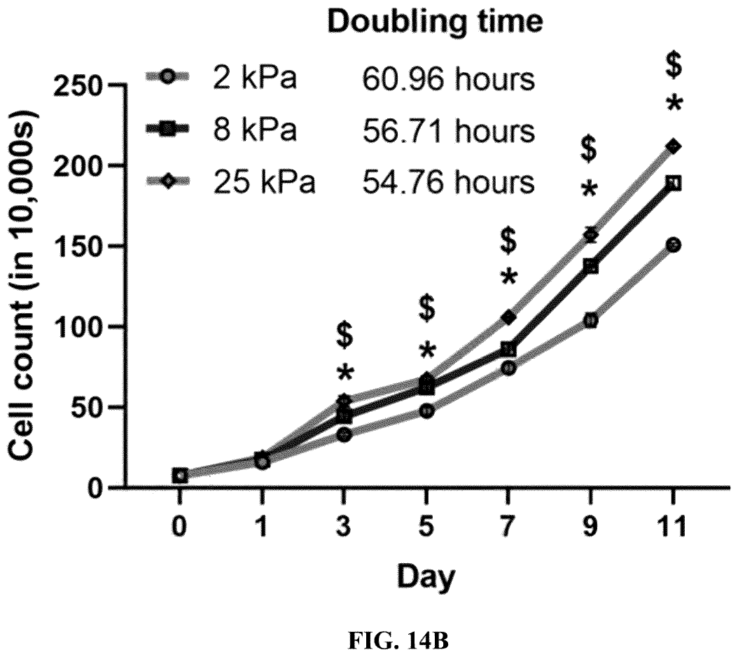

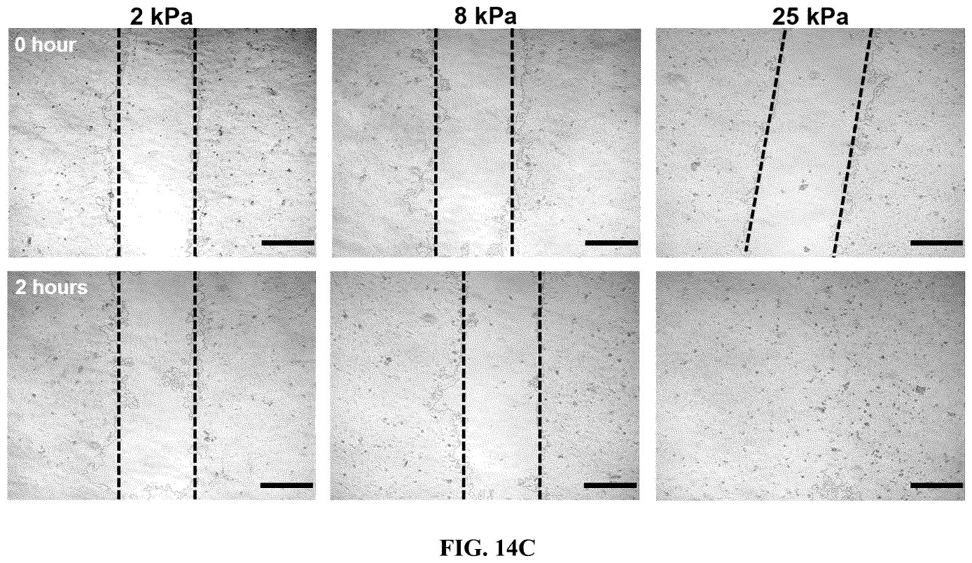

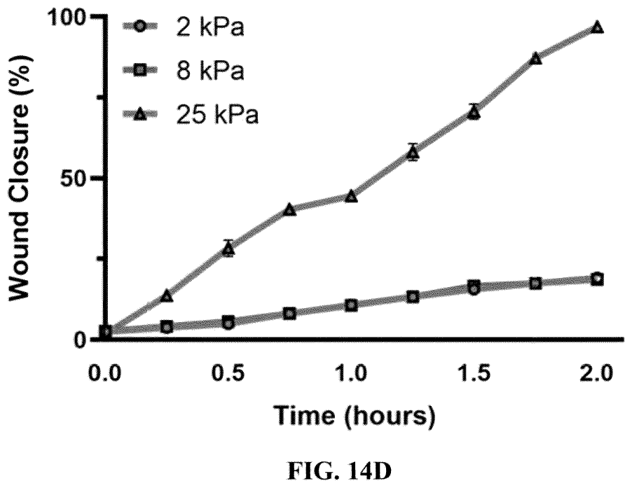

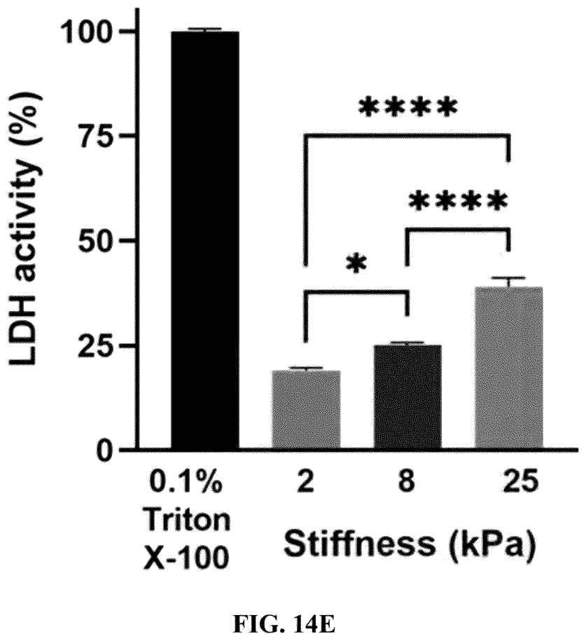

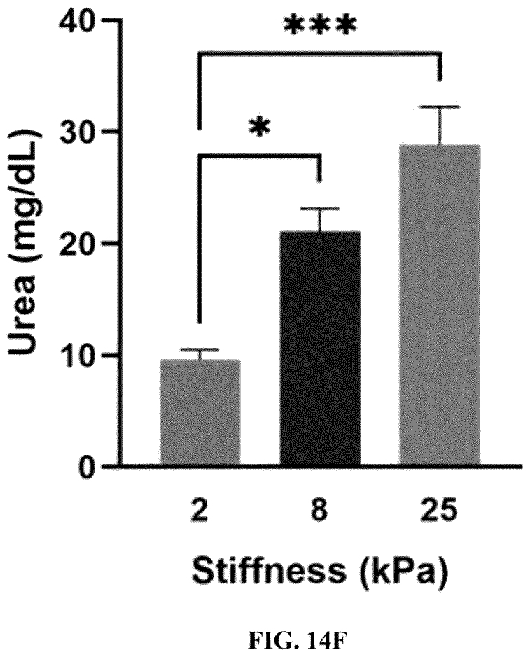

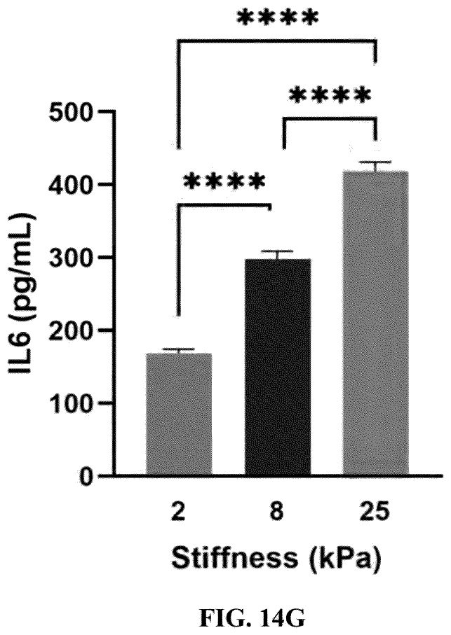

The hallmark traits of microgliosis and dystrophic genotypes were examined in microglia grown on soft and stiff tissue mimics. Proliferation and growth were assessed through MTT (FIG. 14A. Microglia have more growth on 25 kPa which was corroborated by the number of live microglia and doubling time as opposed to 2 kPa (FIG. 14B). At day 5 microglia began to grow significantly faster at >2 kPa and there was a reduction in doubling time with increasing stiffness (FIG. 14B). The morphology on stiffer substrates is akin to the microglial morphology observed from in vivo studies during microgliosis, with more surface area. Similar to the total cell count, microglia on day 5 and longer resulted in higher cell growth when grown on stiffer surfaces.

One of the early hallmarks of dystrophic microglia in aging is an increase in migratory properties in microglial cells. To measure the degree which microglial cells respond due to increased stiffness, a scratch assay was employed to assess microglial migratory properties on the BEASTS platform. The migration assay using a 200 μL pipette tip was chosen because of its reproducibility and the ease to induce a scratch without compromising the PDMS-coated surface. In a mere few hours, the microglial cells migrated to the artificially induced wound and repopulated the surface (FIG. 14C. The stiffer matrices significantly increased the migratory capacity of cells toward the scratch. The hardest matrix (25 kPa) closed the wound much faster than both soft matrices (2 and 8 kPa) (FIG. 14D). There was no difference in the morphologies between the soft and stiff microglial cells that repopulated the scratch area. Accelerated migration, increased proliferation, and enhanced priming and activation profile were observed in microglia (FIGS. 14A-14D). The release of pro-inflammatory mediators-specifically TNF, NO, and LDH51-from microglia were further assessed. Soft and stiff microglial cultures exhibit increased LDH release (FIG. 14E and urea (FIG. 14F) as well as IL-6 (FIG. 14G) and TNF. These studies corroborate the increased M1 inflammatory genotype (FIG. 12E) observed in aged individuals as well as microglia were replicated in the BEASTS platform. Stiffness plays a role in regulating the microglial inflammatory response.

### Stiffness Induces a Dysfunctional State and Lipid-Droplet Accumulating Microglia.

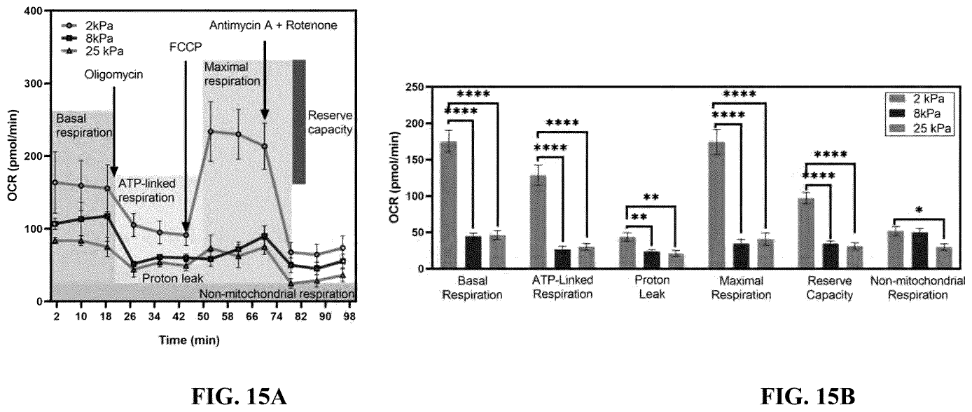

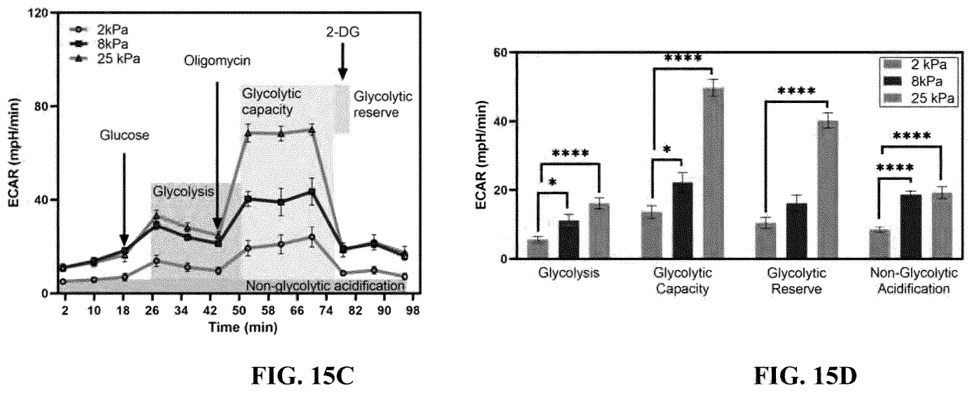

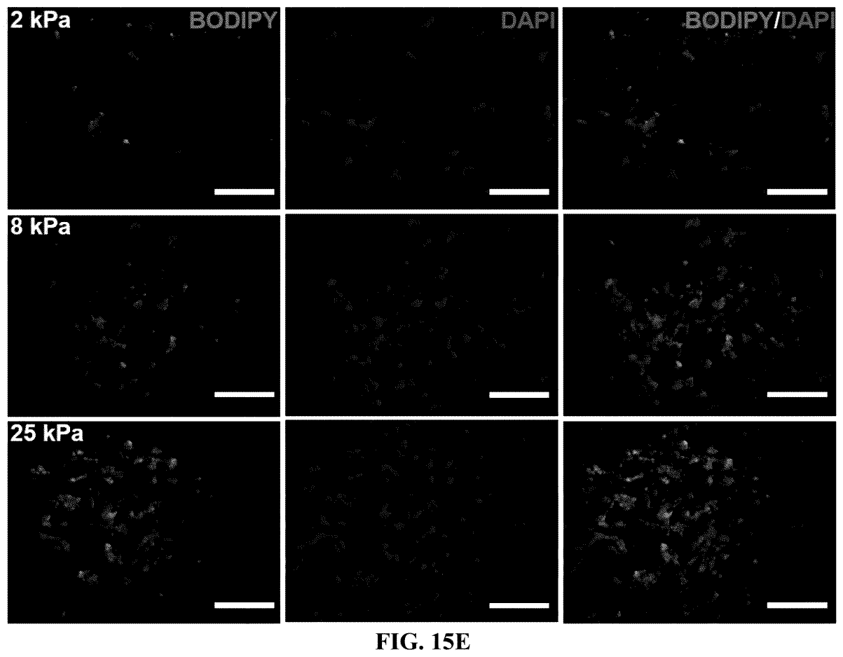

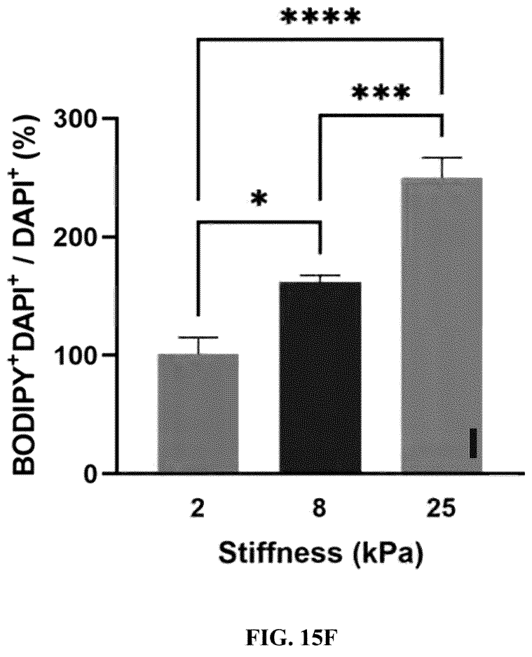

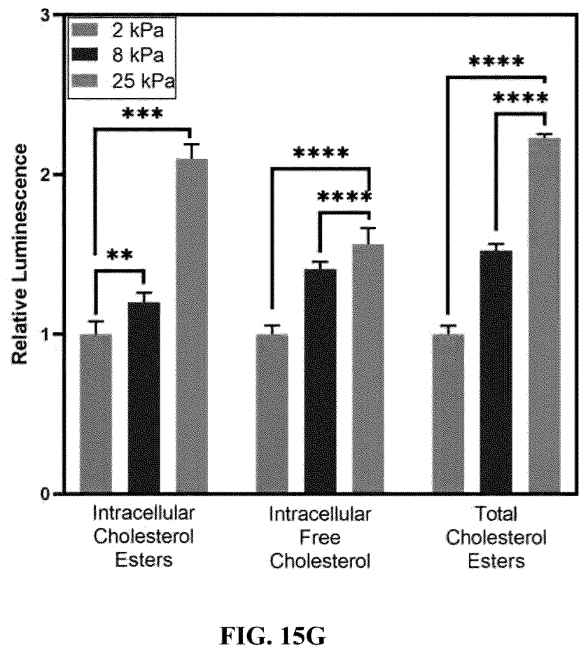

Under inflammatory activation (MI), microglia shift in energy metabolism from oxidative phosphorylation to glycolysis when presented with a pathological stimuli, a similar observation to the Warburg effect observed in cancer cells. To measure metabolic flux in real-time, the Seahorse XFe24 analyzers were utilized to measure the oxygen consumption rate (OCR) and extracellular acidification rate (ECAR) in microglia grown on soft and stiff tissue mimics. Microglia grown on 2 kPa utilize the electron transport chain (ETC), whereas microglia grown on 8 and 25 kPa have a lower overall OCR profile (FIG. 15A). The rates were quantified. Significant decreases were found in 8 and 25 kPa in basal respiration, ATP-linked respiration, proton leak, maximal respiration, reserve capacity, and non-mitochondrial respiration (FIG. 15B). In contrast, microglia grown on 2 kPa exhibited a lower ECAR profile than 8 or 25 kPa (FIG. 15C) and significant changes between microglial polarization. Strikingly, when the changes were quantified, an increase was found in the measurements of glycolysis, glycolytic capacity, glycolytic reserve, and an overall increase in non-glycolytic acidification with increasing stiffness (FIG. 15D). The changes in microglial metabolism drove the investigation into age-related metabolic defects observed in inflammatory microglia, with specific disease-related phenotypes.

One of the phenotypes found in dystrophic, aged microglia has aspects of disease-associated microglia (DAM) in chronic neuroinflammatory states with a build-up of fatty acids, cholesterol, and breakdown products. Specifically, cholesterol esters and fatty acids are significantly increased under microgliosis, with cholesterol esters-containing lipid droplets accumulating in late-onset Alzheimer's disease. To investigate the impact of stiffness on lipid metabolism, microglial cells were stained with 4,4-difluoro-4-bora-3a,4a-diaza-s-indacene (BODIPY), a dye that labels neutral lipids and detects lipid droplets. Interestingly, more amounts of lipid droplets were observed with increasing stiffness (FIG. 15E) similar to prior findings with aged microglia. The BODIPY signal was quantified. The stiff substrate mimics had significantly increased signal compared to 2 kPa (FIG. 15F). In an attempt to characterize the lipid droplets, the production of total cholesterol and cholesterol esters was quantified using a bioluminescent assay. An increase in intracellular cholesterol esters (FIG. 15G left), intracellular free cholesterol (FIG. 15G middle), and total cholesterol esters (FIG. 15G right) was detected, corroborating preceding finding of lipid droplet accumulation with stiffness (FIGS. 15E and 15F). Combined, these studies support prior findings of metabolic reprogramming in activated microglia and accumulation of lipid droplets in aged, dystrophic microglia with an inflammatory phenotype.

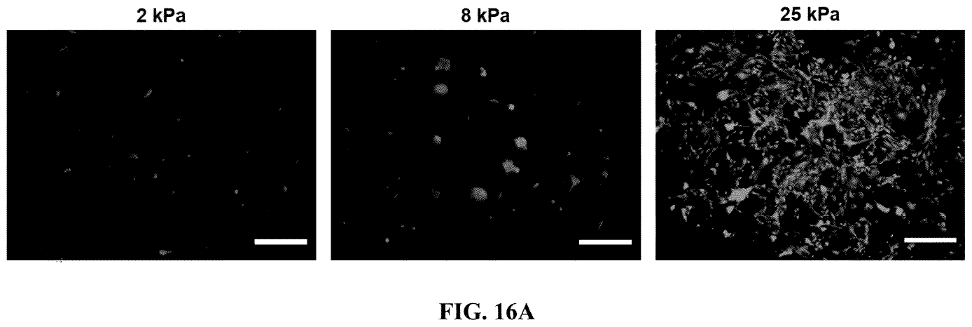

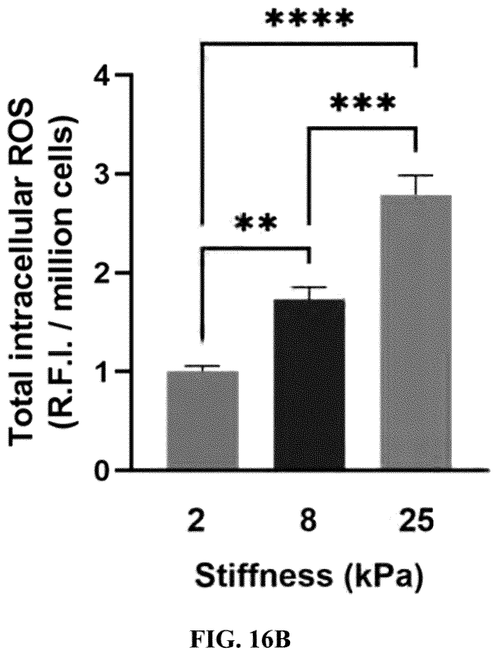

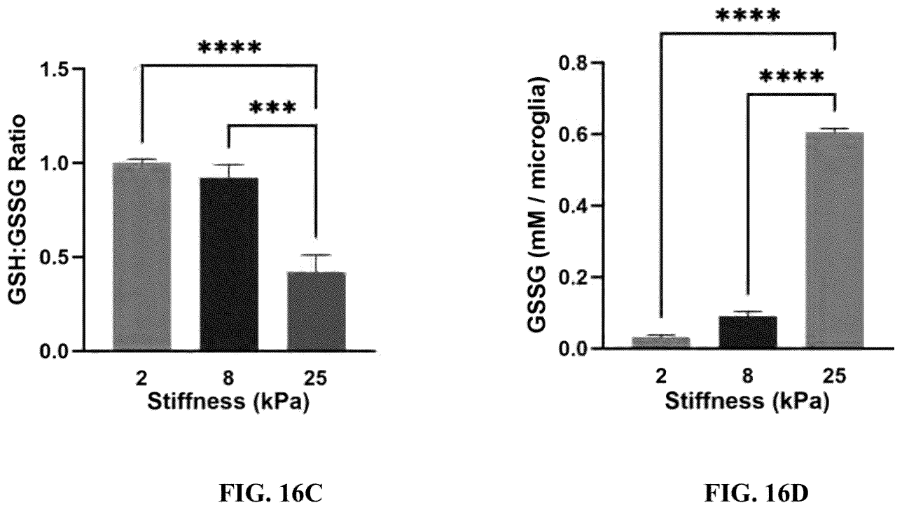

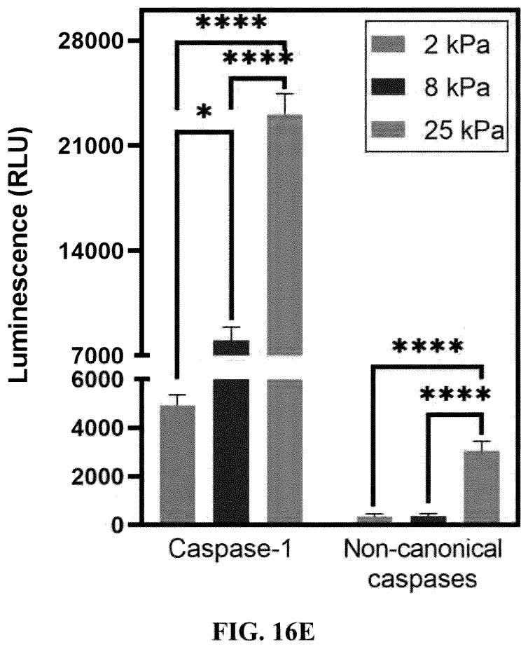

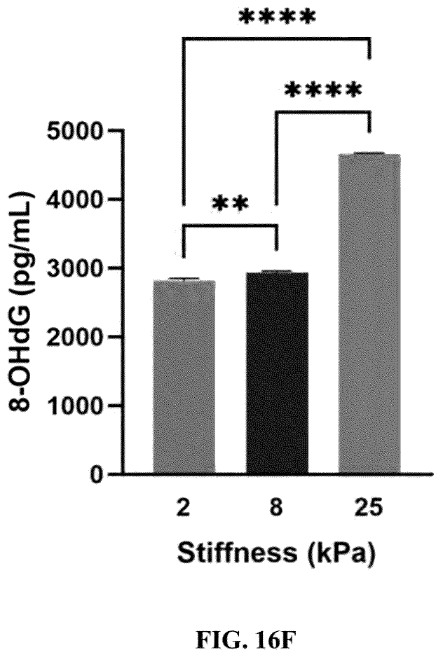

The effects of stiffness on microglial ROS production were examined to investigate the negative impact of increasing stiffness representing pathological tissue mimics. Total fluorescence using DCF-DA revealed that the levels of ROS gradually increased following treatment with 8 and 25 kPa stiffness (FIG. 16A). Stiffness alone significantly increased the production of ROS in primary microglia from 2 kPa and 25 kPa (p<0.001, multiple t-tests, corrected for multiple comparisons using the Bonferroni-Dunn method) (FIG. 16B). To investigate the impact of the increased ROS on the microglial redox state, the glutathione (GSH) pools that contribute by maintaining redox balance and protecting against oxidative stress were investigated. To determine the impact of stiffness-dependent activation of microglia, the redox status of the GSH:GSSG ratio was investigated. The GSH:GSSG ratio was significantly lowered in 25 kPa-grown microglia (FIG. 16C). This was corroborated by an increase in the total intracellular oxidized form of glutathione (GSSG) (FIG. 16D). The overexpression in ARE transcripts indicates oxidative stress in both aged brain tissue and microglia grown on the BEASTS platforms, as shown in FIG. 12E.

The increase in ROS and oxidative stress, along with the inflammatory phenotype, led to investigation of the NLRP3 inflammasome as this protein complex plays a critical role in the innate immune response. In microglia, oxidative stress can trigger the activation of the inflammasome, and the subsequent release of inflammatory cytokines. The caspase-1 activity was investigated as this member of the caspase family is known as the canonical caspase of the inflammatory response. Caspase-1 activity increases with stiffness (FIG. 16E). Additionally, non-canonical caspase activity was investigated by including a caspase-1 specific inhibitor, Ac-YVAD-CHO, to measure other cross-reacting caspases (5, inflammatory caspase; 3 and 6, apoptosis caspases). The activity of these caspase was greater in 25 kPa than in 2 or 8 kPa microglia. Thus, stiffness-driven inflammation is activated by caspase-1 in microglia, and the activity of non-canonical caspases, specifically apoptotic caspases 3 and 6, are activated on 25 kPa stiffness. As oxidative stress and inflammation are closely related processes, significant oxidative stress can cause DNA damage in inflammatory microglia. To measure DNA damage, a biomarker of oxidative stress leading toward DNA lesions, known as 8-hydroxy-2′deoxyguanosine (8-OHdG) was investigated. The 8-OHdG metabolite was present at higher concentrations on 25 kPa than 2 and 8 kPa (FIG. 16F), establishing a connection between the immune response and mechanically activated microglia. The stiffness-dependent activation of microglia induces more oxidative stress in microglial cells.

### N-Acetyl-l-Cysteine (NAC) Ameliorates Stiffness-Induced Dystrophic Microglial Phenotype.

Whether ROS is one of the drivers of the dystrophic phenotype observed in microglia grown on stiff substrates was further evaluated. NAC is a commercially available antioxidant that has been implemented in many clinical trials, including Parkinson's, Huntington, Alzheimer's, and amyotrophic lateral sclerosis diseases. The mechanism of action of NAC is by increasing sulfane sulfur species to stimulate mitochondrial bioenergetics, increase oxidant scavenging capacity, modulate protein function, and ultimately protect against irreversible oxidative damage.

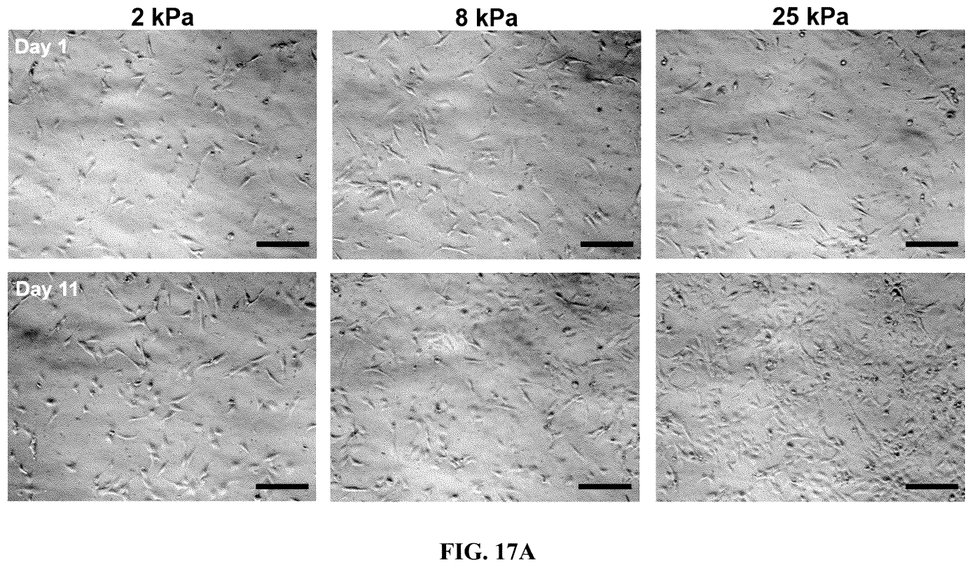

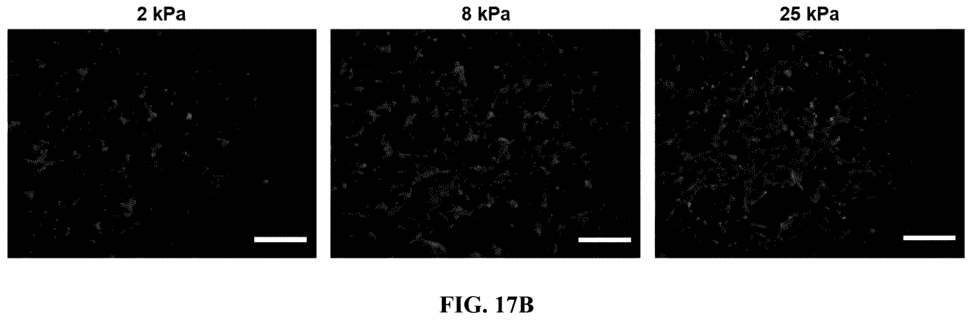

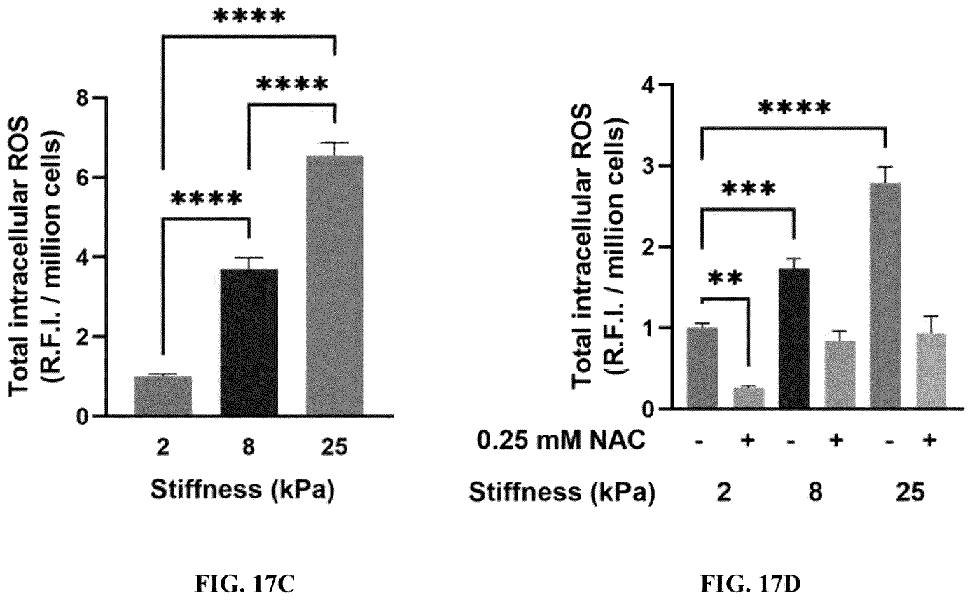

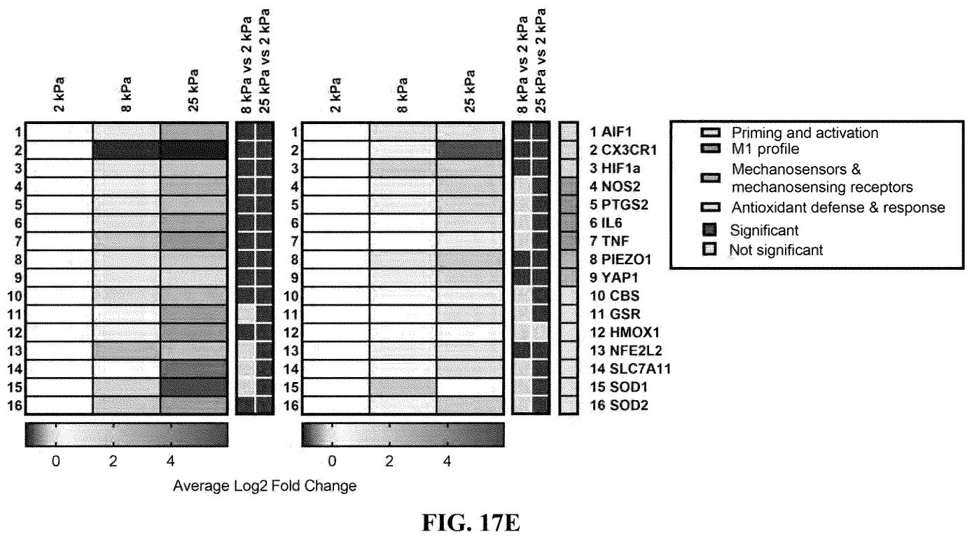

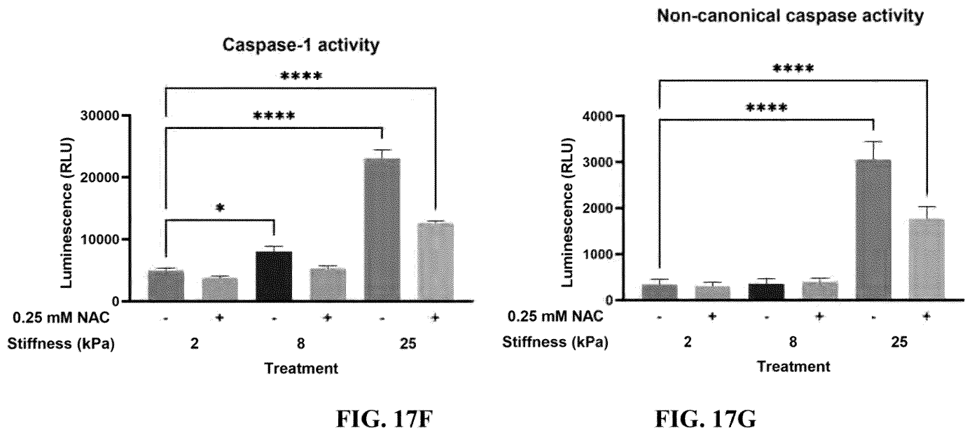

To investigate the protective effect of NAC on stiffness-activated microglia, microglial cells were pretreated with 0.25 and 2.5 mM NAC prior to seeding. After 11 days, the morphology was ramified across all treatments with 0.25 mM NAC (FIG. 17A). ROS production was then measured. There was an attenuation of ROS after 11 days, elevated levels with increased stiffness, and significantly reduced ROS production with NAC-pre-treated microglia (FIGS. 17B-17D). Next, the genotypes of untreated microglia were compared with NAC-treated microglia and an attenuation was found in each transcript. Interestingly, NAC-treated microglia still had a loss in the steady-state marker, CX3CR1, which decreased substantially on 25 kPa (FIG. 17E). Additionally the MI profile correspondingly attenuated and mechanosensors; however, the M2 marker PPARG increased. To investigate the influence of NAC on caspase-1 stiffness-driven inflammation, the activity of caspase-1 was measured. The activity was reduced in 2 and 8 kPa; however, 25 kPa had increased caspase-1 activity, corroborating the findings with the loss of the CX3CR1 transcript (FIGS. 17E and 17F). The activity of non-canonical caspases was also reduced in 2 and 8 kPa with NAC pre-treatment and elevated activity in 25 kPa which was attenuated with NAC (FIG. 17G). Together, these findings indicate that pre-treatment of NAC attenuates ROS-driven dysfunction in stiffness-activated microglia. Overall, these results demonstrate that stiffness activates microglia independent of ROS signaling and the redox state and drives an MI phenotype.

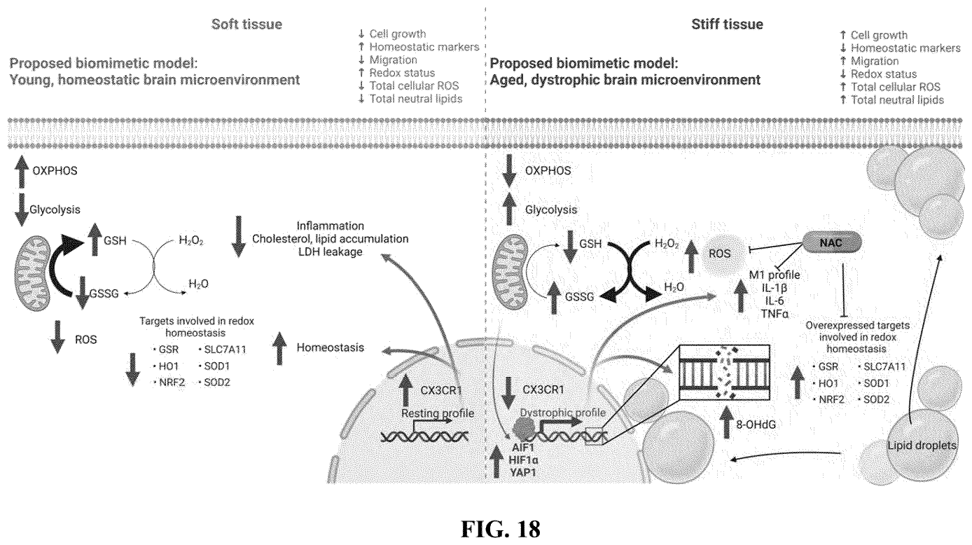

The BEASTS platform was robust to support the study of microglial states that mimic healthy and aging brain tissue. Data here shows that microglia interact with both tissue types with a unique transcriptional signature, severe functional defects, and a pro-inflammatory phenotype (FIG. 18). Although several in vitro models have been engineered to measure the impact of tissue mechanics on cellular behavior, these studies have rarely translated to native brain tissues. Therefore, an appropriate model of aged brain tissue is required to study and develop effective treatments for progressively aged tissue and related functional changes in microglia during aging. PDMS gels are the most commonly used polymer used in microfluidic or lab-on-a-chip applications. These gels can generate polymer substrates with high-spanning moduli but are not well suited for cell adhesion due to the hydrophobic nature of PDMS. However, cell-adhesive proteins such as fibronectin, laminin, and collagen have been used to encourage cell adhesion while also provoking immune responses, poor stability, and heightened cell responses not caused by mechanosensitive properties alone. Therefore, the BEASTS platform described herein presents a new appropriate aging tissue model showing stiffness-dependent responses from microglia on physiological and pathological stiffness.

Aging causes brain tissue repair and remodeling defects and is pathologically characterized by two causative factors, (1) namely the ecological niche to which cells adapt that causes acceleration and worsening of phenomena that characterize aging; and (2) specific interactions leading to an increase in inflammatory signaling and oxidative stress in the brain and CNS. An increased neuroinflammatory profile is primarily attributed to the resident immune cells, notably the microglia. Anatomical and functional decline progress in aged brains from microglia with dystrophic phenotypes. They are considered to be in a primed state and show an increased baseline production of inflammatory cytokines and become hyperactivated. Once hyperactivated, microglia show aberrant housekeeping properties such as lipid accumulation, migration, and ROS production. These changes are suggested to play a pivotal role in the clearance and disposition of debris in age-related neuroinflammation and functional impairments in the aged brain. These dystrophic phenotypes caused by the interaction of tissue mimics representing physiological and pathological stiffnesses suggest the microenvironment in aging may be one key factor leading to detrimental effects in the aging brain.

The exact triggers of underlying causes of increased stiffness of brain tissue and cross-linking of the extracellular matrix, the association between byproducts of metabolism linking ECM molecules, ROS, and inflammatory senescence, remain poorly understood. These dynamic tissue changes often form in response to inflammation, stress, and traumatic brain injury. Interestingly, stiffness provokes an inflammatory response in the microglia HMC3 cell line in vitro. Furthermore, pretreatment with NAC ameliorates the inflammatory phenotype and slows microglial growth. This is of particular interest because it has been shown that NAC can polarize microglia into the pro-healing or M2 phenotype. These findings are corroborated by the impact of PIEZO1, a mechanosensor, on the function of microglia and the resulting microglial activation. Thus, stiffness plays a key role in the activation and phenotype changes in microglial, and the BEASTS platform presents a model for age-related neuroinflammation. Besides neuroinflammation, metabolic changes toward stiffness have been reported to cause inflammatory responses in several immune cells. Interestingly, genes associated with the “priming and activation” pathway are significantly upregulated in microglia. For example, expression of AIF1, a protein that shortens the cell cycle, alters cyclin expression, and is involved in motility-associated rearrangement of the actin cytoskeleton, was significantly higher in microglia grown on stiffer matrices than in microglia grown on 2 kPa. Similarly, PIEZO1, a calcium channel that opens under membrane tension, was significantly overexpressed on stiffer matrices than 2 kPa. Moreover, these microglia showed a significantly lowered mitochondrial respiratory phosphorylation. These findings suggest that there is a switch in the bioenergetic profile due to stiffness.

Increased ROS generation is one key characteristic of aged microglia and is observed in prior studies. Reports of whether ROS is a cause or consequence of accelerated aging are contradictory. Interestingly, the in vitro results here demonstrate that pretreatment of NAC ameliorates stiffness-induced ROS in HMC3 cells, which supports the idea that aging tissue mimics have a causal role in the stiffness-induced generation of ROS. It is also possible that elevated ROS initially triggers the induction of microglial activation, and subsequently, stiffness induces ROS formation and exacerbates intracellular ROS load.

NAC pretreatment showed a transition from amoeboid to ramified morphology in 0.25 mM and an almost complete transition to ramified after 11 days on stiffness. This finding is in line with a previous report which observed that NAC pretreatment inhibits TAZ (transcriptional co-activator with PDZ-binding motif)S-glutathionylation and another showing NAC abrogates YAP1 (yes-associated protein), both important transcriptional co-activators downstream of the hippo pathway. As a mechanosensor, YAP has expression independent of the canonical Hippo pathway in the presence of increased ROS, promoting inflammation in macrophages grown on stiff hydrogels and a reduced YAP expression on soft hydrogels. NAC functions as an antioxidant are known by triggering intracellular H2S and sulfane sulfur production. The exact mechanism of how NAC might interfere with stiffness remains to be shown. In this context, a comparative study on aging macrophages treated with similar concentrations of NAC shows similar improvements in ROS production in both in vivo and in vitro treatment with NAC. Notably, the qRT-PCR analysis revealed that mechano-inflammatory signals PTGS2 and YAP1 expression values decrease with NAC pretreatment, in which PTGS2 is known to directly regulate YAP1 expression.

Recently, it has been shown that several subsets of microglia are present in both healthy and aged brains. The aging transcriptional signature overlaps with a regulated reciprocal direction with increasing stiffness. Furthermore, SOD1, SOD2, and other key antioxidant genes that are identified as neurodegenerative disease-associated markers have been found to be upregulated with stiffness and present in dystrophic microglia. Likewise, CX3CR1 was downregulated, a characteristic that supports stage I of disease-associated microglia. Stiffness also perpetuates dysfunctional phenotypes, while NAC corrects for imparities associated with stiffness. Therefore, the BEASTS platform presents a robust model that mimics both physiological and pathological brain tissue and is suitable for preclinical treatment studies in aging research.

### Materials and Methods

Cells: PHHs from commercial sources that are pooled from different donors are used. The use of commercial PHHs has two advantages: a) robust quality control and large-scale banking (via cryopreservation) from commercial sources mitigates the functional variability in PHH lots; and b) it enables robust dissemination of the protocols and platforms to other labs in the future. All the preliminary data were generated from PHHs from at least three different pooled batches (FIGS. 3A-3C). The cells will be cultured in serum-free media to ensure that serum does not impact the metabolic function of PHHs. The human cell line-LX2 is used for HSC experiments.

PHH culture experiments: PHHs are cultured on the BEASTS platform, which possesses the ability to sustain a long-term expression of the ethanol-metabolizing enzymes CYP2E1 and ADH for up to 10 days (FIGS. 3A-3C). Specifically, five different stiffnesses are used to model distinct stages of fibrosis based on Fibroscan measurements of CLD patients: healthy (2 kPa-soft), fatty liver (8 kPa), steatohepatitis (15 kPa), fibrosis (25 kPa), and cirrhosis (55 kPa-stiff). Cells on a substrate with 2 kPa (healthy) stiffness are used as controls.

Functional validation: The PHHs cultured on the BEASTS platform are validated for function through biomarker analyses. Media samples from the cultures are collected every day for 30 days. Secreted albumin, ceruloplasmin, alpha-1 anti-trypsin (A1AT), and transferrin in the supernatant are measured by ELISA. These concentrations are used to calculate the secretion rates (ng/h) of the four biomarkers. mRNA levels are examined for hepatocyte-specific markers (ALB, CY3A4, GSTA1, and SLCO1B1). In addition, the PHHs are subjected to staining procedures every week for live/dead staining and immunostaining against liver biomarkers including Mac1, E-cadherin, ZO-1, cytokeratin 18, albumin, and CYP450.

Ethanol metabolism: At the end of each incubation period, the culture media are collected for measurement of ethanol metabolism, i.e., residual ethanol and metabolically derived Ach is measured by gas chromatography. Acetate is measured using a commercial kit. Cells are scraped into sterile PBS to obtain cell pellets for RNA extraction and for measurement of protein expression by WB analysis. The contents (by WB) and catalytic activities of the ethanol-metabolizing enzymes ADH, CYP2E1, and ALDH2 are measured.

Hepatocyte function and injury: Albumin is a measure of hepatocyte function and alanine aminotransferase (ALT) and lactate dehydrogenase (LDH) are a measure of hepatocyte injury (necrosis). To determine if ethanol potentiates hepatocytes to contribute to liver inflammation akin to the clinical data, inflammatory mediators are measured, including the proinflammatory markers TNF-α and IL-6, the anti-inflammatory marker IL-10, and the chemotactic protein MCP-1, by WB and RT-PCR. ALD also results in increased triglyceride (TG) accumulation in hepatocytes. TG accumulation is measured in lysed hepatocytes cultured under different stiffnesses using an enzymatic assay kit from Stanbio (San Diego, CA). These values are normalized to total protein in the extract measured with the bicinchoninic acid (BCA) method. Apoptosis is measured in terms of caspase-3 activity and intracellular ATP levels. Functional genes are measured, including phase I/II enzymes and influx and efflux transporters. The enzyme activity of selected cytochrome P450 family enzymes (CYP1A1/2, CYPB1/2, CYP3A4, and CYP2D6), nicotinamide N-methyltransferase (NNMT), NTCP (a sodium-dependent bile acid transporter), bile salt export pump (BSEP), and CK18 are also determined. Hepatocytes express asialoglycoprotein receptor (ASGPR), which specifically mediates the endocytosis of non-sialylated glycol conjugates, such as alpha-acid glycoprotein (ASOR) and fibronectin (adhesive protein). ASGPR expression has been clinically correlated to hepatic function in patients with liver diseases, and the binding of anti-ASPGR antibodies was found to be practically absent in ALD liver tissues. Reduced ASGPR expression has been associated with impaired clearance of exogenous desialylated glycoproteins in ALD rodent models. PHHs are incubated in the presence or absence of I-ASOR with and without cold competitor to evaluate receptor expression and endocytosis. If required, an optimal synthetic plating surface with the addition of secreted growth factors such as HGF is used to prolong the ASGPR expression and endocytic activity of the hepatocytes.

Statistical Analysis: The data were presented as the mean±standard error of the mean (SEM) for each group's replicates. To determine the differences between the experimental groups, a one-way analysis of variance (ANOVA) was conducted, followed by Turkey's multiple comparison test using GraphPad Prism software. A p-value of less than 0.05 was considered significant for all statistical analyses. GraphPad Prism or STAT VIEW-J 5.0 is used.

RNA Seq Analysis for DEG Identification and Enrichment: The RNA sequencing data was obtained from UNMC (Dr. Berry's lab) using tissue samples collected from both healthy and preeclamptic women, with four samples in each group. To identify differentially expressed genes (DEGs), GraphPad Prism was employed and t-tests were conducted to determine genes that exhibited statistically significant alterations. The threshold for identifying DEGs was set at a P-value<0.05.Principal Component Analysis (PCA) was carried out using GraphPad Prism, which demonstrated distinct clustering of healthy and preeclamptic samples based on their gene expression profiles. In order to gain a better understanding of the functional implications of these

DEGs, the ToppGene tool for Gene Ontology (GO) term and pathway enrichment analyses was utilized. This comprehensive analysis provided insights into the biological processes and pathways that are significantly impacted in the context of preeclampsia.

Culture of HTR8/SVneo cells: HTR8 cells were cultured in RPMI-1640 Medium (Fisher Scientific), supplemented with 5% fetal bovine serum and 1% antibiotic-antimycotic solution (100×) (Gibco) and were incubated at 37° C. and 5% CO2. The cells were initially seeded at 25% confluency, and the culture medium was replaced every two days to maintain optimal growth conditions. When the cells reached approximately 90% confluence, they were subcultured and passaged by dissociation using a 0.25% Trypsin-EDTA solution. The cells were then split in a 1:3 ratio to ensure appropriate growth density and ample space for continued proliferation.

Fabrication and Characterization of certain BEASTS platforms: Sylgard 527 and Sylgard 184 (Fisher Scientific, USA) were combined in various weight ratios to produce PDMS substrates with desired stiffness for culturing HTR8 cells. Initially, Sylgard 527 was prepared by mixing equal weights of part A and part B to facilitate cross-linking and ensure homogeneity. Similarly, Sylgard 184 was prepared by mixing the elastomer and the crosslinking agent in a 10:1 ratio. The two Sylgard precursors were then combined in weight ratios of 3:97, 9:91, and 15:85 (Sylgard 184: Sylgard 527) and poured into 12-well tissue culture plates. These plates were cured overnight at 65° C. in an oven. Subsequently, the cured gels underwent plasma treatment for 8 minutes, during which negatively charged oxygen ions were deposited on their surface. The gels were then coated with 10 bilayers of positively charged PDAC polymer and negatively charged SPS polymer. Each layer was incubated with the gels for 20 minutes, followed by a deionized water wash. The plates were UV sterilized overnight before cell seeding. The elastic modulus (stiffness) of the PDMS substrates was measured using a TMS-Pro texture analyzer from Food Technology Corporation (Sterling, VA, USA). The analyzer compressed each sample by 0.2 mm and collected the applied force and displacement data to calculate the elastic modulus. The linear region of the stress-strain curve relationship was used for the calculations. To ensure accuracy, three independent samples were tested for each substrate type, and the results were averaged.

Gene Expression analysis of the HTR8 cells: Cells were cultured on BEASTS substrates of different stiffness levels (8 kPa, 25 kPa, and 55 kPa) for four days. After the culture, the total RNA was isolated from the HTR8 cells using Trizol following the manufacturer's instructions. RNA was extracted from the aqueous phase of the cell lysates by adding chloroform, followed by

RNA precipitation using isopropyl alcohol and rinsed with 75% ethanol. The RNA pellet was quantified and quality-checked using a spectrophotometer and then reverse-transcribed using the iScript™ cDNA synthesis kit. Subsequently, the relative expression levels of the target genes were analyzed using quantitative real-time PCR with SYBR Green Master Mix, and GAPDH was used as the housekeeping gene. The AACT method was employed for data analysis.

Western Blot: Cells were cultured on BEASTS surfaces with varying stiffness levels (8 kPa. 25 kPa, and 55 kPa) for four days. Following this, whole cell lysates were collected using RIPA buffer (PBS, pH 7.4, 1% CA 630 IGEPAL, 0.5% deoxycholate, 0.1% sodium dodecyl sulfate, protease inhibitor cocktail, and phenylmethylsulfonyl fluoride) [Sigma Aldrich]. To quantify the total protein concentration, the BCA protein quantification kit from Abcam (Cambridge, MA, USA) was utilized. The samples were then run on a 4-20% Mini-PROTEAN® TGX™ Precast Protein Gels to separate the proteins. After transferring the separated proteins onto a membrane, it was probed for the presence of GPR18 (Thermo Fisher). Protein bands were quantified using ImageJ software, and the obtained values were normalized with respect to the total protein concentration in the 8 kPa sample. This analysis provided insight into the expression of GPR18 in cells cultured on BEASTS surfaces with different stiffness levels.

Actin staining, cell size, and circularity analysis: Cells were cultured on BEASTS surfaces of different stiffness levels (8 kPa, 25 kPa, and 55 kPa) for four days, fixed using 4% paraformaldehyde in PBS at room temperature for 30 minutes, and permeabilized with 0.2% Triton X-100 for 15 minutes at room temperature. Actin 488 Ready Probes (Life Technology) were used to label the cells following the manufacturer's instructions and incubated for 20 minutes at room temperature. DAPI was used to stain the cell nuclei by incubating the samples in a 1 mg/ml solution for 10 minutes at room temperature. Confocal microscopy was used to acquire fluorescent images with appropriate FITC and DAPI filters. Using ImageJ software, cell area, and circularity were quantified from the fluorescent images, where 15 random cells were selected per image and their area and circularity were analyzed. The cell area was normalized to the average cell size on the 8 kPa surface, and circularity was represented as a unitless value ranging from 0 to 1, with 1 representing a perfect circle.

Proliferation assay: HTR8 cells were seeded in 12-well plates containing BEASTS gels with varying stiffness levels (8 kPa, 25 kPa, and 55 kPa) at 25% confluence. After every 24-hour time period, the cells were detached using 0.25% trypsin and counted using a hemocytometer. This process was repeated for four consecutive days until the cells reached confluence in the cell culture wells. Following this, the doubling time for each stiffness condition was determined to assess the growth dynamics of the HTR8 cells in response to the different substrate stiffnesses.

Cell migration assay: HTR8 cells were seeded in 12-well plates containing BEASTS gels with varying stiffness levels (8 kPa, 25 kPa, and 55 kPa) at a density of 0.5×10⁶ cells/well in 1.0 mL of complete medium. When the cells reached 95% confluence, a sterile pipette tip was used to gently create a scratch across the center of the well, simulating a wound in the cell monolayer. The scratched cells were then incubated at 37° C. and 5% CO2 until the wound closed in one of the experimental groups. Phase-contrast images were captured at different time points, specifically at 24 hours and 36 hours, to monitor the wound closure process. The cell migration rate was determined by calculating the area covered by the cells over time using ImageJ software to analyze the images. Multiple independent experiments were conducted, with approximately 6 to 10 replicates for each stiffness level.

Mito stress test assay: Cells were cultured on BEASTS surfaces with varying stiffness levels (8 kPa, 25 kPa, and 55 kPa) for four days and then transferred to XFe24 culture plates. After a 24-hour incubation period, the growth medium from each well was removed, leaving 50 mL of media. Cells were washed twice with 1,000 mL of pre-warmed assay medium (XF base medium supplemented with 25 mM glucose, 2 mM glutamine, and 1 mM sodium pyruvate; pH 7.4), and 1,000 mL was removed as described previously. Next, 450 mL of assay medium (resulting in 525 mL final volume) was added. Cells were incubated in a 37° C. incubator without CO2 for 1 hour to allow for pre-equilibration with the assay medium. Pre-warmed oligomycin, FCCP, rotenone, and antimycin A were loaded into injector ports A, B, and C of the sensor cartridge, respectively. The final concentrations of injections were as follows: for the cell number optimization experiment, 0.25 mM oligomycin, 1 mM FCCP, and 1 mM rotenone & antimycin A. The cartridge was calibrated using the XF24 analyzer (Seahorse Bioscience, Billerica, MA, USA), and the assay proceeded using the cell mito stress test assay protocol. Oxygen consumption rate (OCR) was measured under basal conditions, followed by the sequential addition of oligomycin, FCCP, and rotenone. This process allowed for the estimation of the contribution of individual parameters for basal respiration, proton leak, maximal respiration, non-mitochondrial respiration, and ATP production.

Glycolysis stress test assay: Cells were grown on BEASTS surfaces of varying stiffness levels (8 kPa, 25 kPa, and 55 kPa) for four days, followed by transfer to XFe24 culture plates. After a 24-hour incubation, the cells were exposed to injections of glucose, oligomycin, and 2-DG in assay medium. The assay was conducted using the glycolytic stress test assay protocol, and ECAR was measured under different conditions. A control group was included, receiving injections of the assay medium. The experiment allowed for the evaluation of the contribution of individual parameters, such as non-glycolytic acidification, glycolysis, glycolytic capacity, and glycolytic reserve of htr8 cells with various stiffness conditions.

ROS Assay: The intracellular production of reactive oxygen species (ROS) as a function of substrate stiffness was assessed using the 5-(and-6)-Chloromethyl-2′, 7′-dichlorodihydrofluorescein diacetate (CM-H2DCFDA) dye. This chemically reduced form of fluorescein serves as an indicator for ROS in cells. Upon oxidation, the non-fluorescent H2DCFDA is converted to the highly green fluorescent 2′,7′-dichlorofluorescein. After culturing cells on BEASTS substrates with varying stiffness levels (8 kPa, 25 kPa, and 55 kPa) for four days, the culture media was removed, and the cells were washed with warm phosphate-buffered saline (PBS). A 2 mM CM-H2DCFDA solution [Life Technologies] in PBS was added to each well and incubated at 37° C. for 30 minutes. Subsequently, the cells were washed three times with PBS to remove excess dye. Fluorescent images of the cells were obtained using a ZEISS Axioscope 5 fluorescent microscope, which allowed for the visualization of ROS production as a function of substrate stiffness in the cultured cells.

GSH Assay: The GSH assay was conducted using the GSH-Glo™ Glutathione Assay (Promega Corporation, USA) to evaluate intracellular glutathione (GSH) production in cells cultured on BEASTS substrates with varying stiffness levels (8 kPa, 25 kPa, and 55 kPa). First, 50,000 cells were seeded and allowed to attach in white, clear-bottom 96-well plates containing 100 μL of complete culture medium. Following cell attachment, the intracellular production of GSH was assessed using the GSH-Glo™ Glutathione Assay (Promega Corporation, USA), as per the manufacturer's instructions. In brief, after the incubation period, the culture medium was replaced with GSH-Glo Reagent and incubated for 30 minutes. Subsequently, the luciferin detection reagent was added to each well. The plate was then incubated for an additional 15 minutes at room temperature to allow for luminescence development. Glutathione detection and quantification were carried out in reference to the standard GSH curve, prepared according to the kit's guidelines. Luminescence readings of the plates were obtained using a plate reader, providing insight into the relationship between substrate stiffness and intracellular GSH production.

Immunostaining assay: After culturing cells on BEASTS substrates with varying stiffness levels (8 kPa, 25 kPa, and 55 kPa) for four days, cells were fixed using 4% paraformaldehyde in PBS at room temperature for 30 minutes, and permeabilized with 0.2% Triton X-100 for 15 minutes at room temperature. To block unspecific binding of the antibodies, cells were incubated with 1% BSA for 30 minutes. Cells were then incubated with the diluted primary antibody in 1% BSA overnight at 4° C. The solution was decanted, and the cells were washed three times in PBST. Subsequently, cells were incubated with the secondary antibody in 1% BSA for 1 hour at room temperature in the dark. After decanting the secondary antibody solution, cells were washed three times with PBS for 5 minutes each in the dark. DAPI was used to stain the cell nuclei by incubating the samples in a 1 mg/mL solution for 10 minutes at room temperature. Confocal microscopy was employed to acquire fluorescent images using appropriate filters to visualize the antibody staining and cell nuclei.

Substrate characterization: The polymers Poly(diallyldimethylammoniumchloride) (PDAC) in a 20 wt % solution, poly(sodium 4-styrenesulfonate) (SPS) with molecular weight 70,000, poly(D-glucosamine)* with molecular weight 25,000 were purchased from Sigma-Aldrich (St. Louis, MO). The polyelectrolyte multilayer films (PEM) consist of polydimethylsiloxane (PDMS) and polysodium 4-styrenesulfonate (SPS), a nanomaterial polymer used in layer-by-layer assembly. PDMS was chosen as prior studies have used this polymer type to achieve elastic moduli range spanning three orders while keeping other variables constant. PDMS polymers were chosen as these are widely implemented to represent soft tissue in vitro and mimic soft-tissue implants in vivo. Two commercially available polymers, Sylgard 184 (crosslinker) and Sylgard 527 are used in creating the stiffness gels of the BEASTS systems. The schematic and major findings of this study presented were plotted in Biorender.com.

Mechanical characterization of stiffness gels: The stiffness of the PDMS gels was characterized using a TX-XT2 Texture Analyzer (Texture Technologies Corp). Samples were measured by the amount of force with respect to the probe radius used and displacement with results plotted in GraphPad Prism 9.5. The modulus was measured using the following formula: F=8×G×d×h, where: F=Force (N), G=Modulus (Pa), d=radius of probe (mm), h=displacement (mm), as determined by indentation to characterize the poroelasticity of gels.

Data Sets Review: Analysis of differential gene expression in aged brain tissue compared with young, healthy tissues was performed using the publicly available dataset in the Gene Expression Omnibus (GEO) Series GSE33000. This dataset was selected due to its large size and comprehensive information on ages, type of brain disorders, and comprehensive gene identification and information. This dataset was accessed through the GEO2R interactive web tool (BioProject ID: 146519; Accession PRJNA146519). This dataset contains information on 624 patient samples obtained by the Harvard Brain Tissue Resource Center (HBTRC) submitted in 2011. The total RNA from each sample was processed in a custom-made Agilent 44K array (GPL4372).

TABLE 1
GSE33000 experimental groups chosen for this study.

| | Sample | Range | Mean age ± |
|---|---|---|---|
| Group | size (n) | (years) | SD (years) |
| Less than 40 years, (<40) | 24 | 18-39 | 32 ± 6.87 |
| 40 years and older, (≥40) | 290 | 40-106 | 62 ± 10.68 |

From this dataset, data was extracted on all tissue of 40 years and older (n=290) and less than 40 years (n=24) which had gene expression data available. The data was selected at the years of less than 40 and 40 and older for multiple reasons: (1) clinical relevance: the age of 40 is often used as a cutoff point in clinical trials and research, as many medical conditions become more prevalent or severe after this age 23-30. By splitting the sample into two groups based on this cutoff, one can better examine the clinical relevance of the genomic approach here for the different age groups; (2) Biological challenges: aging is associated with many biological changes such as cellular senescence, accumulation of DNA damage, and here, stiffening of cellular ECM appears to beas a driver of aging. These changes may be more pronounced in individuals over the age of 40. By splitting the groups, there was a better comparison of the biological changes that occur within each group; (3) Behavioral changes: as people age, they often experience changes in their lifestyle and behavior that may affect their health and well-being. The data was separated based on these lifestyles to account for the variance that might be caused; (4) Statistical power: the overall statistical power by dividing the two groups at these cutoff increases by reducing the variability of each group and detecting meaningful differences between the groups.

Bioinformatic approach: To reduce the dimensionality of the large data set, a Principal Component Analysis (PCA) of the transcriptome was performed in aging humans. To compute the PCA, GraphPad Prism 9.5 was used to input genes as the eigenvector and age as the group. Gene enrichment analysis was performed by the ToppGene Suite (https://toppgene.cchmc.org/prioritization.jsp). Selected Gene Ontology (GO) of Molecular Function, Cellular Component, and Biological Process were plotted. Disease enrichment was plotted from SRplot (http://www.bioinformatics.com.cn/srplot). A volcano plot was performed on the log fold change of differentially expressed genes (DEGs) with a log 10 p value and plotted using GraphPad Prism 9.5. The DEGs were further grouped based on their functionality and significance as a heatmap using GraphPad Prism 9.5.

BV2 mouse microglia and HMC3 human cells: All cells were grown in aseptic conditions following standard cell culture protocol and stored in an incubator set at 37° C. with 5% CO2. BV2 mouse microglia and HMC3 human were grown in Dulbecco's Modified Eagle Medium/Ham's F-12 Nutrient Mixture (DMEM/F12) and supplemented with 10% FBS, 1% PS, 1% Non-Essential Amino Acid (NEAA), and 1% Sodium Pyruvate. Additionally, recombinant mouse granulocyte-macrophage colony-stimulating factor (GM-CSF) from Shenandoah Biotechnology Inc-Protein Pros was added to the BV2 culture flask at a concentration of 10 ng/mL and replenished every 3 days. Once the microglia were seeded onto the BEASTS platform, GM-CSF was no longer added to the media to determine the impact of stiffness alone without changes to the media. Passages were kept at a low number between 15-25.

Cell Count and Growth Analysis: Microglia were seeded in triplicate (7.5×10⁵ cells/well) in a 12-well plate and the growth of cells was counted every other day, after which the stiffer matrices reached 100% confluency. At 48 h intervals, cells were trypsinized and counted using trypan blue exclusion tests. The doubling time of each stiffness was calculated with the following formula: C_end-C_start x 2(t/T), where C_start is the number of cells at the beginning (day 0), C_end is the number of cells after a period of time t and T is the doubling time.

Cell Viability Assay: The BV2 microglial cells (a gift from Paul Blum, University of Nebraska-Lincoln) were plated (7.5×10⁵ cells/well) in 12-well plates on 2 kPa, 8 kPa, 15 kPa, and 25 kPa stiffnesses for 24, 72, 120, 168, 216, and 264 h. After incubation time, 3-(4,5-dimethylthiazol-2-yl)-2,5-diphenyl tetrazolium bromide (MTT) was added to reach a final concentration of 0.5 mg/ml., and incubated for a further 4 h. The absorbance was detected at 570/620 nm using a Synergy™ 2 multi-mode microplate reader (BioTek, VT, USA).

Bioenergetic profile: Cells were grown on stiffness for 11 days. Cells were then plated at 80,000 cells/well in a Seahorse XF 24-Cell Culture Microplate (Agilent). All Seahorse experiments were performed at 24 hours after individual stimuli. At the end of treatment, cells were washed twice with Agilent Seahorse XF Media (Agilent). A final volume of 500 μL was placed in each well. Cells were then incubated in a 5% CO2 chamber at 37° C. for 1 hour before being placed into a Seahorse XFe24 Analyzer (Agilent). For OCR experiments, cells were treated with 1 μM oligomycin, 2 μM carbonyl cyanide p-trifluoromethoxy phenylhydrazone (FCCP), and 0.5 μM rotenone in Seahorse mitochondria stress test media. A total of three OCR measurements were taken after each compound was administered. For extracellular acidification rate (ECAR) experiments, cells were treated with 10 mM glucose, followed by 1 μM oligomycin, followed by 50 mM 2-deoxyglucose in Seahorse glycolysis stress test media. Three measurements were taken prior to the first injection and after each injection.

Scratch wound migration assay: The microglia cells (7.5×10⁵ cells/well) were plated in 12-well plates on 2 kPa, 8 kPa, 15 kPa, and 25 kPa stiffnesses until cells reached confluency. A scratch was gently performed in each of the wells with a sterile 200 μL pipette tip, followed by two washes of PBS to remove floating cells. Cells were then treated with complete media and pictures were taken every 15 minutes to monitor progress. Photos were taken of the same region of the scratch. Quantification of cell migration was done using ImageJ, by counting the total number of cells in the field and the number of cells present in the slit using an established and optimized protocol for identification and accuracy 35. Briefly, the ImageJ macro detects the edges of the scratch and quantifies the closure while counting the number of cells in the field. The result was then plotted in GraphPad Prism 9.5.

ROS Quantification and Imaging: 5-(and6)-Chloromethyl-2′,7′-dichlorodihydro-fluorescein diacetate (CM-H2DCFDA) is a fluorescent indicator activated by the presence of ROS. The intensity of CM-H2DCFDA was measured by calculating the corrected total cell fluorescence (CTCF) from The Open Lab Book Imaging protocols. Microglial cells were plated onto 12-well plates at a density of 75,000 cells/mL. Microglial cells were grown to confluency and then 10 μM DCF-DA (Invitrogen) was added to all wells, and cells were incubated for a further 30 minutes before analysis of ROS-activated DCF-DA fluorescence (FL-1/525 nm).

Cholesterol Detection Assay: Detection of cholesterol using a cholesterol dehydrogenase and esterase was performed using Cholesterol/Cholesterol Ester-Glo™ Assay (Cat #J3190; Promega, Madison WI, USA) according to the manufacturer's instructions. Briefly, microglial cells were grown in 12-well plates on differing stiffness for 11 days and then plated with or without esterase to remove fatty acid from esters to produce total cholesterol. Total luminescence was measured using a Synergy™ 2 multi-mode microplate reader (BioTek, VT, USA).

BODIPY 493/503 in vitro staining: Microglial cells were seeded at 1.5×10⁵ cells on 6-well BEASTS plates. Following 11 days on stiffness, cells were fixed in 4% paraformaldehyde (PFA) for 30 min, washed two times in PBS, and incubated in PBS with BODIPY 493/503 (1:1,000 from a 1 mg-1 stock solution in DMSO; Thermo Fisher) for 20 min at room temperature. Cells were washed twice in PBS and pictures were taken under Zeiss Axiovert 40 CFL Inverted microscope (Göttingen, Germany).

Actin staining and confocal microscopy: HMC3 microglia were seeded in a 12-well plate and cultured for 11 days on 2, 8, and 25 kPa. On day 11, HMC3 cells were fixed with 4% paraformaldehyde for 40 minutes. Cells were washed with 1×PBS and then stained with DAPI (1:1000 dilution). The actin solution was prepared with 2 drops per mL and incubated for 25 minutes. Cells were washed with 1×PBS and examined with a laser scanning confocal microscope (Nikon TE-300 Inverted Fluorescence Microscope). DAPI was measured under excitation wavelengths of 358 nm and Alexa Fluor 488 was used for Actin and FITC. Acquisition by this microscope was z-stacked, which is a compilation of images taken between the first and last planes of focus, for maximum focus. Total magnification was 30× objective. Actin and DAPI signals were normalized to 2 kPa to quantify fluorescence.

Lactate Dehydrogenase Activity Assay: Detection of lactate dehydrogenase (LDH) activity using a reductase was performed using LDH-Glo™ Assay (Cat #J2380; Promega, Madison WI, USA) according to the manufacturer's instructions. Briefly, microglial cells were grown in 12-well plates on differing stiffness for 11 days and then plated with reductase. The positive control group was treated with 0.1% Triton X-100 (Thermo Scientific, Waltham, MA, USA) and was considered 100%. Total luminescence was measured using a Synergy™ 2 multi-mode microplate reader (BioTek, VT, USA)

Total GSH/GSSH Detection Assay: Detection of GSH and oxidized glutathione (GSSG) using a GSH probe, Luciferin-NT, was performed using GSH/GSSG-Glo™ Assay (Cat #V6611; Promega, Madison WI, USA) according to the manufacturer's instructions. Briefly, microglial cells were grown in 12-well plates on differing stiffness for 11 days and then plated with or without a blocking agent for GSH, leaving GSSG intact. Total luminescence was measured using a Synergy™ 2 multi-mode microplate reader (BioTek, VT, USA).

Caspase-1 and Non-canonical Inflammasome Activity Assays: Detection of caspase-1 and non-canonical caspases using a luciferase assay was performed using Caspase-Glo™ Inflammasome Assay (Cat #G9951; Promega, Madison WI, USA) according to the manufacturer's instructions. Briefly, microglial cells were grown in 12-well plates on differing stiffness for 11 days and then plated with a selective caspase-1 substate, Z-WEHD, for catalytically active caspase-1 with or without a caspase-1 specific inhibitor, Ac-YVAD-CHO, to confirm specific activity in parallel samples. The Ac-YVAD-CHO has minimal effect on other cross-reacting caspases 3, 5, and 6, thereby, representing non-canonical inflammatory caspase activity. Total luminescence was measured using a Synergy™ 2 multi-mode microplate reader (BioTek, VT, USA).

Urea based assay: Urea secretion in microglial culture medium was quantified using Stanbio Urea Nitrogen (BUN) kit (Stanbio, Boerne, TX) using the manufacturer's instructions. Briefly, the kit utilizes the reaction between urea and diacetyl monoxime which results in a color change that can be quantified at an absorbance of 520 nm read on Synergy™ 2 multi-mode microplate reader (BioTek, VT, USA).

DNA Damage Competitive ELISA: DNA damage in microglial culture media samples was quantified using DNA Damage competitive ELISA kit from Life Technologies (Carlsbad, CA) according to the manufacturer's instructions. Briefly, DNA damage is detected by three oxidized guanine species: 8-hydroxy-2′-deoxyguanosine (8-OHdG) from DNA, RNA, and digested DNA from DNA or RNA which may be present in cell culture medium. A 96-well plate coated with antibodies had standards/samples added for 2 hours. The chromogen, tetramethylbenzidine (TMB) substrate solution was then added for 30 minutes. Stop solution was added and the plate was read at 450 nm within 10 minutes of adding the stop solution on Synergy™ 2 multi-mode microplate reader (BioTek, VT, USA). The limit of detection was 8,000 μg/mL 8-OHdG.

NAC administration in microglial cells: NAC was purchased (Sigma-Aldrich, St. Louis, MO, USA) and was used in treatment after seeding microglial cells for 3 hours with a 2×PBS wash, then replaced with microglia media every 72 hours. Concentrations of 0.25 and 2.5 mM were used 36. Microglia were grown for 11 days and then assessed using qRT-PCR, counting, and luminescent assays.

Gene Expression by Quantitative RT-PCR (qRT-PCR): Total RNA expression was extracted from microglial cells using TRIzol reagent (Invitrogen, CA, USA) and RNA samples were quantified by Take3 Micro-Volume Plate reader (BioTek, VT, USA) by spectrophotometry at 260/280 nm. Purity was assessed by an absence of 320 nm. RNA samples were reverse transcribed using the M-MuLV RT reagent Kit (New England BioLabs, MA, USA). Relative mRNA levels were determined by real-time PCR using a SYBR® PowerUp master mix and a real-time PCR detection system (Mastercycler Realplex ep gradient, Eppendorf, Hamburg, Germany). Data were expressed in Ct values normalized to 18S and f40 and older change between control (2 kPa) and treated groups (8, 15, 25 kPa) was determined using the 2-ΔΔCt method. The telomerase-associated genes were probed using the TaqMan™ Array Human Telomere Extension by Telomerase (Applied Biosystems #4414187) with multiple plates. Briefly, samples were loaded with SYBR® PowerUp master mix and a real-time PCR detection system (Mastercycler Realplex ep gradient, Eppendorf, Hamburg, Germany). Data were expressed in Ct values normalized to endogenous controls provided with the array and f40 and older change between groups was determined using the 2-ΔΔCt method.

Statistical Analyses for aging studies: Data are presented as the mean #S.E.M. Statistical analyses were performed using GraphPad Prism 9. The statistical significance was determined based on multiple t-tests multiple student's t-tests, correct for multiple comparisons using the Bonferroni-Dunn method, and p values<0.05 were considered statistically significant. For samples with different sample sizes, a Welch's t-test for unequal variances was used to account for possible type 1 error rates 37. The number of experiments is indicated in the Figure legends.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A cell culture system with tunable stiffness comprising a biocompatible polydimethyl siloxane substrate and a polyelectrolyte multilayer.

2. The cell culture system of claim 1, wherein the polyelectrolyte multilayer is present as a plurality of films providing a three dimensional cellular microenvironment within the substrate.

3. The cell culture system of claim 1, wherein the tunable stiffness is characterized by a Young's modulus ranging from about 2 kilopascal (kPa) to about 60 kPa.

4. The cell culture system of claim 1, wherein the substrate and the polyelectrolyte multilayer are combined without the addition of any adhesive cell component.

5. The cell culture system of claim 1, further comprising primary human hepatocytes.

6. The cell culture system of claim 5, further comprising one or more of hepatic stellate cells, cholangiocytes, and liver sinusoidal endothelial cells.

7. The cell culture system of claim 1, further comprising trophoblasts.

8. The cell culture system of claim 7, further comprising one or more of endothelial cells, epithelial cells, fibroblasts, and mesenchymal stromal cells.

9. A cell culture system with tunable stiffness comprising a biocompatible polydimethyl siloxane substrate with a polyelectrolyte multilayer configured to support hepatocytes and defining a liver disease research model.

10. The cell culture system of claim 7, wherein the liver disease is one or more of alcoholic liver disease, non-alcoholic fatty liver disease, chronic viral hepatitis, hepatocellular carcinoma, cirrhosis, primary biliary cirrhosis, and primary sclerosing cholangitis.

11. A cell culture system with tunable stiffness comprising a biocompatible polydimethyl siloxane substrate with a polyelectrolyte multilayer configured to support trophoblasts and defining a human placenta research model mimicking a pregnancy associated disorder.

12. The cell culture system of claim 11, wherein the pregnancy associated disorder is preeclampsia, intra-uterine growth restriction, gestational diabetes mellitus, or polycystic ovary syndrome.

13. A cell culture system with tunable stiffness comprising a biocompatible polydimethyl siloxane substrate with a polyelectrolyte multilayer configured to support glial cells and defining a human brain model.

14. The cell culture system of claim 13, wherein the human brain model mimics aging or a neurological disorder.

15. The cell culture system of claim 14, wherein the neurological disorder is Alzheimer's disease, Parkinson's disease, or traumatic brain injury.
